(19) United States
(12) Patent Application Publication
(10) Pub. No.: US 2007/0299072 A1
(43) Pub. Date: Dec. 27, 2007

(54) KV1.5 POTASSIUM CHANNEL INHIBITORS

(75) Inventors: John Michael Janusz, West Chester, OH (US); Shengde Wu, West Chester, OH (US); Neil T. Fairweather, Liberty Township, OH (US); Wenlin Lee, San Diego, CA (US); Benjamin E. Blass, Eagleville, PA (US); Andrew J. Fluxe, Lebanon, OH (US); Stephen J. Hodson, Mason, OH (US); James M. Ridgeway II, Batavia, OH (US)

Correspondence Address: WOODCOCK WASHBURN LLP/WYETH, CIRA CENTRE, 12TH FLOOR, 2929 ARCH STREET, PHILADELPHIA, PA, 19104-2891, US

(73) Assignee: Wyeth, Madison, NJ (US)

(21) Appl. No.: 11/765,357

(22) Filed: Jun. 19, 2007

Related U.S. Application Data
(60) Provisional application No. 60/815,066, filed on Jun. 20, 2006.

(51) Int. Cl. A61K 31/438; A61K 31/496; A61K 31/5377; A61P 25/04; C07D 241/04; C07D 265/30; C07D 471/10

(52) U.S. Cl. 514/235.8; 514/253.09; 514/278; 544/230; 544/70; 546/20

(57) ABSTRACT

The present invention relates to 4-oxo-1,3,8-triaza-spiro[4.5]decanes which are useful as Kv1.5 potassium channel inhibitors providing atrial-selective antiarrhythmic activity. The present invention further relates to compositions and methods for treating atrial-selective antiarrhythmia.

### CROSS-REFERENCE TO RELATED APPLICATIONS

This application claims the benefit of U.S. Provisional Application No. 60/815,066 filed Jun. 20, 2006, which is herein incorporated by reference in its entirety.

### FIELD

The present invention relates, inter alia, to compounds effective as Kv1.5 potassium channel inhibitors. The present invention further relates to inter alia, compositions comprising said Kv1.5 potassium channel inhibitors, and to methods for treating cardiac arrhythmia.

### BACKGROUND OF THE INVENTION

Atrial fibrillation (AF) is the most frequently encountered cardiac arrhythmia in the clinical setting. It affects nearly 3 million people in the United States and its prevalence increases with the aging of the population. AF is most often treated with class III antiarrhythmic agents, acting at both the atrial and ventricular levels. Commonly used or prescribed antiarrhythmic drugs inhibit various potassium channels, and prolong ventricular repolarization. This prolongation can in turn precipitate the occurrence of life-threatening-ventricular arrhythmias, mainly Torsades de Pointes (TdP).

Atrial-selective antiarrhythmic agents offer the possibility of increased therapeutic efficacy and safety by minimizing cardiac proarrhythmia inherent in traditional antiarrhythmic therapies.

There is therefore a long felt need for atrial-selective antiarrhythmic agents which do not affect ventricular rhythm. In addition, there is a long felt need for atrial-selective antiarrhythmic agents which are compatible with other cardiac devices, protocols, therapies, and medications. The present invention addresses this and other needs.

### SUMMARY OF THE INVENTION

The 1-N-amino-2-imidazolidinones of the present invention are a new class of compounds. Compounds of this class have been found to inhibit Kv1.5 potassium channels function. The compounds of the present invention have formula I: 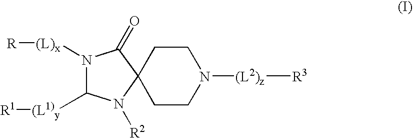
or a pharmaceutically acceptable salt thereof,
wherein R is optionally substituted phenyl;
R¹ is optionally substituted phenyl;
R² is hydrogen, optionally substituted C₁-C₆ linear or branched alkyl, optionally substituted C₃-C₆ cycloalkyl, or —C(O)R²³ wherein R²³ is optionally substituted C₁-C₆ linear or branched alkyl or optionally substituted C₃-C₆ cycloalkyl;
R³ is selected from:
i) hydrogen;
ii) optionally substituted C₁-C₆ linear or branched alkyl or optionally substituted C₃-C₆ cycloalkyl;
iii) —C(O)R⁴;
wherein R⁴ is optionally substituted C₁-C₆ linear or branched alkyl, optionally substituted C₃-C₆ cycloalkyl, optionally substituted heterocycle, optionally substituted aryl, or optionally substituted heteroaryl;
   iv) —C(O)NR⁵R⁶;
wherein R⁵ and R⁶ are each independently selected from
a) hydrogen;
b) optionally substituted C₁-C₆ linear or branched alkyl;
c) optionally substituted C₃-C₇ cycloalkyl;
d) —OR⁷;

wherein R⁷ is hydrogen or optionally substituted C₁-C₆ linear or branched alkyl;
e) —NR⁸R⁹;
   wherein R⁸ and R⁹ are each independently hydrogen, optionally substituted C₁-C₆ linear or branched alkyl, optionally substituted C₁-C₆ linear or branched alkoxy, —OH, or —CO₂R¹⁰, wherein R¹⁰ is optionally substituted C₁-C₆ linear or branched alkyl; or R⁸ and R⁹ can be taken together with the atom to which they are bound to form an optionally substituted ring having from 3 to 7 ring atoms and optionally containing one or more additional heteroatom ring atoms independently selected from N, O, or S; or
   f) R⁵ and R⁶ can be taken together with the atom to which they are bound to form an optionally substituted ring having from 3 to 7 ring atoms and optionally containing one or more additional heteroatom ring atoms independently selected from N, O, or S;
      v) —C(NR¹¹)R¹²;
wherein R¹¹ is
a) hydrogen;
b) optionally substituted C₁-C₆ linear or branched alkyl or optionally substituted C₃-C₆ cycloalkyl;
c) —OH; or
d) —CN;
and R¹² is
a) optionally substituted C₁-C₆ linear or branched alkyl or optionally substituted C₃-C₆ cycloalkyl;
b) —OR¹³;
   R¹³ is hydrogen, optionally substituted C₁-C₆ linear or branched alkyl, optionally substituted C₃-C₆ cycloalkyl, or optionally substituted aryl; or
c)—NR¹⁴R¹⁵;
   R¹⁴ and R¹⁵ are each independently hydrogen, optionally substituted aryl, optionally substituted C₁-C₆ linear or branched alkyl, or optionally substituted C₃-C₈ cycloalkyl;
      vi) —SO₂R¹⁶;
wherein R¹⁶ is optionally substituted aryl, optionally substituted C₁-C₆ linear or branched alkyl, or optionally substituted C₃-C₆ cycloalkyl; or
   vii) —C(O)R¹⁷;
wherein R¹⁷ is optionally substituted aryl or optionally substituted heteroaryl;
   viii) —C(O)OR¹⁸;
wherein R¹⁸ is optionally substituted C₁-C₆ linear or branched alkyl, optionally substituted C₃-C₆ cycloalkyl, or optionally substituted aryl;
   L, L¹, and L² are each independently:
   —[C(R¹⁹)₂]ₙ—
   each R¹⁹ is, at each occurrence, independently selected from hydrogen, methyl, or ethyl;
   n is 1 to 4; and
   x, y, and z are each independently 0 or 1.

Compounds of the present invention include those in which:

R is optionally substituted phenyl;

R¹ is optionally substituted phenyl;

R² is hydrogen, C₁-C₆ linear or branched alkyl, C₃-C₆ cycloalkyl, or —C(O)R²³ wherein

R²³ is C₁-C₆ linear or branched alkyl or C₃-C₆ cycloalkyl;

R³ is selected from:

i) hydrogen;

ii) C₁-C₆ linear or branched alkyl or C₃-C₆ cycloalkyl;

iii) —C(O)R⁴;

wherein R⁴ is optionally substituted C₁-C₆ linear or branched alkyl, optionally substituted C₃-C₆ cycloalkyl, optionally substituted heterocycle, optionally substituted aryl, or optionally substituted heteroaryl;
   iv) —C(O)NR⁵R⁶;
wherein R⁵ and R⁶ are each independently selected from
a) hydrogen;
b) C₁-C₆ linear or branched alkyl;
c) C₃-C₇ cycloalkyl;
d) —OR⁷;
   wherein R⁷ is hydrogen or C₁-C₆ linear or branched alkyl;
e) —NR⁸R⁹;
   wherein R⁸ and R⁹ are each independently hydrogen, C₁-C₆ linear or branched alkyl, C₁-C₆ linear or branched alkoxy, —OH, or —CO₂R¹⁰ wherein R¹⁰ is C₁-C₆ linear or branched alkyl; or R⁸ and R⁹ can be taken together with the atom to which they are bound to form an optionally substituted ring having from 3 to 7 ring atoms and optionally containing one or more additional heteroatom ring atoms independently selected from N, O, or S; or
f) R⁵ and R⁶ can be taken together with the atom to which they are bound to form an optionally substituted ring having from 3 to 7 ring atoms and optionally containing one or more additional heteroatom ring atoms independently selected from N, O, or S;
   v) —C(NR¹¹)R¹²;
wherein R¹¹ is
a) hydrogen;
b) C₁-C₆ linear or branched alkyl or C₃-C₆ cycloalkyl;
c) —OH; or
d) —CN;
and R¹² is
a) C₁-C₆ linear or branched alkyl or C₃-C₆ cycloalkyl;
b) —OR¹³;
   wherein R¹³ is hydrogen, C₁-C₆ linear or branched alkyl, C₃-C₆ cycloalkyl, or phenyl; or
c)—NR¹⁴R¹⁵;
   R¹⁴ and R¹⁵ are each independently hydrogen, phenyl, C₁-C₆ linear or branched alkyl, or C₃-C₆ cycloalkyl;
      vi) —SO₂R¹⁶;
wherein R¹⁶ is phenyl, C₁-C₆ linear or branched alkyl or C₃-C₆ cycloalkyl;
   vii) —C(O)R¹⁷;
wherein R¹⁷ is aryl or C₁-C₅ heteroaryl;
   viii) —C(O)OR¹⁸;
wherein R¹⁸ is C₁-C₆ linear or branched alkyl, C₃-C₆ cycloalkyl, or phenyl;
   L, L¹, and L² are each independently:
   —[C(R¹⁹)₂]ₙ—
   each R¹⁹ is, at each occurrence, independently selected from hydrogen, methyl, or ethyl;
   n is 1 to 4; and
   x, y, and z are each independently 0 or 1; or a pharmaceutically acceptable salt form thereof.

Compounds of the present invention include those in which:

R is optionally substituted phenyl;

R¹ is optionally substituted phenyl;

R² is hydrogen, C₁-C₄ linear or branched alkyl, or C₃-C₄ cycloalkyl;

R³ is selected from:

i) hydrogen;

ii) C₁-C₆ linear or branched alkyl or C₃-C₆ cycloalkyl;

iii) —C(O)R⁴;

wherein R⁴ is C₁-C₆ linear or branched alkyl or C₃-C₆ cycloalkyl;
   iv) —C(O)NR⁵R⁶;
wherein R⁵ and R⁶ are each independently selected from
a) hydrogen;
b) C₁-C₆ linear or branched alkyl;
c) C₃-C₇ cyclic alkyl;
d) —OR⁷;
   wherein R⁷ is hydrogen or C₁-C₆ linear or branched alkyl;
e) —NR⁸R⁹;
   wherein R⁸ and R⁹ are each independently hydrogen, C₁-C₆ linear or branched alkyl, C₁-C₆ linear or branched alkoxy, —OH, —CO₂R¹⁰ wherein R¹⁰ is C₁-C₆ linear or branched alkyl; or R⁸ and R⁹ can be taken together to form a ring having from 3 to 7 ring atoms; or
f) R⁵ and R⁶ can be taken together to form a ring having from 3 to 7 ring atoms;
   v) —C(NR¹¹)R¹²;
wherein R¹¹ is
a) hydrogen;
b) C₁-C₆ linear or branched alkyl;
c) —OH; or
d) —CN;
and R¹² is
a) C₁-C₆ linear or branched alkyl;
b) —OR¹³;
   R¹³ is hydrogen, C₁-C₆ linear or branched alkyl, or phenyl; or
c) —NR¹⁴R¹⁵;
   R¹⁴ and R¹⁵ are each independently hydrogen, or C₁-C₆ linear or branched alkyl;
      vi) —SO₂R¹⁶;
wherein R¹⁶ is phenyl; or C₁-C₆ linear or branched alkyl;
   vii) —C(O)R¹⁷;
wherein R¹⁷ is C₁-C₅ heteroaryl;
   viii) —C(O)OR¹⁸;
wherein R¹⁸ is C₁-C₆ linear or branched alkyl;
   L, L¹, and L² are each independently:
   —[C(R¹⁹)₂]ₙ—
   each R¹⁹ is, at each occurrence, independently chosen from hydrogen, methyl, or ethyl;

n is 1 to 4; and

x, y, and z are each independently 0 or 1; or a pharmaceutically acceptable salt form thereof.

The present invention further relates to compositions comprising: an effective amount of one or more compounds according to the present invention and an excipient.

The present invention also relates to a method for treating or preventing cardiac arrhythmias, including, for example, atrial fibrillation and atrial flutter, said method comprising administering to a subject an effective amount of a compound or composition according to the present invention.

The present invention yet further relates to a method for treating or preventing cardiac arrhythmias, including, for example, atrial fibrillation and atrial flutter, wherein said method comprises administering to a subject a composition comprising an effective amount of one or more compounds according to the present invention and an excipient.

The present invention also relates to a method for treating or preventing disease or conditions associated with cardiac arrhythmias, including, for example, thromboembolism, stroke, and heart failure. Said methods comprise administering to a subject an effective amount of a compound or composition according to the present invention.

The present invention yet further relates to a method for treating or preventing disease or conditions associated with cardiac arrhythmias, including, for example, thromboembolism, stroke, and heart failure, wherein said method comprises administering to a subject a composition comprising an effective amount of one or more compounds according to the present invention and an excipient.

The present invention further relates to a process for preparing the Kv1.5 potassium channel inhibitors of the present invention.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

### DETAILED DESCRIPTION OF THE INVENTION

The Kv1.5 potassium channel inhibitors of the present invention are capable of treating and preventing arrhythmia in the atrial portion of the human heart or in the heart of certain animals. It has been discovered that functional Kv1.5 potassium channels are found in human atrial tissue but not in human ventricular myocytes. Without wishing to be limited by theory, it is believed the inhibition of the Kv1.5 voltage-gated Shaker-like potassium (K⁺) ion channel can ameliorate, abate, or otherwise cause to be controlled, atrial fibrillation and flutter without prolonging ventricular repolarization.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

As used herein, unless otherwise noted, “alkyl” whether used alone or as part of a substituent group refers to straight and branched carbon chains having 1 to 20 carbon atoms or any number within this range, for example, 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂-amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Alkenyl and alkynyl groups can be optionally substituted. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “aryl,” wherein used alone or as part of another group, is defined herein as an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The terms “heterocyclic” and/or “heterocycle,” whether used alone or as part of another group, are defined herein as one or more rings (e.g., 2 or 3 rings) having from 3 to 20 atoms wherein at least one atom in at least one ring is a heteroatom selected from nitrogen (N), oxygen (O), or sulfur (S) and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom selected from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R⁸ and R⁹ taken together with the N to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

The terms “treat” and “treating,” as used herein, refer to partially or completely alleviating, inhibiting, ameliorating and/or relieving a condition from which a patient is suspected to suffer.

As used herein, “therapeutically effective” refers to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

For the purposes of the present invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula: 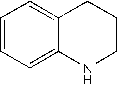
is, for the purposes of the present invention, considered a heterocyclic unit. 6,7-Dihydro-5H-cyclopentapyrimidine having the formula: 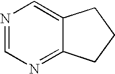
is, for the purposes of the present invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula: 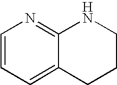
is, for the purposes of the present invention, considered a heteroaryl unit.

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁, alkyl; trifluoromethyl is a substituted C₁, alkyl; 4-hydroxyphenyl is a substituted aromatic ring; (N,N-dimethyl-5-amino)octanyl is a substituted C8 alkyl; 3-guanidinopropyl is a substituted C₃ alkyl; and 2-carboxypyridinyl is a substituted heteroaryl.

The variable groups defined herein, e.g., alkyl, alkenyl, cycloalkyl, heterocycle, aryl, and heteroaryl groups defined herein, whether used alone or as part of another group, can be optionally substituted with one or more substituents. Optionally substituted groups will be so indicated.

The following are non-limiting examples of substituents which can substitute for hydrogen atoms on a moiety: halogen (F, Cl, Br, I), —CN, —NO₂, oxo (═O), —OR²⁵, —SR²⁵, —N(R²⁵)₂, —NR²⁵C(O)R²⁵, —SO₂R²⁵, —SO₂OR²⁵, —SO₂N(R²⁵)₂, —C(O)R²⁵, —C(O)OR²⁵, —C(O)N(R²⁵)₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, alkenyl, alkynyl, haloalkyl, alkoxy, cycloalkyl, aryl, heterocycle, and heteroaryl groups is optionally substituted with 1-10 (e.g., 1-6 or 1-4) groups selected from halogen, —CN, —NO₂, oxo, and R²⁵; wherein R²⁵, at each occurrence, independently is hydrogen, —OR²⁶, —SR²⁶, —C(O)R²⁶, —C(O)OR²⁶, —C(O)N(R²⁶)₂, —SO₂R²⁶, —S(O)₂OR²⁶, —N(R²⁶)₂, —NR²⁶C(O)R²⁶, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R²⁵ units taken together with the atom(s) to which they are bound form an optionally substituted carbocycle or heterocycle wherein said carbocycle or heterocycle has 3 to 7 ring atoms; wherein R²⁶, at each occurrence, independently is hydrogen, C₁₋₆ alkyl, C₁₋₆ haloalkyl, C₂₋₈ alkenyl group, C₂₋₈ alkynyl group, cycloalkyl (e.g., C₃₋₆ cycloalkyl), aryl, heterocycle, or heteroaryl, or two R²⁶ units taken together with the atom(s) to which they are bound form an optionally substituted carbocycle or heterocycle wherein said carbocycle or heterocycle preferably have 3 to 7 ring atoms.

In some embodiments, the substituents are selected from

i) —OR²⁵; for example, —OH, —OCH₃, —OCH₂CH₃, —OCH₂CH₂CH₃;

ii) —C(O)R²⁵; for example, —COCH₃, —COCH₂CH₃, —COCH₂CH₂CH₃;

iii) —C(O)OR²⁵; for example, —CO₂CH₃, —CO₂CH₂CH₃, —CO₂CH₂CH₂CH₃;

iv) —C(O)N(R²⁵)₂; for example, —CONH₂, —CONHCH₃, —CON(CH₃)₂;

v) —N(R²⁵)₂; for example, —NH₂, —NHCH₃, —N(CH₃)₂, —NH(CH₂CH₃);

vi) —NR²⁵COR²⁵; for example, —NHCOCH₃, —NHCOCH₂CH₃, —NHCOC₆H₅;

vii) halogen; —F, —Cl, —Br, and —I;

viii) C₁-C₄ linear or branched haloalkyl; for example, —CH₂F, —CF₃, —CCl₃;

ix) —SO₂R²⁵; for example, —SO₂CH₃, —SO₂CH₂CH₃, —SO₂C₆H₅;

x) —SO₂N(R²⁵)₂; for example, —SO₂NH₂; —SO₂NHCH₃; —SO₂NHC₆H₅;

xi) C₁-C₆ linear or branched alkyl or C₃-C₆ cycloalkyl;

xii) cyano; and

xiii) nitro

wherein each R²⁵ is independently hydrogen, optionally substituted C₁-C₆ linear or branched alkyl (e.g., optionally substituted C₁-C₄ linear or branched alkyl) or optionally substituted C₃-C₆ cycloalkyl (e.g., optionally substituted C₃-C₄ cycloalkyl); or two R²⁵ units can be taken together to form a ring comprising 3 to 7 ring atoms. In certain aspects, each R²⁵ is independently hydrogen, C₁-C₆ linear or branched alkyl optionally substituted with halogen or C₃-C₆ cycloalkyl or C₃-C₆ cycloalkyl

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

For the purposes of the present invention the terms “compound,” “analog,” and “composition of matter” stand equally well for the Kv1.5 potassium channel inhibitors described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine

(e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R²⁰)₂, each R²⁰ may be the same or different than the other). Combinations of substituents and/or variables are permissible only if such combinations result in stable compounds.

Kv1.5 Potassium Channel Inhibitors

The Kv1.5 potassium channel inhibitors of the present invention are 5-spirocyclic-4-imidazolidinones, and include all enantiomeric and diasteriomeric forms and salts of compounds which are members of the genus named and referred to herein as 1-(R²-substituted)-2,3,8-(substituted)-4-oxo-1,3,8-triaza-spiro[4.5]decanes having the formula (I): 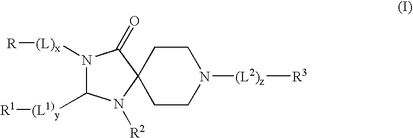
wherein the core scaffold is numbered in the following manner;

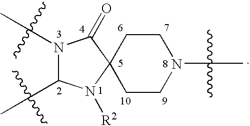

For the purposes of demonstrating the manner in which the compounds of the present invention are named and referred to herein, the compound having the formula: 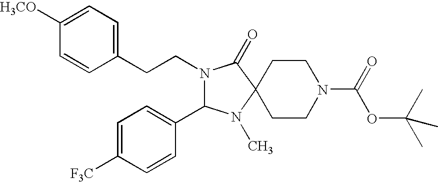
has the chemical name 1-methyl-2-(4-trifluoromethylphenyl)-3-[2-(4-methoxyphenyl)-ethyl]-4-oxo-1,3,8-triaza-spiro[4.5]decane-8-carboxylic acid tert-butyl ester.

For the purposes of the present invention, a compound depicted by the racemic formula, for example: 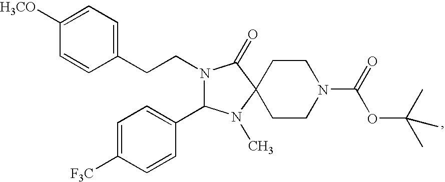
will stand equally well for either of the two enantiomers having the formula: 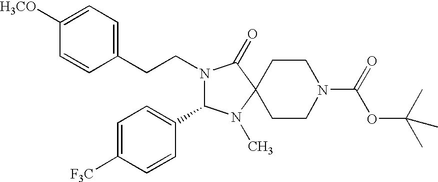
or the formula: 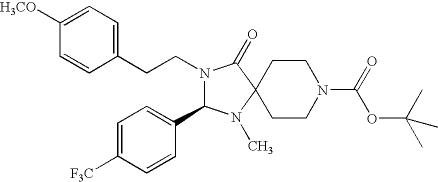
or mixtures thereof, or in the case where a second chiral center is present, all diastereomers. However, the term 5-spirocyclic-4-imidazolidinones is used in general to refer to the genus, which encompasses the compounds of the present invention, throughout the specification.

The particular embodiments and illustrations herein relating to particular aspects of the present invention may be combined in the compounds of the present invention.

In the present invention, R is optionally substituted phenyl. The phenyl group can be substituted with any of the substituents provided herein. Examples of suitable substituents include, but are not limited to halogen, optionally substituted C₁-C₆ linear or branched alkyl, optionally substituted C₁-C₆ linear or branched haloalkyl, optionally substituted C₃-C₆ cycloalkyl, —OR²⁰, —CN, —N(R²⁰)₂, —CO₂R²⁰, —C(O)N(R²⁰)₂, —NR²⁰C(O)R²⁰, —NO₂, and —SO₂R²⁰; each R²⁰ is independently hydrogen, optionally substituted C₁-C₆ (e.g., C₁-C₄) linear or branched alkyl, optionally substituted C₁-C₆ linear or branched haloalkyl, optionally substituted C₃-C₆ cycloalkyl (e.g., C₃-C₄ cycloalkyl), optionally substituted aryl, optionally substituted heterocycle, or optionally substituted heteroaryl; or two R²⁰ units can be taken together to form a ring comprising from 3 to 7 ring atoms. When two R²⁰ units are taken together to form a ring, the ring may comprise additional heteroatoms selected independently from oxygen, nitrogen, or sulfur; and the ring optionally may be substituted. Non-limiting examples of rings formed when two R²⁰ units are taken together include: piperidinyl, piperazinyl, morpholinyl, and pyrrolidinyl. In certain aspects, the substituents on the optionally substituted linear or branched alkyl group is a C₃-C₆ cycloalkyl. The phenyl group can be substituted at any position on the ring, e.g., meta, para, and/or ortho positions.

Exemplary embodiments of the present invention include a compound of Formula (I) or a pharmaceutically acceptable salt form thereof wherein R is phenyl, 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 3,4,5-trifluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 2,3,4-trichlorophenyl, 2,3,5-trichlorophenyl, 2,3,6-trichlorophenyl, 2,4,5-trichlorophenyl, 2,4,6-trichlorophenyl, or 3,4,5-trichlorophenyl.

Exemplary embodiments of the present invention include a compound of Formula (I) or a pharmaceutically acceptable salt form thereof wherein R is 2-methylphenyl, 3-methylphenyl, 4-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 3,4,5-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 3,5-diethylphenyl, 2,3,4-triethylphenyl, 2,3,5-triethylphenyl, 2,3,6-triethylphenyl, 2,4,5-triethylphenyl, 2,4,6-triethylphenyl, or 3,4,5-triethylphenyl.

Exemplary embodiments of the present invention include a compound of Formula (I) or a pharmaceutically acceptable salt form thereof wherein R is 2-cyclopropylphenyl, 3-cyclopropylphenyl, 4-cyclopropyl-phenyl, 2-(cyclopropylmethyl)phenyl, 3-(cyclopropylmethyl)phenyl, 4-(cyclopropyl-methyl)phenyl, 2-iso-butylphenyl, 3-iso-butylphenyl, 4-iso-butylphenyl, 2-tert-butylphenyl, 3-tert-butylphenyl, 4-tert-butylphenyl, 2-cyclobutylphenyl, 3-cyclobutyl-phenyl, 4-cyclobutylphenyl, 2-(cyclobutylmethyl)phenyl, 3-(cyclobutylmethyl)phenyl, or 4-(cyclobutyl-methyl)phenyl.

Exemplary embodiments of the present invention include a compound of Formula (I) or a pharmaceutically acceptable salt form thereof wherein R is 2-methoxyphenyl, 3-methoxyphenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 3,5-dimethoxyphenyl, 2,3,4-trimethoxyphenyl, 2,3,5-trimethoxyphenyl, 2,3,6-trimethoxy-phenyl, 2,4,5-trimethoxyphenyl, 2,4,6-trimethoxyphenyl, 2-hydroxyphenyl, 3-hydroxy-phenyl, 4-hydroxyphenyl, 2,3-dihydroxyphenyl, 2,4-dihydroxyphenyl, 2,5-dihydroxyphenyl, 2,6-dihydroxyphenyl, 3,4-dihydroxyphenyl, 3,5-dihydroxyphenyl, 2,3,4-trihydroxyphenyl, 2,3,5-trihydroxy-phenyl, 2,3,6-trihydroxyphenyl, 2,4,5-trihydroxyphenyl, or 2,4,6-trihydroxy-phenyl.

Exemplary embodiments of the present invention include a compound of Formula (I) or a pharmaceutically acceptable salt form thereof wherein R is 2-fluoromethoxyphenyl, 2-difluoromethoxyphenyl, 2-trifluoromethoxyphenyl, 3-fluoromethoxyphenyl, 3-difluoromethoxyphenyl, 3-trifluoromethoxyphenyl, 4-fluoromethoxyphenyl, 4-difluoromethoxyphenyl, 4-trifluoromethoxyphenyl, 2,4-bis(fluoromethoxy)phenyl, 2,4-bis(difluoromethoxy)phenyl, 2,4-bis(trifluoromethoxy)phenyl, 3,5-bis(fluoromethoxy)-phenyl, 3,5-bis(difluoromethoxy)phenyl, or 3,5-bis(trifluoromethoxy)phenyl.

Exemplary embodiments of the present invention include a compound of Formula (I) or a pharmaceutically acceptable salt form thereof wherein R is 2-cyanophenyl, 3-cyanophenyl, 4-cyanophenyl, 2,3-dicyano-phenyl, 2,4-dicyanophenyl, 2,5-dicyanophenyl, 2,6-dicyanophenyl, 3,4-dicyanophenyl, 2,3,4-tricyanophenyl, 2,3,5-tricyanophenyl, 2,3,6-tricyanophenyl, 2,4,5-tricyanophenyl, 3,4,5-tricyanophenyl, or 2,4,6-tricyanophenyl.

Exemplary embodiments of the present invention include a compound of Formula (I) or a pharmaceutically acceptable salt form thereof wherein R is 2-nitrophenyl, 3-nitrophenyl, 4-nitrophenyl, 2,3-dinitrophenyl, 2,4-dinitrophenyl, 2,5-dinitrophenyl, 2,6-dinitrophenyl, 3,4-dinitrophenyl, 3,5-dinitrophenyl, 2,3,4-trinitrophenyl, 2,3,5-trinitrophenyl, 2,3,6-trinitrophenyl, 2,4,5-trinitrophenyl, 3,4,5-trinitrophenyl, or 2,4,6-trinitropheny.

Exemplary embodiments of the present invention include a compound of Formula (I) or a pharmaceutically acceptable salt form thereof wherein R is 2,6-dimethyl-4-fluorophenyl, 2,6-dimethyl-3-fluorophenyl, 2,6-dimethyl-4-chlorophenyl, 2,6-di-tert-butyl-4-hydroxyphenyl, 2,6-difluoro-4-chlorophenyl, 2,6-difluoro-3-chlorophenyl, 2-hydroxy-4-methylphenyl, 2-hydroxy-5-methylphenyl, 2,6-dihydroxy-4-tert-butylphenyl, or 2,6-difluoro-4-cyanophenyl.

Exemplary embodiments of the present invention include a compound of Formula (I) or a pharmaceutically acceptable salt form thereof wherein R is 3-dimethylaminophenyl, 4-dimethylaminophenyl, 3-diethylaminophenyl, 4-diethylaminophenyl, 3-methylsulfanylphenyl, 4-methylsulfanyl-phenyl, 3-ethylsulfanylphenyl, 4-ethylsulfanylphenyl, 3-propylsulfanylphenyl, or 4-propylsulfanylphenyl.

Exemplary embodiments of the present invention include a compound of Formula (I) or a pharmaceutically acceptable salt form thereof wherein R is 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, or 4-(N,N-diethylamino)phenyl.

R¹ is optionally substituted phenyl. The phenyl group can be substituted with any of the substituents provided herein. Examples of suitable substituents include, but are not limited to: halogen, optionally substituted C₁-C₆ (e.g., C₁-C₄) linear or branched alkyl, optionally substituted C₁-C₆ linear or branched haloalkyl optionally substituted C₃-C₆ (e.g., C₃-C₄) cycloalkyl, —OR²¹, —CN, —N(R²¹)₂, —CO₂R²¹—C(O)N(R²¹)₂, —NR²¹C(O)R²¹, —SO₂R²¹, and —NO₂; each R²¹ is independently hydrogen, optionally substituted C₁-C₆ linear or branched alkyl (e.g., C₁-C₄ linear or branched alkyl), optionally substituted C₁-C₆ linear or branched haloalkyl, optionally substituted C₃-C₆ cycloalkyl (e.g., C₃-C₄ cycloalkyl), optionally substituted aryl, optionally substituted heteroaryl, or optionally substituted heterocycle; or two R²¹ units can be taken together to form a ring comprising from 3-7 ring atoms. When two R²¹ units are taken together to form a ring, the ring may comprise additional heteroatoms chosen from oxygen, nitrogen, or sulfur, and the ring optionally may be substituted. Non-limiting examples of rings formed when two R²¹ units are taken together include: piperidinyl, piperazinyl, morpholinyl, and pyrrolidinyl. In certain aspects, the substituent on the optionally substituted linear or branched alkyl group is a C₃-C₆ cycloalkyl. The phenyl group can be substituted at any position on the ring, e.g., meta, para, and/or ortho positions.

Exemplary embodiments of the present invention include a compound of Formula (I) or a pharmaceutically acceptable salt form thereof wherein R¹ is phenyl, 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 3,4,5-trifluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 2,3,4-trichlorophenyl, 2,3,5-trichlorophenyl, 2,3,6-trichlorophenyl, 2,4,5-trichlorophenyl, 2,4,6-trichlorophenyl, or 3,4,5-trichlorophenyl.

Exemplary embodiments of the present invention include a compound of Formula (I) or a pharmaceutically acceptable salt form thereof wherein R¹ is 2-methylphenyl, 3-methylphenyl, 4-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 3,4,5-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 3,5-diethylphenyl, 2,3,4-triethylphenyl, 2,3,5-triethylphenyl, 2,3,6-triethylphenyl, 2,4,5-triethylphenyl, 2,4,6-triethylphenyl, 3,4,5-triethylphenyl 2-isopropylphenyl, 3-isopropylphenyl, or 4-isopropylphenyl.

Exemplary embodiments of the present invention include a compound of Formula (I) or a pharmaceutically acceptable salt form thereof wherein R¹ is 2-cyclopropylphenyl, 3-cyclopropylphenyl, 4-cyclopropyl-phenyl, 2-(cyclopropylmethyl)phenyl, 3-(cyclopropylmethyl)phenyl, 4-(cyclopropyl-methyl)phenyl, 2-iso-butylphenyl, 3-iso-butylphenyl, 4-iso-butylphenyl, 2-tert-butylphenyl, 3-tert-butylphenyl, 4-tert-butylphenyl, 2-cyclobutylphenyl, 3-cyclobutyl-phenyl, 4-cyclobutylphenyl, 2-(cyclobutylmethyl)phenyl, 3-(cyclobutylmethyl)phenyl, or 4-(cyclobutylmethyl)phenyl.

Exemplary embodiments of the present invention include a compound of Formula (I) or a pharmaceutically acceptable salt form thereof wherein R¹ is 2-methoxyphenyl, 3-methoxyphenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 2,3,4-trimethoxyphenyl, 2,3,5-trimethoxyphenyl, 2,3,6-trimethoxyphenyl, 2,4,5-trimethoxyphenyl, 2,4,6-trimethoxyphenyl, 2-trifluoromethoxyphenyl, 3-trifluoromethoxyphenyl, 4-trifluoromethoxyphenyl, 2-trifluoromethyl-phenyl, 3-tri-fluoromethylphenyl, 4-trifluoromethylphenyl, 2-fluoromethoxyphenyl, 2-difluoromethoxyphenyl, 2-trifluoromethoxyphenyl, 3-fluoromethoxyphenyl, 3-difluoromethoxyphenyl, 3-trifluoromethoxyphenyl, 4-fluoromethoxyphenyl, 4-difluoromethoxyphenyl, 4-trifluoromethoxyphenyl, 2,4-bis(fluoromethoxy)phenyl, 2,4-bis(difluoromethoxy)phenyl, 2,4-bis(trifluoromethoxy)phenyl, 3,5-bis(fluoromethoxy)-phenyl, 3,5-bis(difluoromethoxy)phenyl, or 3,5-bis(trifluoromethoxy)phenyl. Exemplary embodiments of the present invention include a compound of Formula (I) wherein R¹ is 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, or 4-(N,N-diethylamino)phenyl.

In the present invention, R² is hydrogen, optionally substituted C₁-C₆ linear or branched alkyl, optionally substituted C₃-C₆ cycloalkyl, or —C(O)R²³ wherein R²³ is optionally substituted C₁-C₆ linear or branched alkyl or optionally substituted C₃-C₆ cycloalkyl.

Compounds of the present invention include those wherein R² is hydrogen, optionally substituted C₁-C₄ linear or branched alkyl, optionally substituted C₃-C₄ cycloalkyl, or —C(O)R²³ wherein R²³ is optionally substituted C₁-C₆ linear or branched alkyl or optionally substituted C₃-C₆ cycloalkyl.

Exemplary embodiments of the present invention include a compound of Formula (I) or a pharmaceutically acceptable salt form thereof wherein R² is optionally substituted C₁-C₄ linear or branched alkyl, optionally substituted C₃-C₄ cycloalkyl or —C(O)R²³ wherein R²³ is optionally substituted C₁-C₆ linear or branched alkyl or optionally substituted C₃-C₆ cycloalkyl.

Compounds of the present invention include those wherein R² is not hydrogen.

Exemplary embodiments of the present invention include a compound of Formula (I) or a pharmaceutically acceptable salt form thereof wherein R² is C₃-C₄ cycloalkyl or C₁-C₄ linear or branched alkyl optionally substituted with C₃-C₆ cycloalkyl.

Exemplary embodiments of the present invention include a compound of Formula (I) or a pharmaceutically acceptable salt form thereof wherein R² is methyl, ethyl, n-propyl, isopropyl, cyclopropyl, n-butyl, iso-butyl, sec-butyl, tert-butyl, or cyclopropylmethyl.

Compounds of the present invention include those wherein R³ is selected from:

i) hydrogen;

ii) optionally substituted C₁-C₆ linear or branched alkyl or optionally substituted C₃-C₆ cycloalkyl;

iii) —C(O)R⁴;

wherein R⁴ is optionally substituted C₁-C₆ linear or branched alkyl, optionally substituted C₃-C₆ cycloalkyl, optionally substituted heterocycle, optionally substituted aryl, or optionally substituted heteroaryl;
   iv) —C(O)NR⁵R⁶;
wherein R⁵ and R⁶ are each independently selected from
a) hydrogen;
b) optionally substituted C₁-C₆ linear or branched alkyl;
c) optionally substituted C₃-C₇ cycloalkyl;
d) —OR⁷;
   wherein R⁷ is hydrogen or optionally substituted C₁-C₆ linear or branched alkyl;
e) —NR⁸R⁹;
   wherein R⁸ and R⁹ are each independently hydrogen, optionally substituted C₁-C₆ linear or branched alkyl, optionally substituted C₁-C₆ linear or branched alkoxy, —OH, or —CO₂R¹⁰, wherein R¹⁰ is optionally substituted C₁-C₆ linear or branched alkyl; or R⁸ and R⁹ can be taken together with the atom to which they are bound to form an optionally substituted ring having from 3 to 7 ring atoms and optionally containing one or more additional heteroatom ring atoms independently selected from N, O, or S; or
f) R⁵ and R⁶ can be taken together with the atom to which they are bound to form an optionally substituted ring having from 3 to 7 ring atoms and optionally containing one or more additional heteroatom ring atoms independently selected from N, O, or S;
   v) —C(NR¹¹)R¹²;
wherein R¹¹ is
a) hydrogen;
b) optionally substituted C₁-C₆ linear or branched alkyl or optionally substituted C₃-C₆ cycloalkyl;
c) —OH; or
d) —CN;
and R¹² is
a) optionally substituted C₁-C₆ linear or branched alkyl or optionally substituted C₃-C₆ cycloalkyl;
b) —OR¹³;
   R¹³ is hydrogen, optionally substituted C₁-C₆ linear or branched alkyl, optionally substituted C₃-C₆ cycloalkyl, or optionally substituted aryl; or
c) —NR¹⁴R¹⁵;
   R¹⁴ and R¹⁵ are each independently hydrogen, optionally substituted aryl, optionally substituted C₁-C₆ linear or branched alkyl, or optionally substituted C₃-C₈ cycloalkyl;
      vi) —SO₂R¹⁶;
wherein R¹⁶ is optionally substituted aryl, optionally substituted C₁-C₆ linear or branched alkyl, or optionally substituted C₃-C₆ cycloalkyl;
   vii) —C(O)R¹⁷;
wherein R¹⁷ is optionally substituted aryl or optionally substituted heteroaryl;
   viii) —C(O)OR¹⁸;
wherein R¹⁸ is optionally substituted C₁-C₆ linear or branched alkyl, optionally substituted C₃-C₆ cycloalkyl, or optionally substituted aryl.

Exemplary embodiments of the present invention include a compound of Formula (I) or a pharmaceutically acceptable salt form thereof wherein R³ is selected from:

i) hydrogen;

ii) C₁-C₆ linear or branched alkyl or C₃-C₆ cycloalkyl;

iii) —C(O)R⁴;

wherein R⁴ is C₁-C₆ linear or branched alkyl or C₃-C₆ cycloalkyl;
   iv) —C(O)NR⁵R⁶;
wherein R⁵ and R⁶ are each independently selected from
a) hydrogen;
b) C₁-C₆ linear or branched alkyl;
c) C₃-C₇ cyclic alkyl;
d) —OR⁷;
   wherein R⁷ is hydrogen or C₁-C₆ linear or branched alkyl;
e) —NR⁸R⁹;
   wherein R⁸ and R⁹ are each independently hydrogen, C₁-C₆ linear or branched alkyl, C₁-C₆ linear or branched alkoxy, —OH, —CO₂R¹⁰ wherein R¹⁰ is C₁-C₆ linear or branched alkyl or R⁸ and R⁹ can be taken together to form a ring having from 3 to 7 ring atoms; or
f) R⁵ and R⁶ can be taken together to form a ring having from 3 to 7 ring atoms;
   v) —C(NR¹¹)R¹²;
wherein R¹¹ is
a) hydrogen;
b) C₁-C₆ linear or branched alkyl or C₃-C₆ cycloalkyl;
c) —OH; or
d) —CN;
and R¹² is
a) C₁-C₆ linear or branched alkyl;
b) —OR¹³;
   R¹³ is hydrogen, C₁-C₆ linear or branched alkyl, or phenyl; or
c) —NR¹⁴R¹⁵;
   R¹⁴ and R¹⁵ are each independently hydrogen, or C₁-C₆ linear or branched alkyl;
      vi) —SO₂R¹⁶;
wherein R¹⁶ is phenyl; or C₁-C₆ linear or branched alkyl;
   vii) —C(O)R¹⁷;
wherein R¹⁷ is C₁-C₅ heteroaryl;
   viii) —C(O)OR¹⁷;
wherein R¹⁸ is C₁-C₆ linear or branched alkyl;

In some embodiments, R³ is hydrogen.

In other embodiments, R³ is optionally substituted C₁-C₆ linear or branched alkyl or optionally substituted C₃-C₆ cycloalkyl. Examples of R³ include, but are not limited to, methyl, ethyl, n-propyl, iso-propyl, cyclopropyl, n-butyl, sec-butyl, iso-butyl, tert-butyl, cyclobutyl, n-pentyl, 2-methylbutyl, 3-methylbutyl, 1,1-dimethylpropyl, 2,2-dimethylpropyl, cyclopentyl, n-hexyl, 1-methylpentyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, and cyclohexyl.

In some embodiments, R³ is —C(O)R⁴, wherein R⁴ is optionally substituted C₁-C₆ linear or branched alkyl, optionally substituted C₃-C₆ cycloalkyl or optionally substituted heterocycle. Nonlimiting examples of R⁴ include methyl, ethyl, n-propyl, iso-propyl, cyclopropyl, n-butyl, sec-butyl, iso-butyl, tert-butyl, cyclobutyl, n-pentyl, 2-methylbutyl, 3-methylbutyl, 1,1-dimethylpropyl, 2,2-dimethylpropyl, cyclopentyl, n-hexyl, 1-methylpentyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, or cyclohexyl. Non-limiting examples of R³ include —C(O)CH₃, —C(O)cyclopropyl, and —C(O)CH₂cyclopropyl.

In other embodiments, R³ is —C(O)NR⁵R⁶, wherein each R⁵ and R⁶ are each independently selected from:

a) hydrogen;

b) optionally substituted C₁-C₆ linear or branched alkyl;

c) optionally substituted C₃-C₇ cycloalkyl;

d) —OR⁷;
wherein R⁷ is hydrogen or optionally substituted C₁-C₆ linear or branched alkyl;

e) —NR⁸R⁹;
wherein R⁸ and R⁹ are each independently hydrogen, optionally substituted C₁-C₆ linear or branched alkyl, optionally substituted C₁-C₆ linear or branched alkoxy, —OH, or —CO₂R¹⁰, wherein R¹⁰ is optionally substituted C₁-C₆ linear or branched alkyl; or R⁸ and R⁹ can be taken together with the atom to which they are bound to form an optionally substituted ring having from 3 to 7 ring atoms and optionally containing one or more additional heteroatom ring atoms independently selected from N, O, or S; or

f) R⁵ and R⁶ can be taken together with the atom to which they are bound to form an optionally substituted ring having from 3 to 7 ring atoms and optionally containing one or more additional heteroatom ring atoms independently selected from N, O, or S.

In exemplary embodiments of the present invention, R³ is C(O)NR⁵R⁶ wherein each R⁵ and R⁶ are each independently selected from

a) hydrogen;

b) C₁-C₆ linear or branched alkyl;

c) C₃-C₇ cyclic alkyl;

d) —OR⁷;
R⁷ is hydrogen or C₁-C₆ linear or branched alkyl;

e) —NR⁸R⁹;
R⁸ and R⁹ are each independently hydrogen, C₁-C₆ linear or branched alkyl, C₁-C₆ linear or branched alkoxy, —OH or —CO₂R¹, wherein R¹⁰ is C₁-C₆ linear or branched alkyl; or R⁸ and R⁹ can be taken together to from a ring having from 3 to 7 ring atoms; or

f) R⁵ and R⁶ can be taken together to form an optionally substituted ring having from 3 to 7 ring atoms.

Exemplary compounds of the invention include those wherein R⁵ and R⁶ are each independently selected from hydrogen, optionally substituted C₁-C₆ linear or branched alkyl, or optionally substituted C₃-C₇ cycloalkyl. Nonlimiting examples include methyl, ethyl, n-propyl, iso-propyl, cyclopropyl, n-butyl, sec-butyl, iso-butyl, tert-butyl, cyclobutyl, cyclopentyl, cyclohexyl, and cycloheptyl. Nonlimiting examples of R³ include —C(O)NH₂, —C(O)NHCH₃, —C(O)NHCH₂CH₃, —C(O)N(CH₂CH₃)₂, —C(O)N(CH₃)₂, and —C(O)NH[CH(CH₃)₂].

In some embodiments, R³ is —C(O)NR⁵R⁶ and R⁵ is —OR⁷ or —NR⁸R⁹; thereby forming R³ units having the formula —C(O)NR⁶OR⁷ or —C(O)NR⁶NR⁸R⁹, wherein, in exemplary embodiments, R⁶ is hydrogen, methyl, ethyl, n-propyl, iso-propyl, n-butyl, sec-butyl, iso-butyl, or tert-butyl; R⁷ is hydrogen, methyl, ethyl, n-propyl, iso-propyl, n-butyl, sec-butyl, iso-butyl, or tert-butyl; R⁸ and R⁹ are each independently hydrogen, methyl, ethyl, n-propyl, iso-propyl, n-butyl, sec-butyl, iso-butyl, tert-butyl, methoxy, ethoxy, n-propoxy, iso-propoxy, n-butoxy, sec-butoxy, iso-butoxy, or tert-butoxy; or R⁸ is —CO₂R¹⁰, R⁹ is hydrogen and R¹⁰ is optionally substituted C₁-C₆ linear or branched alkyl, non-limiting examples of which include methyl, ethyl, n-propyl, iso-propyl, n-butyl, sec-butyl, iso-butyl, and tert-butyl. Nonlimiting examples of R³ are —C(O)NHOH, —C(O)NHOCH₃, —C(O)NHNH₂, —C(O)NHOCH₂CH₃, —C(O)NCH₃OCH₃, —C(O)NHNHC(O)OCH₃, or —C(O)NHNHC(O)OC(CH₃)₃.

In some embodiments, R³ is —C(O)NR⁵R⁶ and R⁵ and R⁶ are taken together with the nitrogen to which they are attached to form an optionally substituted ring having from 3 to 7 ring atoms. Nonlimiting examples of rings formed from R⁵ and

R⁶ include aziridinyl, azetidinyl, pyrrolidinyl, piperazinyl, 4-methylpiperazinyl, morpholinyl, and piperidin-1-yl.

In some embodiments, R³ is —C(NR¹¹)R¹² wherein R¹¹ is hydrogen; optionally substituted C₁-C₆ linear or branched alkyl or optionally substituted C₃-C₆ cycloalkyl; hydroxyl (—OH); or cyano (—CN); and R¹² is optionally substituted C₁-C₆ linear or branched alkyl or optionally substituted C₃-C₆ cycloalkyl; —OR¹³, wherein R¹³ is hydrogen, optionally substituted aryl, optionally substituted C₁-C₆ linear or branched alkyl or optionally substituted C₃-C₆ cycloalkyl; or —NR¹⁴R¹⁵, wherein R¹⁴ and R¹⁵ are each independently hydrogen, optionally substituted aryl, optionally substituted C₁-C₆ linear or branched alkyl or optionally substituted C₃-C₆ cycloalkyl. Nonlimiting examples of R³ include —C(NCN)NH₂, —C(NCN)NHCH₃, and —C(NCN)NHC₆H₅.

Non-limiting examples of the alkyl groups of R¹¹, R¹², and R¹³, include methyl, ethyl, n-propyl, iso-propyl, cyclopropyl, n-butyl, sec-butyl, iso-butyl, and tert-butyl. Non-limiting examples of R¹⁴ and R¹⁵ groups include methyl, ethyl, n-propyl, iso-propyl, cyclopropyl, n-butyl, sec-butyl, iso-butyl, tert-butyl, and phenyl.

In some embodiments, R³ is —SO₂R¹⁶ wherein R¹⁶ is optionally substituted aryl (e.g., optionally substituted phenyl), optionally substituted C₁-C₆ linear or branched alkyl, or optionally substituted C₃-C₆ cycloalkyl. Non-limiting examples of R¹⁶ groups include methyl, ethyl, n-propyl, iso-propyl, cyclopropyl, n-butyl, sec-butyl, iso-butyl, tert-butyl, and phenyl. Nonlimiting examples of R³ include —SO₂CH₃, —SO₂C₆H₅, —SO₂CH₂CH₃, and —SO₂CH(CH₃)₂.

In some embodiments, R³ is —C(O)R¹⁷ wherein R¹⁷ is optionally substituted aryl or optionally substituted heteroaryl. Non-limiting examples include imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl, triazinyl, thiazol-2-yl, and thiazol-4-yl.

In some embodiments, R³ is —C(O)OR¹⁸ wherein R¹⁸ is optionally substituted C₁-C₆ linear or branched alkyl, optionally substituted C₃-C₆ cycloalkyl, or optionally substituted aryl. Non-limiting examples of R¹⁸ groups include methyl, ethyl, n-propyl, iso-propyl, cyclopropyl, n-butyl, sec-butyl, iso-butyl, and tert-butyl. Nonlimiting examples of R³ include —C(O)OCH₃, —C(O)OCH₂CH₃, —C(O)OCH(CH₃)₂, and —C(O)OC(CH₃)₃.

Exemplary embodiments of the present invention include a compound of Formula I or a pharmaceutically acceptable salt form thereof wherein R³ is hydrogen, —C(O)R⁴; —C(O)NR⁵R⁶, —C(O)NR⁵OR⁷; —C(O)NR⁵NR⁸R⁹, —C(NR¹¹)R¹², —SO₂R¹⁶, —C(O)OR¹⁸, or —C(O)R¹⁷; R⁴ is —CH₃, —CH₂CH₃, —CH₂CH₂CH₃, —CH(CH₃)₂, cyclopropyl, cyclobutyl, cyclopentyl, or cyclohexyl; R⁵ is hydrogen, —CH₃, —CH₂CH₃, or —CH(CH₃)₂; R⁶ is hydrogen, —CH₃, or —CH₂CH₃; or R⁵ and R⁶ are taken together to form aziridin-1-yl, pyrrolidin-1-yl, piperidin-1-yl, 4-(methyl)piperazin-1-yl, morpholin-4-yl; R⁷ is hydrogen, —CH₃, or —CH₂CH₃; R⁸ is hydrogen; R⁹ is hydrogen, —C(O)OCH₃, or —C(O)OC(CH₃)₃; R¹¹ is OH, or —CN; R¹²—NH₂, —CH₃, or —NR⁴R⁵; R¹⁴ is hydrogen, CH₃, or phenyl; R¹⁵ is hydrogen, CH₃, or phenyl; R¹⁶ is —CH₃, —CH₂CH₃, —CH(CH₃)₂, or —C₆H₅; R¹⁸ is —CH₃, —CH₂CH₃, —CH(CH₃)₂, —C₆H₅, or —C(CH₃)₃; and R¹⁷ is imidazolin-1-yl, isoxazolin-5-yl, furan-2-yl, thiophen-2-yl, azetidin-1-yl, pyrrolidin-1-yl, piperidin-1-yl, piperazin-1-yl, or morpholin-4-yl.

Exemplary embodiments of the present invention include a compound of Formula I or a pharmaceutically acceptable salt form thereof wherein R³ is hydrogen, —C(O)CH₃, —C(O)cyclopropyl, —C(O)NH₂, —C(O)NHCH₃, —C(O)N(CH₃)₂, —C(O)NH[CH(CH₃)₂], —C(O)NHCH₂CH₃, —C(O)N(CH₂CH₃), —C(O)OCH₃, —C(O)OCH₂CH₃, —C(O)OCH(CH₃)₂, —C(O)OC(CH₃)₃, —C(O)NHOH, —C(O)NHOCH₃, —C(O)N(CH₃)OCH₃, —C(O)NHNH₂, —C(O)NHOCH₂CH₃, —C(O)NCH₃OCH₃, —C(O)NHNHC(O)OCH₃, —C(O)NHNHC(O)OC(CH₃)₃, —C(NCN)NH₂, —C(NCN)NHCH₃, —C(NCN)NHC₆H₅, —C(O)aziridin-1-yl, —C(O)azetidin-1-yl, —C(O)pyrrolidin-1-yl, —C(O)piperidin-1-yl, —C(O)piperazin-1-yl, —C(O)morpholin-4-yl, —C(O)imidazolin-1-yl, —C(O)isoxazolin-5-yl, —SO₂CH₃, —SO₂CH₂CH₃, —SO₂CH(CH₃)₂, or SO₂C₆H₅.

In all of the embodiments provided herein, examples of suitable optional substituents are not intended to limit the scope of the claimed invention. The compounds of the invention may contain any of the substituents, or combinations of substituents, provided herein.

L, L¹, and L² are linking units each independently having the formula:
—[C(R¹⁹)₂]ₙ—
each R¹⁹ unit present in a linking unit is independently chosen from hydrogen, methyl, or ethyl; n is 1 to 4; x, y, and z are each independently 0 or 1. When x is equal to 0, linking group L is absent, and when x is equal to 1, linking group L is present. Likewise, when y is equal to 0, linking group L¹ is absent, and when y is equal to 1, linking group L¹ is present. In addition, when z is equal to 0, linking group L² is absent, and when z is equal to 1, linking group L² is present.

Exemplary embodiments of the present invention include a compound of Formula (I) or a pharmaceutically acceptable salt form thereof wherein x is 1 and L is —CH₂CH₂— (ethylene). Compounds according to these embodiments have the formula (II) or a pharmaceutically acceptable salt form thereof: 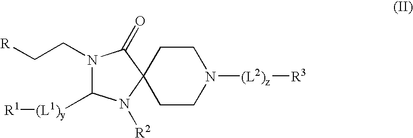
wherein R¹, R², R³, R¹, L¹, L², y, and z are the same as defined herein.

Exemplary embodiments of the present invention include a compound of Formula (I) or a pharmaceutically acceptable salt form thereof wherein x is 1 and L is —CH₂— (methylene). Compounds according to these embodiments have the formula (III) or a pharmaceutically acceptable salt form thereof: 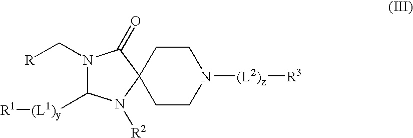
wherein R¹, R², R³, R¹, L¹ L², y, and z are the same as defined herein.

Exemplary embodiments of the present invention include a compound of Formula (I) or a pharmaceutically acceptable salt form thereof wherein y is 0 and the compounds have the formula (IV) or a pharmaceutically acceptable salt form thereof: 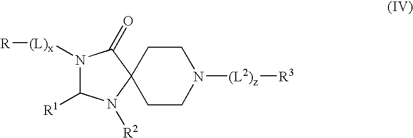
wherein R¹, R², R³, R², L, L², x, and z are the same as defined herein.

Exemplary embodiments of the present invention include a compound of Formula (I) or a pharmaceutically acceptable salt form thereof wherein y is 1 and L¹ is —CH₂— (methylene). Compounds according to these embodiments have the formula (V) or a pharmaceutically acceptable salt form thereof: 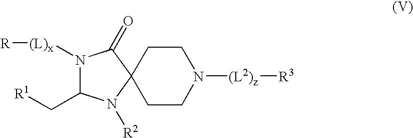
wherein R¹, R², R³, R², L, L², x, and z are the same as defined herein.

Exemplary embodiments of the present invention include a compound of Formula (I) or a pharmaceutically acceptable salt form thereof wherein z is 0 and the compounds have the formula (VI) or a pharmaceutically acceptable salt form thereof: 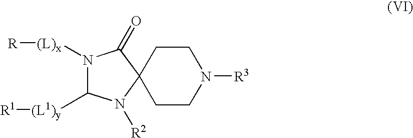
wherein R¹, R², R³, R¹, L, L², x, and y are the same as defined herein

Exemplary embodiments of the present invention include a compound of Formula (I) or a pharmaceutically acceptable salt form thereof wherein z is 1 and L² is —CH₂— (methylene). Compounds according the these embodiments have the formula (VII) or a pharmaceutically acceptable salt form thereof:

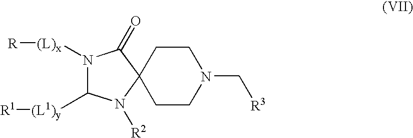

As it relates to the Kv1.5 potassium channel inhibitors of the present invention the linking units L, L¹, and L² may be present or absent in any combination. For example, in some compounds according to the invention, x is 1, y is 0 and z is 0; in other embodiments, x is 1, y is 0 and z is 1; in still other embodiments, x is 1, y is 1 and z is 0.

The skilled practitioner will understand that combinations of the embodiments provided herein are encompassed within the scope of the present invention.

Compounds of the present invention include 4-oxo-1,3,8-triaza-spiro[4.5]decanes having the formula (VIII) or a pharmaceutically acceptable salt form thereof:

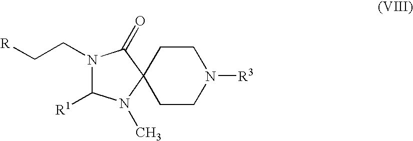

Compounds of the present invention include compounds having the formula (IX) or a pharmaceutically acceptable salt form thereof:

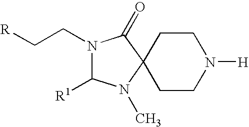

wherein non-limiting examples of R and R¹ are defined herein below in Table I.

TABLE I

| R | R¹ |
|---|---|
| 4-methoxyphenyl | 4-tert-butylphenyl |
| 4-methoxyphenyl | 4-trifluoromethylphenyl |
| 4-methoxyphenyl | 4-cyclopropylphenyl |
| 4-methoxyphenyl | 4-diethylaminophenyl |
| 4-methoxyphenyl | 4-difluoromethoxyphenyl |
| 4-trifluoromethylphenyl | 4-tert-butylphenyl |
| 4-trifluoromethylphenyl | 4-trifluoromethylphenyl |
| 4-trifluoromethylphenyl | 4-cyclopropylphenyl |
| 4-trifluoromethylphenyl | 4-diethylaminophenyl |
| 4-trifluoromethylphenyl | 4-difluoromethoxyphenyl |
| 4-cyclopropylphenyl | 4-tert-butylphenyl |
| 4-cyclopropylphenyl | 4-trifluoromethylphenyl |
| 4-cyclopropylphenyl | 4-cyclopropylphenyl |
| 4-cyclopropylphenyl | 4-diethylaminophenyl |
| 4-cyclopropylphenyl | 4-difluoromethoxyphenyl |
| 4-diethylaminophenyl | 4-tert-butylphenyl |
| 4-diethylaminophenyl | 4-trifluoromethylphenyl |
| 4-diethylaminophenyl | 4-cyclopropylphenyl |
| 4-diethylaminophenyl | 4-diethylaminophenyl |
| 4-diethylaminophenyl | 4-difluoromethoxyphenyl |
| 4-difluoromethoxyphenyl | 4-tert-butylphenyl |
| 4-difluoromethoxyphenyl | 4-trifluoromethylphenyl |
| 4-difluoromethoxyphenyl | 4-cyclopropylphenyl |
| 4-difluoromethoxyphenyl | 4-diethylaminophenyl |
| 4-difluoromethoxyphenyl | 4-difluoromethoxyphenyl |

Compounds of the present invention include compounds having the formula (X) or a pharmaceutically acceptable salt form thereof:

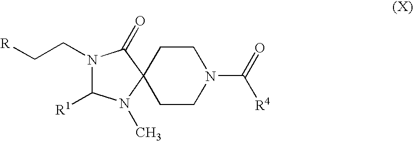

wherein non-limiting examples of R, R¹, and R⁴ are defined herein below in Table II.

TABLE II

| R | R¹ | R⁴ |
|---|---|---|
| 4-methoxyphenyl | 4-tert-butylphenyl | —CH₃ |
| 4-methoxyphenyl | 4-trifluoromethylphenyl | —CH₃ |
| 4-methoxyphenyl | 4-cyclopropylphenyl | —CH₃ |
| 4-methoxyphenyl | 4-diethylaminophenyl | —CH₃ |
| 4-methoxyphenyl | 4-difluoromethoxyphenyl | —CH₃ |
| 4-methoxyphenyl | 4-tert-butylphenyl | —CH₂CH₃ |
| 4-methoxyphenyl | 4-trifluoromethylphenyl | —CH₂CH₃ |
| 4-methoxyphenyl | 4-cyclopropylphenyl | —CH₂CH₃ |
| 4-methoxyphenyl | 4-diethylaminophenyl | —CH₂CH₃ |
| 4-methoxyphenyl | 4-difluoromethoxyphenyl | —CH₂CH₃ |
| 4-methoxyphenyl | 4-tert-butylphenyl | —CH₂CH₂CH₃ |
| 4-methoxyphenyl | 4-trifluoromethylphenyl | —CH₂CH₂CH₃ |
| 4-methoxyphenyl | 4-cyclopropylphenyl | —CH₂CH₂CH₃ |
| 4-methoxyphenyl | 4-diethylaminophenyl | —CH₂CH₂CH₃ |
| 4-methoxyphenyl | 4-difluoromethoxyphenyl | —CH₂CH₂CH₃ |
| 4-methoxyphenyl | 4-tert-butylphenyl | —CH(CH₃)₂ |
| 4-methoxyphenyl | 4-trifluoromethylphenyl | —CH(CH₃)₂ |
| 4-methoxyphenyl | 4-cyclopropylphenyl | —CH(CH₃)₂ |
| 4-methoxyphenyl | 4-diethylaminophenyl | —CH(CH₃)₂ |
| 4-methoxyphenyl | 4-difluoromethoxyphenyl | —CH(CH₃)₂ |
| 4-methoxyphenyl | 4-tert-butylphenyl | -cyclopropyl |
| 4-methoxyphenyl | 4-trifluoromethylphenyl | -cyclopropyl |
| 4-methoxyphenyl | 4-cyclopropylphenyl | -cyclopropyl |
| 4-methoxyphenyl | 4-diethylaminophenyl | -cyclopropyl |
| 4-methoxyphenyl | 4-difluoromethoxyphenyl | -cyclopropyl |
| 4-methoxyphenyl | 4-tert-butylphenyl | -cyclobutyl |
| 4-methoxyphenyl | 4-trifluoromethylphenyl | -cyclobutyl |
| 4-methoxyphenyl | 4-cyclopropylphenyl | -cyclobutyl |
| 4-methoxyphenyl | 4-diethylaminophenyl | -cyclobutyl |
| 4-methoxyphenyl | 4-difluoromethoxyphenyl | -cyclobutyl |
| 4-methoxyphenyl | 4-tert-butylphenyl | -cyclopentyl |
| 4-methoxyphenyl | 4-trifluoromethylphenyl | -cyclopentyl |
| 4-methoxyphenyl | 4-cyclopropylphenyl | -cyclopentyl |
| 4-methoxyphenyl | 4-diethylaminophenyl | -cyclopentyl |
| 4-methoxyphenyl | 4-difluoromethoxyphenyl | -cyclopentyl |
| 4-methoxyphenyl | 4-tert-butylphenyl | -cyclohexyl |
| 4-methoxyphenyl | 4-trifluoromethylphenyl | -cyclohexyl |
| 4-methoxyphenyl | 4-cyclopropylphenyl | -cyclohexyl |
| 4-methoxyphenyl | 4-diethylaminophenyl | -cyclohexyl |
| 4-methoxyphenyl | 4-difluoromethoxyphenyl | -cyclohexyl |

Compounds of the present invention include compounds having the formula (XI) or a pharmaceutically acceptable salt form thereof:

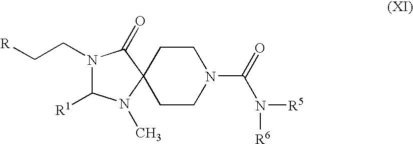

wherein non-limiting examples of R, R¹, R⁵, and R⁶ are defined herein below in Table III.

TABLE III

| R | R¹ | R⁵ | R⁶ |
|---|---|---|---|
| 4-methoxyphenyl | 4-tert-butylphenyl | —H | —H |
| 4-methoxyphenyl | 4-trifluoromethylphenyl | —H | —H |
| 4-methoxyphenyl | 4-cyclopropylphenyl | —H | —H |
| 4-methoxyphenyl | 4-diethylaminophenyl | —H | —H |
| 4-methoxyphenyl | 4-difluoromethoxyphenyl | —H | —H |
| 4-methoxyphenyl | 4-tert-butylphenyl | —CH₃ | —H |
| 4-methoxyphenyl | 4-trifluoromethylphenyl | —CH₃ | —H |
| 4-methoxyphenyl | 4-cyclopropylphenyl | —CH₃ | —H |
| 4-methoxyphenyl | 4-diethylaminophenyl | —CH₃ | —H |
| 4-methoxyphenyl | 4-difluoromethoxyphenyl | —CH₃ | —H |
| 4-methoxyphenyl | 4-tert-butylphenyl | —CH₃ | —CH₃ |
| 4-methoxyphenyl | 4-trifluoromethylphenyl | —CH₃ | —CH₃ |
| 4-methoxyphenyl | 4-cyclopropylphenyl | —CH₃ | —CH₃ |
| 4-methoxyphenyl | 4-diethylaminophenyl | —CH₃ | —CH₃ |
| 4-methoxyphenyl | 4-difluoromethoxyphenyl | —CH₃ | —CH₃ |
| 4-methoxyphenyl | 4-tert-butylphenyl | —CH₂CH₃ | —H |
| 4-methoxyphenyl | 4-trifluoromethylphenyl | —CH₂CH₃ | —H |
| 4-methoxyphenyl | 4-cyclopropylphenyl | —CH₂CH₃ | —H |
| 4-methoxyphenyl | 4-diethylaminophenyl | —CH₂CH₃ | —H |
| 4-methoxyphenyl | 4-difluoromethoxyphenyl | —CH₂CH₃ | —H |
| 4-methoxyphenyl | 4-tert-butylphenyl | —CH₂CH₃ | —CH₂CH₃ |
| 4-methoxyphenyl | 4-trifluoromethylphenyl | —CH₂CH₃ | —CH₂CH₃ |
| 4-methoxyphenyl | 4-cyclopropylphenyl | —CH₂CH₃ | —CH₂CH₃ |
| 4-methoxyphenyl | 4-diethylaminophenyl | —CH₂CH₃ | —CH₂CH₃ |
| 4-methoxyphenyl | 4-difluoromethoxyphenyl | —CH₂CH₃ | —CH₂CH₃ |
| 4-methoxyphenyl | 4-tert-butylphenyl | —CH(CH₃)₂ | —H |
| 4-methoxyphenyl | 4-trifluoromethylphenyl | —CH(CH₃)₂ | —H |
| 4-methoxyphenyl | 4-cyclopropylphenyl | —CH(CH₃)₂ | —H |
| 4-methoxyphenyl | 4-diethylaminophenyl | —CH(CH₃)₂ | —H |
| 4-methoxyphenyl | 4-difluoromethoxyphenyl | —CH(CH₃)₂ | —H |

| R | R¹ | R⁵ and R⁶ taken together |
|---|---|---|
| 4-methoxyphenyl | 4-tert-butylphenyl | aziridin-1-yl |
| 4-methoxyphenyl | 4-trifluoromethylphenyl | aziridin-1-yl |
| 4-methoxyphenyl | 4-cyclopropylphenyl | aziridin-1-yl |
| 4-methoxyphenyl | 4-diethylaminophenyl | aziridin-1-yl |
| 4-methoxyphenyl | 4-difluoromethoxyphenyl | aziridin-1-yl |
| 4-methoxyphenyl | 4-tert-butylphenyl | azetidin-1-yl |
| 4-methoxyphenyl | 4-trifluoromethylphenyl | azetidin-1-yl |
| 4-methoxyphenyl | 4-cyclopropylphenyl | azetidin-1-yl |
| 4-methoxyphenyl | 4-diethylaminophenyl | azetidin-1-yl |
| 4-methoxyphenyl | 4-difluoromethoxyphenyl | azetidin-1-yl |
| 4-methoxyphenyl | 4-tert-butylphenyl | pyrrolidin-1-yl |
| 4-methoxyphenyl | 4-trifluoromethylphenyl | pyrrolidin-1-yl |
| 4-methoxyphenyl | 4-cyclopropylphenyl | pyrrolidin-1-yl |
| 4-methoxyphenyl | 4-diethylaminophenyl | pyrrolidin-1-yl |
| 4-methoxyphenyl | 4-difluoromethoxyphenyl | pyrrolidin-1-yl |
| 4-methoxyphenyl | 4-tert-butylphenyl | piperidin-1-yl |
| 4-methoxyphenyl | 4-trifluoromethylphenyl | piperidin-1-yl |
| 4-methoxyphenyl | 4-cyclopropylphenyl | piperidin-1-yl |
| 4-methoxyphenyl | 4-diethylaminophenyl | piperidin-1-yl |
| 4-methoxyphenyl | 4-difluoromethoxyphenyl | piperidin-1-yl |
| 4-methoxyphenyl | 4-tert-butylphenyl | piperazin-1-yl |
| 4-methoxyphenyl | 4-trifluoromethylphenyl | piperazin-1-yl |
| 4-methoxyphenyl | 4-tert-butylphenyl | piperazin-1-yl |
| 4-methoxyphenyl | 4-trifluoromethylphenyl | piperazin-1-yl |
| 4-methoxyphenyl | 4-cyclopropylphenyl | piperazin-1-yl |
| 4-methoxyphenyl | 4-diethylaminophenyl | 4-(methyl)piperazin-1-yl |
| 4-methoxyphenyl | 4-difluoromethoxyphenyl | 4-(methyl)piperazin-1-yl |
| 4-methoxyphenyl | 4-tert-butylphenyl | 4-(methyl)piperazin-1-yl |
| 4-methoxyphenyl | 4-trifluoromethylphenyl | 4-(methyl)piperazin-1-yl |
| 4-methoxyphenyl | 4-cyclopropylphenyl | 4-(methyl)piperazin-1-yl |
| 4-methoxyphenyl | 4-diethylaminophenyl | morpholin-4-yl |
| 4-methoxyphenyl | 4-difluoromethoxyphenyl | morpholin-4-yl |
| 4-methoxyphenyl | 4-tert-butylphenyl | morpholin-4-yl |
| 4-methoxyphenyl | 4-trifluoromethylphenyl | morpholin-4-yl |
| 4-methoxyphenyl | 4-cyclopropylphenyl | morpholin-4-yl |

Compounds of the present invention include compounds having the formulas (XII) or (XIII) or a pharmaceutically acceptable salt form thereof:

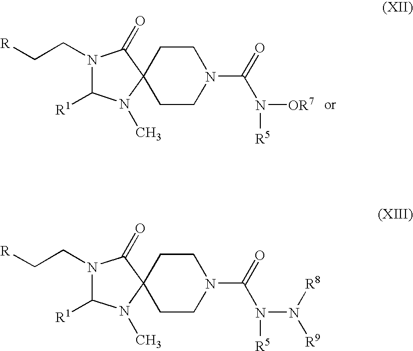

wherein non-limiting examples of R, R¹, R⁵, R⁷, R⁸ and R⁹ are defined herein below in Tables IV and V.

TABLE IV

| R | R¹ | R⁵ | R⁷ |
|---|---|---|---|
| 4-methoxyphenyl | 4-tert-butylphenyl | —H | —H |
| 4-methoxyphenyl | 4-trifluoromethylphenyl | —H | —H |
| 4-methoxyphenyl | 4-cyclopropylphenyl | —H | —H |
| 4-methoxyphenyl | 4-diethylaminophenyl | —H | —H |
| 4-methoxyphenyl | 4-difluoromethoxyphenyl | —H | —H |
| 4-methoxyphenyl | 4-tert-butylphenyl | —H | —CH₃ |
| 4-methoxyphenyl | 4-trifluoromethylphenyl | —H | —CH₃ |
| 4-methoxyphenyl | 4-cyclopropylphenyl | —H | —CH₃ |
| 4-methoxyphenyl | 4-diethylaminophenyl | —H | —CH₃ |
| 4-methoxyphenyl | 4-difluoromethoxyphenyl | —H | —CH₃ |
| 4-methoxyphenyl | 4-tert-butylphenyl | —H | —CH₂CH₃ |
| 4-methoxyphenyl | 4-trifluoromethylphenyl | —H | —CH₂CH₃ |
| 4-methoxyphenyl | 4-cyclopropylphenyl | —H | —CH₂CH₃ |
| 4-methoxyphenyl | 4-diethylaminophenyl | —H | —CH₂CH₃ |
| 4-methoxyphenyl | 4-difluoromethoxyphenyl | —H | —CH₂CH₃ |
| 4-methoxyphenyl | 4-tert-butylphenyl | —CH₃ | —CH₃ |
| 4-methoxyphenyl | 4-trifluoromethylphenyl | —CH₃ | —CH₃ |
| 4-methoxyphenyl | 4-cyclopropylphenyl | —CH₃ | —CH₃ |
| 4-methoxyphenyl | 4-diethylaminophenyl | —CH₃ | —CH₃ |
| 4-methoxyphenyl | 4-difluoromethoxyphenyl | —CH₃ | —CH₃ |

TABLE V

| R | R¹ | R⁵ | R⁸ | R⁹ |
|---|---|---|---|---|
| 4-methoxyphenyl | 4-tert-butylphenyl | —H | —H | —H |
| 4-methoxyphenyl | 4-trifluoromethylphenyl | —H | —H | —H |
| 4-methoxyphenyl | 4-cyclopropylphenyl | —H | —H | —H |
| 4-methoxyphenyl | 4-diethylaminophenyl | —H | —H | —H |
| 4-methoxyphenyl | 4-difluoromethoxyphenyl | —H | —H | —H |
| 4-methoxyphenyl | 4-tert-butylphenyl | —CH₃ | —H | —H |
| 4-methoxyphenyl | 4-trifluoromethylphenyl | —CH₃ | —H | —H |
| 4-methoxyphenyl | 4-cyclopropylphenyl | —CH₃ | —H | —H |
| 4-methoxyphenyl | 4-diethylaminophenyl | —CH₃ | —H | —H |
| 4-methoxyphenyl | 4-difluoromethoxyphenyl | —CH₃ | —H | —H |
| 4-methoxyphenyl | 4-tert-butylphenyl | —H | —H | —C(O)OCH₃ |
| 4-methoxyphenyl | 4-trifluoromethylphenyl | —H | —H | —C(O)OCH₃ |
| 4-methoxyphenyl | 4-cyclopropylphenyl | —H | —H | —C(O)OCH₃ |
| 4-methoxyphenyl | 4-diethylaminophenyl | —H | —H | —C(O)OCH₃ |
| 4-methoxyphenyl | 4-difluoromethoxyphenyl | —H | —H | —C(O)OCH₃ |
| 4-methoxyphenyl | 4-tert-butylphenyl | —H | —H | —C(O)OC(CH₃)₃ |
| 4-methoxyphenyl | 4-trifluoromethylphenyl | —H | —H | —C(O)OC(CH₃)₃ |
| 4-methoxyphenyl | 4-cyclopropylphenyl | —H | —H | —C(O)OC(CH₃)₃ |
| 4-methoxyphenyl | 4-diethylaminophenyl | —H | —H | —C(O)OC(CH₃)₃ |
| 4-methoxyphenyl | 4-difluoromethoxyphenyl | —H | —H | —C(O)OC(CH₃)₃ |

Compounds of the present invention include compounds having the formula (XIV) or a pharmaceutically acceptable salt form thereof:

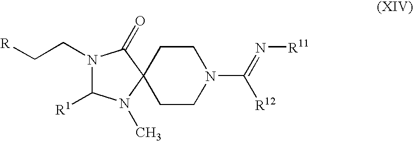

wherein non-limiting examples of R, R¹, R¹¹, and R¹² are defined herein below in Table VI.

TABLE VI

| R | R¹ | R¹¹ | R¹² |
|---|---|---|---|
| 4-methoxyphenyl | 4-tert-butylphenyl | —OH | —NH₂ |
| 4-methoxyphenyl | 4-trifluoromethylphenyl | —OH | —NH₂ |
| 4-methoxyphenyl | 4-cyclopropylphenyl | —OH | —NH₂ |
| 4-methoxyphenyl | 4-diethylaminophenyl | —OH | —NH₂ |
| 4-methoxyphenyl | 4-difluoromethoxyphenyl | —OH | —NH₂ |
| 4-methoxyphenyl | 4-tert-butylphenyl | —CN | —NH₂ |
| 4-methoxyphenyl | 4-trifluoromethylphenyl | —CN | —NH₂ |
| 4-methoxyphenyl | 4-cyclopropylphenyl | —CN | —NH₂ |
| 4-methoxyphenyl | 4-diethylaminophenyl | —CN | —NH₂ |
| 4-methoxyphenyl | 4-difluoromethoxyphenyl | —CN | —NH₂ |
| 4-methoxyphenyl | 4-tert-butylphenyl | —OH | —CH₃ |
| 4-methoxyphenyl | 4-trifluoromethylphenyl | —OH | —CH₃ |
| 4-methoxyphenyl | 4-cyclopropylphenyl | —OH | —CH₃ |
| 4-methoxyphenyl | 4-diethylaminophenyl | —OH | —CH₃ |
| 4-methoxyphenyl | 4-difluoromethoxyphenyl | —OH | —CH₃ |
| 4-methoxyphenyl | 4-tert-butylphenyl | —CN | —CH₃ |
| 4-methoxyphenyl | 4-trifluoromethylphenyl | —CN | —CH₃ |
| 4-methoxyphenyl | 4-cyclopropylphenyl | —CN | —CH₃ |
| 4-methoxyphenyl | 4-diethylaminophenyl | —CN | —CH₃ |
| 4-methoxyphenyl | 4-difluoromethoxyphenyl | —CN | —CH₃ |

Compounds of the present invention include compounds having the formula (XV) or a pharmaceutically acceptable salt form thereof:

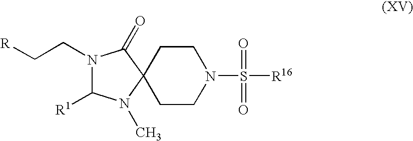

wherein non-limiting examples of R, R¹, and R¹⁶ are defined herein below in Table VII.

TABLE VII

| R | R¹ | R¹⁶ |
|---|---|---|
| 4-methoxyphenyl | 4-tert-butylphenyl | —CH₃ |
| 4-methoxyphenyl | 4-trifluoromethylphenyl | —CH₃ |
| 4-methoxyphenyl | 4-cyclopropylphenyl | —CH₃ |
| 4-methoxyphenyl | 4-diethylaminophenyl | —CH₃ |
| 4-methoxyphenyl | 4-difluoromethoxyphenyl | —CH₃ |
| 4-methoxyphenyl | 4-tert-butylphenyl | —CH₂CH₃ |
| 4-methoxyphenyl | 4-trifluoromethylphenyl | —CH₂CH₃ |
| 4-methoxyphenyl | 4-cyclopropylphenyl | —CH₂CH₃ |
| 4-methoxyphenyl | 4-diethylaminophenyl | —CH₂CH₃ |
| 4-methoxyphenyl | 4-difluoromethoxyphenyl | —CH₂CH₃ |
| 4-methoxyphenyl | 4-tert-butylphenyl | —CH(CH₃)₂ |
| 4-methoxyphenyl | 4-trifluoromethylphenyl | —CH(CH₃)₂ |
| 4-methoxyphenyl | 4-cyclopropylphenyl | —CH(CH₃)₂ |
| 4-methoxyphenyl | 4-diethylaminophenyl | —CH(CH₃)₂ |
| 4-methoxyphenyl | 4-difluoromethoxyphenyl | —CH(CH₃)₂ |
| 4-methoxyphenyl | 4-tert-butylphenyl | —C₆H₅ |
| 4-methoxyphenyl | 4-trifluoromethylphenyl | —C₆H₅ |
| 4-methoxyphenyl | 4-cyclopropylphenyl | —C₆H₅ |
| 4-methoxyphenyl | 4-diethylaminophenyl | —C₆H₅ |
| 4-methoxyphenyl | 4-difluoromethoxyphenyl | —C₆H₅ |

Compounds of the present invention include compounds having the formula (XVI) or a pharmaceutically acceptable salt form thereof:

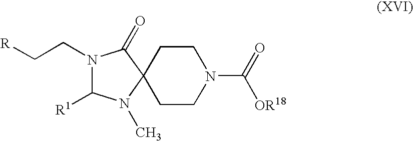

wherein nonlimiting examples of R, R¹, and R¹⁸ are defined herein below in Table VIII.

TABLE VIII

| R | R¹ | R¹⁸ |
|---|---|---|
| 4-methoxyphenyl | 4-tert-butylphenyl | —CH₃ |
| 4-methoxyphenyl | 4-trifluoromethylphenyl | —CH₃ |
| 4-methoxyphenyl | 4-cyclopropylphenyl | —CH₃ |
| 4-methoxyphenyl | 4-diethylaminophenyl | —CH₃ |
| 4-methoxyphenyl | 4-difluoromethoxyphenyl | —CH₃ |
| 4-methoxyphenyl | 4-tert-butylphenyl | —CH₂CH₃ |
| 4-methoxyphenyl | 4-trifluoromethylphenyl | —CH₂CH₃ |
| 4-methoxyphenyl | 4-cyclopropylphenyl | —CH₂CH₃ |
| 4-methoxyphenyl | 4-diethylaminophenyl | —CH₂CH₃ |
| 4-methoxyphenyl | 4-difluoromethoxyphenyl | —CH₂CH₃ |
| 4-methoxyphenyl | 4-tert-butylphenyl | —CH(CH₃)₂ |
| 4-methoxyphenyl | 4-trifluoromethylphenyl | —CH(CH₃)₂ |
| 4-methoxyphenyl | 4-cyclopropylphenyl | —CH(CH₃)₂ |
| 4-methoxyphenyl | 4-diethylaminophenyl | —CH(CH₃)₂ |
| 4-methoxyphenyl | 4-difluoromethoxyphenyl | —CH(CH₃)₂ |
| 4-methoxyphenyl | 4-tert-butylphenyl | —C₆H₅ |
| 4-methoxyphenyl | 4-trifluoromethylphenyl | —C₆H₅ |
| 4-methoxyphenyl | 4-cyclopropylphenyl | —C₆H₅ |
| 4-methoxyphenyl | 4-diethylaminophenyl | —C₆H₅ |
| 4-methoxyphenyl | 4-difluoromethoxyphenyl | —C₆H₅ |

Compounds of the present invention include compounds having the formula (XVII) or a pharmaceutically acceptable salt form thereof:

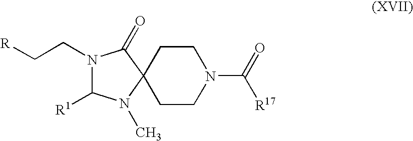

wherein non-limiting examples of R, R¹, and R¹⁷ are defined herein below in Table IX.

TABLE IX

| R | R¹ | R¹⁷ |
|---|---|---|
| 4-methoxyphenyl | 4-tert-butylphenyl | imidazolin-1-yl |
| 4-methoxyphenyl | 4-trifluoromethylphenyl | imidazolin-1-yl |
| 4-methoxyphenyl | 4-cyclopropylphenyl | imidazolin-1-yl |
| 4-methoxyphenyl | 4-diethylaminophenyl | imidazolin-1-yl |
| 4-methoxyphenyl | 4-difluoromethoxyphenyl | imidazolin-1-yl |
| 4-methoxyphenyl | 4-tert-butylphenyl | isoxazolin-5-yl |
| 4-methoxyphenyl | 4-trifluoromethylphenyl | isoxazolin-5-yl |
| 4-methoxyphenyl | 4-cyclopropylphenyl | isoxazolin-5-yl |
| 4-methoxyphenyl | 4-diethylaminophenyl | isoxazolin-5-yl |
| 4-methoxyphenyl | 4-difluoromethoxyphenyl | isoxazolin-5-yl |
| 4-methoxyphenyl | 4-tert-butylphenyl | furan-2-yl |
| 4-methoxyphenyl | 4-trifluoromethylphenyl | furan-2-yl |
| 4-methoxyphenyl | 4-cyclopropylphenyl | furan-2-yl |
| 4-methoxyphenyl | 4-diethylaminophenyl | furan-2-yl |
| 4-methoxyphenyl | 4-difluoromethoxyphenyl | furan-2-yl |
| 4-methoxyphenyl | 4-tert-butylphenyl | thiophen-2-yl |
| 4-methoxyphenyl | 4-trifluoromethylphenyl | thiophen-2-yl |
| 4-methoxyphenyl | 4-cyclopropylphenyl | thiophen-2-yl |
| 4-methoxyphenyl | 4-diethylaminophenyl | thiophen-2-yl |
| 4-methoxyphenyl | 4-difluoromethoxyphenyl | thiophen-2-yl |

Compounds of the present invention include 4-oxo-1,3,8-triaza-spiro[4.5]decanes having the formula (XVIII) or a pharmaceutically acceptable salt form thereof:

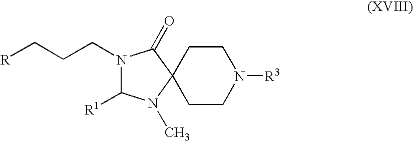

wherein nonlimiting examples of R, R¹ and R³ are defined herein below in Table X.

TABLE X

| R | R¹ | R³ |
|---|---|---|
| phenyl | 4-tert-butylphenyl | —H |
| phenyl | 4-trifluoromethylphenyl | —H |
| phenyl | 4-cyclopropylphenyl | —H |
| phenyl | 4-diethylaminophenyl | —H |
| phenyl | 4-difluoromethoxyphenyl | —H |
| phenyl | 4-tert-butylphenyl | —C(O)CH₃ |
| phenyl | 4-trifluoromethylphenyl | —C(O)CH₃ |
| phenyl | 4-cyclopropylphenyl | —C(O)CH₃ |
| phenyl | 4-diethylaminophenyl | —C(O)CH₃ |
| phenyl | 4-difluoromethoxyphenyl | —C(O)CH₃ |
| phenyl | 4-tert-butylphenyl | —C(O)cyclopropyl |
| phenyl | 4-trifluoromethylphenyl | —C(O)cyclopropyl |
| phenyl | 4-cyclopropylphenyl | —C(O)cyclopropyl |
| phenyl | 4-diethylaminophenyl | —C(O)cyclopropyl |
| phenyl | 4-difluoromethoxyphenyl | —C(O)cyclopropyl |
| phenyl | 4-tert-butylphenyl | —C(O)NH₂ |
| phenyl | 4-trifluoromethylphenyl | —C(O)NH₂ |
| phenyl | 4-cyclopropylphenyl | —C(O)NH₂ |
| phenyl | 4-diethylaminophenyl | —C(O)NH₂ |
| phenyl | 4-difluoromethoxyphenyl | —C(O)NH₂ |
| phenyl | 4-tert-butylphenyl | —C(O)NHCH₃ |
| phenyl | 4-trifluoromethylphenyl | —C(O)NHCH₃ |
| phenyl | 4-cyclopropylphenyl | —C(O)NHCH₃ |
| phenyl | 4-diethylaminophenyl | —C(O)NHCH₃ |
| phenyl | 4-difluoromethoxyphenyl | —C(O)NHCH₃ |
| phenyl | 4-tert-butylphenyl | —C(O)N(CH₃)₂ |
| phenyl | 4-trifluoromethylphenyl | —C(O)N(CH₃)₂ |
| phenyl | 4-cyclopropylphenyl | —C(O)N(CH₃)₂ |
| phenyl | 4-diethylaminophenyl | —C(O)N(CH₃)₂ |
| phenyl | 4-difluoromethoxyphenyl | —C(O)N(CH₃)₂ |
| phenyl | 4-tert-butylphenyl | —C(O)NCH(CH₃)₂ |
| phenyl | 4-trifluoromethylphenyl | —C(O)NCH(CH₃)₂ |
| phenyl | 4-cyclopropylphenyl | —C(O)NCH(CH₃)₂ |
| phenyl | 4-diethylaminophenyl | —C(O)NCH(CH₃)₂ |
| phenyl | 4-difluoromethoxyphenyl | —C(O)NCH(CH₃)₂ |
| phenyl | 4-tert-butylphenyl | —C(O)OCH₃ |
| phenyl | 4-trifluoromethylphenyl | —C(O)OCH₃ |
| phenyl | 4-cyclopropylphenyl | —C(O)OCH₃ |
| phenyl | 4-diethylaminophenyl | —C(O)OCH₃ |
| phenyl | 4-difluoromethoxyphenyl | —C(O)OCH₃ |
| phenyl | 4-tert-butylphenyl | —C(O)OCH₂CH₃ |
| phenyl | 4-trifluoromethylphenyl | —C(O)OCH₂CH₃ |
| phenyl | 4-cyclopropylphenyl | —C(O)OCH₂CH₃ |
| phenyl | 4-diethylaminophenyl | —C(O)OCH₂CH₃ |
| phenyl | 4-difluoromethoxyphenyl | —C(O)OCH₂CH₃ |
| phenyl | 4-tert-butylphenyl | —C(O)OCH(CH₃)₂ |
| phenyl | 4-trifluoromethylphenyl | —C(O)OCH(CH₃)₂ |
| phenyl | 4-cyclopropylphenyl | —C(O)OCH(CH₃)₂ |
| phenyl | 4-diethylaminophenyl | —C(O)OCH(CH₃)₂ |
| phenyl | 4-difluoromethoxyphenyl | —C(O)OCH(CH₃)₂ |
| phenyl | 4-tert-butylphenyl | —C(O)OC(CH₃)₃ |
| phenyl | 4-trifluoromethylphenyl | —C(O)OC(CH₃)₃ |
| phenyl | 4-cyclopropylphenyl | —C(O)OC(CH₃)₃ |
| phenyl | 4-diethylaminophenyl | —C(O)OC(CH₃)₃ |
| phenyl | 4-difluoromethoxyphenyl | —C(O)OC(CH₃)₃ |
| phenyl | 4-tert-butylphenyl | —C(O)NHOH |
| phenyl | 4-trifluoromethylphenyl | —C(O)NHOH |
| phenyl | 4-cyclopropylphenyl | —C(O)NHOH |
| phenyl | 4-diethylaminophenyl | —C(O)NHOH |
| phenyl | 4-difluoromethoxyphenyl | —C(O)NHOH |
| phenyl | 4-tert-butylphenyl | —C(O)NHOCH₃ |
| phenyl | 4-trifluoromethylphenyl | —C(O)NHOCH₃ |
| phenyl | 4-cyclopropylphenyl | —C(O)NHOCH₃ |
| phenyl | 4-diethylaminophenyl | —C(O)NHOCH₃ |
| phenyl | 4-difluoromethoxyphenyl | —C(O)NHOCH₃ |
| phenyl | 4-tert-butylphenyl | —C(O)N(CH₃)OCH₃ |
| phenyl | 4-trifluoromethylphenyl | —C(O)N(CH₃)OCH₃ |
| phenyl | 4-cyclopropylphenyl | —C(O)N(CH₃)OCH₃ |
| phenyl | 4-diethylaminophenyl | —C(O)N(CH₃)OCH₃ |
| phenyl | 4-difluoromethoxyphenyl | —C(O)N(CH₃)OCH₃ |
| phenyl | 4-tert-butylphenyl | —C(NCN)NH₂ |
| phenyl | 4-trifluoromethylphenyl | —C(NCN)NH₂ |
| phenyl | 4-cyclopropylphenyl | —C(NCN)NH₂ |
| phenyl | 4-diethylaminophenyl | —C(NCN)NH₂ |
| phenyl | 4-difluoromethoxyphenyl | —C(NCN)NH₂ |
| phenyl | 4-tert-butylphenyl | —C(O)aziridin-1-yl |
| phenyl | 4-trifluoromethylphenyl | —C(O)aziridin-1-yl |
| phenyl | 4-cyclopropylphenyl | —C(O)aziridin-1-yl |
| phenyl | 4-diethylaminophenyl | —C(O)aziridin-1-yl |
| phenyl | 4-difluoromethoxyphenyl | —C(O)aziridin-1-yl |
| phenyl | 4-tert-butylphenyl | —C(O)azetidin-1-yl |
| phenyl | 4-trifluoromethylphenyl | —C(O)azetidin-1-yl |
| phenyl | 4-cyclopropylphenyl | —C(O)azetidin-1-yl |
| phenyl | 4-diethylaminophenyl | —C(O)azetidin-1-yl |
| phenyl | 4-difluoromethoxyphenyl | —C(O)azetidin-1-yl |
| phenyl | 4-tert-butylphenyl | —C(O)pyrrolidin-1-yl |
| phenyl | 4-trifluoromethylphenyl | —C(O)pyrrolidin-1-yl |
| phenyl | 4-cyclopropylphenyl | —C(O)pyrrolidin-1-yl |
| phenyl | 4-diethylaminophenyl | —C(O)pyrrolidin-1-yl |
| phenyl | 4-difluoromethoxyphenyl | —C(O)pyrrolidin-1-yl |
| phenyl | 4-tert-butylphenyl | —C(O)piperidin-1-yl |
| phenyl | 4-trifluoromethylphenyl | —C(O)piperidin-1-yl |
| phenyl | 4-cyclopropylphenyl | —C(O)piperidin-1-yl |
| phenyl | 4-diethylaminophenyl | —C(O)piperidin-1-yl |
| phenyl | 4-difluoromethoxyphenyl | —C(O)piperidin-1-yl |
| phenyl | 4-tert-butylphenyl | —C(O)piperazin-1-yl |
| phenyl | 4-trifluoromethylphenyl | —C(O)piperazin-1-yl |
| phenyl | 4-cyclopropylphenyl | —C(O)piperazin-1-yl |
| phenyl | 4-diethylaminophenyl | —C(O)piperazin-1-yl |
| phenyl | 4-difluoromethoxyphenyl | —C(O)piperazin-1-yl |
| phenyl | 4-tert-butylphenyl | —C(O)morpholin-4-yl |
| phenyl | 4-trifluoromethylphenyl | —C(O)morpholin-4-yl |
| phenyl | 4-cyclopropylphenyl | —C(O)morpholin-4-yl |
| phenyl | 4-diethylaminophenyl | —C(O)morpholin-4-yl |
| phenyl | 4-difluoromethoxyphenyl | —C(O)morpholin-4-yl |
| phenyl | 4-tert-butylphenyl | —C(O)imidazolin-1-yl |
| phenyl | 4-trifluoromethylphenyl | —C(O)imidazolin-1-yl |
| phenyl | 4-cyclopropylphenyl | —C(O)imidazolin-1-yl |
| phenyl | 4-diethylaminophenyl | —C(O)imidazolin-1-yl |
| phenyl | 4-difluoromethoxyphenyl | —C(O)imidazolin-1-yl |
| phenyl | 4-tert-butylphenyl | —C(O)isoxazolin-5-yl |
| phenyl | 4-trifluoromethylphenyl | —C(O)isoxazolin-5-yl |
| phenyl | 4-cyclopropylphenyl | —C(O)isoxazolin-5-yl |
| phenyl | 4-diethylaminophenyl | —C(O)isoxazolin-5-yl |
| phenyl | 4-difluoromethoxyphenyl | —C(O)isoxazolin-5-yl |
| phenyl | 4-tert-butylphenyl | —SO₂CH₃ |
| phenyl | 4-trifluoromethylphenyl | —SO₂CH₃ |
| phenyl | 4-cyclopropylphenyl | —SO₂CH₃ |
| phenyl | 4-diethylaminophenyl | —SO₂CH₃ |
| phenyl | 4-difluoromethoxyphenyl | —SO₂CH₃ |
| phenyl | 4-tert-butylphenyl | —SO₂CH₂CH₃ |
| phenyl | 4-trifluoromethylphenyl | —SO₂CH₂CH₃ |
| phenyl | 4-cyclopropylphenyl | —SO₂CH₂CH₃ |
| phenyl | 4-diethylaminophenyl | —SO₂CH₂CH₃ |
| phenyl | 4-difluoromethoxyphenyl | —SO₂CH₂CH₃ |
| phenyl | 4-tert-butylphenyl | —SO₂CH(CH₃)₂ |
| phenyl | 4-trifluoromethylphenyl | —SO₂CH(CH₃)₂ |
| phenyl | 4-cyclopropylphenyl | —SO₂CH(CH₃)₂ |
| phenyl | 4-diethylaminophenyl | —SO₂CH(CH₃)₂ |
| phenyl | 4-difluoromethoxyphenyl | —SO₂CH(CH₃)₂ |
| phenyl | 4-tert-butylphenyl | —SO₂C₆H₅ |
| phenyl | 4-trifluoromethylphenyl | —SO₂C₆H₅ |
| phenyl | 4-cyclopropylphenyl | —SO₂C₆H₅ |
| phenyl | 4-diethylaminophenyl | —SO₂C₆H₅ |
| phenyl | 4-difluoromethoxyphenyl | —SO₂C₆H₅ |
| 4-methoxyphenyl | 4-tert-butylphenyl | —H |
| 4-methoxyphenyl | 4-trifluoromethylphenyl | —H |
| 4-methoxyphenyl | 4-cyclopropylphenyl | —H |
| 4-methoxyphenyl | 4-diethylaminophenyl | —H |
| 4-methoxyphenyl | 4-difluoromethoxyphenyl | —H |
| 4-methoxyphenyl | 4-tert-butylphenyl | —C(O)CH₃ |
| 4-methoxyphenyl | 4-trifluoromethylphenyl | —C(O)CH₃ |
| 4-methoxyphenyl | 4-cyclopropylphenyl | —C(O)CH₃ |
| 4-methoxyphenyl | 4-diethylaminophenyl | —C(O)CH₃ |
| 4-methoxyphenyl | 4-difluoromethoxyphenyl | —C(O)CH₃ |
| 4-methoxyphenyl | 4-tert-butylphenyl | —C(O)cyclopropyl |
| 4-methoxyphenyl | 4-trifluoromethylphenyl | —C(O)cyclopropyl |
| 4-methoxyphenyl | 4-cyclopropylphenyl | —C(O)cyclopropyl |
| 4-methoxyphenyl | 4-diethylaminophenyl | —C(O)cyclopropyl |
| 4-methoxyphenyl | 4-difluoromethoxyphenyl | —C(O)cyclopropyl |
| 4-methoxyphenyl | 4-tert-butylphenyl | —C(O)NHCH₃ |
| 4-methoxyphenyl | 4-trifluoromethylphenyl | —C(O)NHCH₃ |
| 4-methoxyphenyl | 4-cyclopropylphenyl | —C(O)NHCH₃ |
| 4-methoxyphenyl | 4-diethylaminophenyl | —C(O)NHCH₃ |
| 4-methoxyphenyl | 4-difluoromethoxyphenyl | —C(O)NHCH₃ |

Compounds of the present invention include 4-oxo-1,3,8-triaza-spiro[4.5]-decanes having the formula (XIX) or a pharmaceutically acceptable salt form thereof:

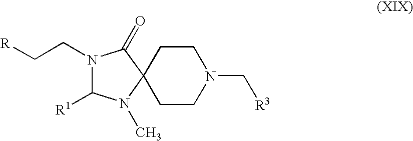

wherein non-limiting examples of R, R¹ and R³ are defined herein below in Table XI.

TABLE XI

| R | R¹ | R³ |
|---|---|---|
| 4-methoxyphenyl | 4-tert-butylphenyl | —C(O)CH₃ |
| 4-methoxyphenyl | 4-trifluoromethylphenyl | —C(O)CH₃ |
| 4-methoxyphenyl | 4-cyclopropylphenyl | —C(O)CH₃ |
| 4-methoxyphenyl | 4-diethylaminophenyl | —C(O)CH₃ |
| 4-methoxyphenyl | 4-difluoromethoxyphenyl | —C(O)CH₃ |
| 4-methoxyphenyl | 4-tert-butylphenyl | —C(O)C₃H₅ |
| 4-methoxyphenyl | 4-trifluoromethylphenyl | —C(O)C₃H₅ |
| 4-methoxyphenyl | 4-cyclopropylphenyl | —C(O)C₃H₅ |
| 4-methoxyphenyl | 4-diethylaminophenyl | —C(O)C₃H₅ |
| 4-methoxyphenyl | 4-difluoromethoxyphenyl | —C(O)C₃H₅ |
| 4-methoxyphenyl | 4-tert-butylphenyl | —C(O)NH₂ |
| 4-methoxyphenyl | 4-trifluoromethylphenyl | —C(O)NH₂ |
| 4-methoxyphenyl | 4-cyclopropylphenyl | —C(O)NH₂ |
| 4-methoxyphenyl | 4-diethylaminophenyl | —C(O)NH₂ |
| 4-methoxyphenyl | 4-difluoromethoxyphenyl | —C(O)NH₂ |
| 4-methoxyphenyl | 4-tert-butylphenyl | —C(O)NHCH₃ |
| 4-methoxyphenyl | 4-trifluoromethylphenyl | —C(O)NHCH₃ |
| 4-methoxyphenyl | 4-cyclopropylphenyl | —C(O)NHCH₃ |
| 4-methoxyphenyl | 4-diethylaminophenyl | —C(O)NHCH₃ |
| 4-methoxyphenyl | 4-difluoromethoxyphenyl | —C(O)NHCH₃ |
| 4-methoxyphenyl | 4-tert-butylphenyl | —C(O)NCH(CH₃)₂ |
| 4-methoxyphenyl | 4-trifluoromethylphenyl | —C(O)NCH(CH₃)₂ |
| 4-methoxyphenyl | 4-cyclopropylphenyl | —C(O)NCH(CH₃)₂ |
| 4-methoxyphenyl | 4-diethylaminophenyl | —C(O)NCH(CH₃)₂ |
| 4-methoxyphenyl | 4-difluoromethoxyphenyl | —C(O)NCH(CH₃)₂ |
| 4-methoxyphenyl | 4-tert-butylphenyl | —C(O)aziridin-1-yl |
| 4-methoxyphenyl | 4-trifluoromethylphenyl | —C(O)aziridin-1-yl |
| 4-methoxyphenyl | 4-cyclopropylphenyl | —C(O)aziridin-1-yl |
| 4-methoxyphenyl | 4-diethylaminophenyl | —C(O)aziridin-1-yl |
| 4-methoxyphenyl | 4-difluoromethoxyphenyl | —C(O)aziridin-1-yl |
| 4-methoxyphenyl | 4-tert-butylphenyl | —C(O)azetidin-1-yl |
| 4-methoxyphenyl | 4-trifluoromethylphenyl | —C(O)azetidin-1-yl |
| 4-methoxyphenyl | 4-cyclopropylphenyl | —C(O)azetidin-1-yl |
| 4-methoxyphenyl | 4-diethylaminophenyl | —C(O)azetidin-1-yl |
| 4-methoxyphenyl | 4-difluoromethoxyphenyl | —C(O)azetidin-1-yl |
| 4-methoxyphenyl | 4-tert-butylphenyl | —C(O)pyrrolidin-1-yl |
| 4-methoxyphenyl | 4-trifluoromethylphenyl | —C(O)pyrrolidin-1-yl |
| 4-methoxyphenyl | 4-cyclopropylphenyl | —C(O)pyrrolidin-1-yl |
| 4-methoxyphenyl | 4-diethylaminophenyl | —C(O)pyrrolidin-1-yl |
| 4-methoxyphenyl | 4-difluoromethoxyphenyl | —C(O)pyrrolidin-1-yl |
| 4-methoxyphenyl | 4-tert-butylphenyl | —C(O)piperidin-1-yl |
| 4-methoxyphenyl | 4-trifluoromethylphenyl | —C(O)piperidin-1-yl |
| 4-methoxyphenyl | 4-cyclopropylphenyl | —C(O)piperidin-1-yl |
| 4-methoxyphenyl | 4-diethylaminophenyl | —C(O)piperidin-1-yl |
| 4-methoxyphenyl | 4-difluoromethoxyphenyl | —C(O)piperidin-1-yl |
| 4-methoxyphenyl | 4-tert-butylphenyl | —C(O)piperazin-1-yl |
| 4-methoxyphenyl | 4-trifluoromethylphenyl | —C(O)piperazin-1-yl |
| 4-methoxyphenyl | 4-cyclopropylphenyl | —C(O)piperazin-1-yl |
| 4-methoxyphenyl | 4-diethylaminophenyl | —C(O)piperazin-1-yl |
| 4-methoxyphenyl | 4-difluoromethoxyphenyl | —C(O)piperazin-1-yl |
| 4-methoxyphenyl | 4-tert-butylphenyl | —C(O)morpholin-4-yl |
| 4-methoxyphenyl | 4-trifluoromethylphenyl | —C(O)morpholin-4-yl |
| 4-methoxyphenyl | 4-cyclopropylphenyl | —C(O)morpholin-4-yl |
| 4-methoxyphenyl | 4-diethylaminophenyl | —C(O)morpholin-4-yl |
| 4-methoxyphenyl | 4-difluoromethoxyphenyl | —C(O)morpholin-4-yl |
| 4-methoxyphenyl | 4-tert-butylphenyl | —C(O)imidazolin-1-yl |
| 4-methoxyphenyl | 4-trifluoromethylphenyl | —C(O)imidazolin-1-yl |
| 4-methoxyphenyl | 4-cyclopropylphenyl | —C(O)imidazolin-1-yl |
| 4-methoxyphenyl | 4-diethylaminophenyl | —C(O)imidazolin-1-yl |
| 4-methoxyphenyl | 4-difluoromethoxyphenyl | —C(O)imidazolin-1-yl |

Compounds of the present invention include 4-oxo-1,3,8-triaza-spiro[4.5]-decanes having the formula (XX) or a pharmaceutically acceptable salt form thereof:

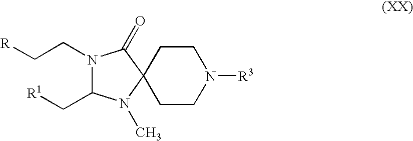

wherein non-limiting examples of R, R¹ and R³ are defined herein below in Table XII.

TABLE XII

| R | R¹ | R³ |
|---|---|---|
| phenyl | 4-tert-butylphenyl | —H |
| phenyl | 4-trifluoromethylphenyl | —H |
| phenyl | 4-cyclopropylphenyl | —H |
| phenyl | 4-diethylaminophenyl | —H |
| phenyl | 4-difluoromethoxyphenyl | —H |
| phenyl | 4-tert-butylphenyl | —C(O)CH₃ |
| phenyl | 4-trifluoromethylphenyl | —C(O)CH₃ |
| phenyl | 4-cyclopropylphenyl | —C(O)CH₃ |
| phenyl | 4-diethylaminophenyl | —C(O)CH₃ |
| phenyl | 4-difluoromethoxyphenyl | —C(O)CH₃ |
| phenyl | 4-tert-butylphenyl | —C(O)cyclopropyl |
| phenyl | 4-trifluoromethylphenyl | —C(O)cyclopropyl |
| phenyl | 4-cyclopropylphenyl | —C(O)cyclopropyl |
| phenyl | 4-diethylaminophenyl | —C(O)cyclopropyl |
| phenyl | 4-difluoromethoxyphenyl | —C(O)cyclopropyl |
| phenyl | 4-tert-butylphenyl | —C(O)NH₂ |
| phenyl | 4-trifluoromethylphenyl | —C(O)NH₂ |
| phenyl | 4-cyclopropylphenyl | —C(O)NH₂ |
| phenyl | 4-diethylaminophenyl | —C(O)NH₂ |
| phenyl | 4-difluoromethoxyphenyl | —C(O)NH₂ |
| phenyl | 4-tert-butylphenyl | —C(O)NHCH₃ |
| phenyl | 4-trifluoromethylphenyl | —C(O)NHCH₃ |
| phenyl | 4-cyclopropylphenyl | —C(O)NHCH₃ |
| phenyl | 4-diethylaminophenyl | —C(O)NHCH₃ |
| phenyl | 4-difluoromethoxyphenyl | —C(O)NHCH₃ |
| phenyl | 4-tert-butylphenyl | —C(O)N(CH₃)₂ |
| phenyl | 4-trifluoromethylphenyl | —C(O)N(CH₃)₂ |
| phenyl | 4-cyclopropylphenyl | —C(O)N(CH₃)₂ |
| phenyl | 4-diethylaminophenyl | —C(O)N(CH₃)₂ |
| phenyl | 4-difluoromethoxyphenyl | —C(O)N(CH₃)₂ |
| phenyl | 4-tert-butylphenyl | C(O)NH[CH(CH₃)₂] |
| phenyl | 4-trifluoromethylphenyl | C(O)NH[CH(CH₃)₂] |
| phenyl | 4-cyclopropylphenyl | C(O)NH[CH(CH₃)₂] |
| phenyl | 4-diethylaminophenyl | C(O)NH[CH(CH₃)₂] |
| phenyl | 4-difluoromethoxyphenyl | C(O)NH[CH(CH₃)₂] |
| phenyl | 4-tert-butylphenyl | —C(O)OCH₃ |
| phenyl | 4-trifluoromethylphenyl | —C(O)OCH₃ |
| phenyl | 4-cyclopropylphenyl | —C(O)OCH₃ |
| phenyl | 4-diethylaminophenyl | —C(O)OCH₃ |
| phenyl | 4-difluoromethoxyphenyl | —C(O)OCH₃ |
| phenyl | 4-tert-butylphenyl | —C(O)OCH₂CH₃ |
| phenyl | 4-trifluoromethylphenyl | —C(O)OCH₂CH₃ |
| phenyl | 4-cyclopropylphenyl | —C(O)OCH₂CH₃ |
| phenyl | 4-diethylaminophenyl | —C(O)OCH₂CH₃ |
| phenyl | 4-difluoromethoxyphenyl | —C(O)OCH₂CH₃ |
| phenyl | 4-tert-butylphenyl | —C(O)OCH(CH₃)₂ |
| phenyl | 4-trifluoromethylphenyl | —C(O)OCH(CH₃)₂ |
| phenyl | 4-cyclopropylphenyl | —C(O)OCH(CH₃)₂ |
| phenyl | 4-diethylaminophenyl | —C(O)OCH(CH₃)₂ |
| phenyl | 4-difluoromethoxyphenyl | —C(O)OCH(CH₃)₂ |
| phenyl | 4-tert-butylphenyl | —C(O)OC(CH₃)₃ |
| phenyl | 4-trifluoromethylphenyl | —C(O)OC(CH₃)₃ |
| phenyl | 4-cyclopropylphenyl | —C(O)OC(CH₃)₃ |
| phenyl | 4-diethylaminophenyl | —C(O)OC(CH₃)₃ |
| phenyl | 4-difluoromethoxyphenyl | —C(O)OC(CH₃)₃ |
| phenyl | 4-tert-butylphenyl | —C(O)NHOH |
| phenyl | 4-trifluoromethylphenyl | —C(O)NHOH |
| phenyl | 4-cyclopropylphenyl | —C(O)NHOH |
| phenyl | 4-diethylaminophenyl | —C(O)NHOH |
| phenyl | 4-difluoromethoxyphenyl | —C(O)NHOH |
| phenyl | 4-tert-butylphenyl | —C(O)NHOCH₃ |
| phenyl | 4-trifluoromethylphenyl | —C(O)NHOCH₃ |
| phenyl | 4-cyclopropylphenyl | —C(O)NHOCH₃ |
| phenyl | 4-diethylaminophenyl | —C(O)NHOCH₃ |
| phenyl | 4-difluoromethoxyphenyl | —C(O)NHOCH₃ |
| phenyl | 4-tert-butylphenyl | —C(O)N(CH₃)OCH₃ |
| phenyl | 4-trifluoromethylphenyl | —C(O)N(CH₃)OCH₃ |
| phenyl | 4-cyclopropylphenyl | —C(O)N(CH₃)OCH₃ |
| phenyl | 4-diethylaminophenyl | —C(O)N(CH₃)OCH₃ |
| phenyl | 4-difluoromethoxyphenyl | —C(O)N(CH₃)OCH₃ |
| phenyl | 4-tert-butylphenyl | —C(NCN)NH₂ |
| phenyl | 4-trifluoromethylphenyl | —C(NCN)NH₂ |
| phenyl | 4-cyclopropylphenyl | —C(NCN)NH₂ |
| phenyl | 4-diethylaminophenyl | —C(NCN)NH₂ |
| phenyl | 4-difluoromethoxyphenyl | —C(NCN)NH₂ |
| phenyl | 4-tert-butylphenyl | —C(O)aziridin-1-yl |
| phenyl | 4-trifluoromethylphenyl | —C(O)aziridin-1-yl |
| phenyl | 4-cyclopropylphenyl | —C(O)aziridin-1-yl |
| phenyl | 4-diethylaminophenyl | —C(O)aziridin-1-yl |
| phenyl | 4-difluoromethoxyphenyl | —C(O)aziridin-1-yl |
| phenyl | 4-tert-butylphenyl | —C(O)azetidin-1-yl |
| phenyl | 4-trifluoromethylphenyl | —C(O)azetidin-1-yl |
| phenyl | 4-cyclopropylphenyl | —C(O)azetidin-1-yl |
| phenyl | 4-diethylaminophenyl | —C(O)azetidin-1-yl |
| phenyl | 4-difluoromethoxyphenyl | —C(O)azetidin-1-yl |
| phenyl | 4-tert-butylphenyl | —C(O)pyrrolidin-1-yl |
| phenyl | 4-trifluoromethylphenyl | —C(O)pyrrolidin-1-yl |
| phenyl | 4-cyclopropylphenyl | —C(O)pyrrolidin-1-yl |
| phenyl | 4-diethylaminophenyl | —C(O)pyrrolidin-1-yl |
| phenyl | 4-difluoromethoxyphenyl | —C(O)pyrrolidin-1-yl |
| phenyl | 4-tert-butylphenyl | —C(O)piperidin-1-yl |
| phenyl | 4-trifluoromethylphenyl | —C(O)piperidin-1-yl |
| phenyl | 4-cyclopropylphenyl | —C(O)piperidin-1-yl |
| phenyl | 4-diethylaminophenyl | —C(O)piperidin-1-yl |
| phenyl | 4-difluoromethoxyphenyl | —C(O)piperidin-1-yl |
| phenyl | 4-tert-butylphenyl | —C(O)piperazin-1-yl |
| phenyl | 4-trifluoromethylphenyl | —C(O)piperazin-1-yl |
| phenyl | 4-cyclopropylphenyl | —C(O)piperazin-1-yl |
| phenyl | 4-diethylaminophenyl | —C(O)piperazin-1-yl |
| phenyl | 4-difluoromethoxyphenyl | —C(O)piperazin-1-yl |
| phenyl | 4-tert-butylphenyl | —C(O)morpholin-4-yl |
| phenyl | 4-trifluoromethylphenyl | —C(O)morpholin-4-yl |
| phenyl | 4-cyclopropylphenyl | —C(O)morpholin-4-yl |
| phenyl | 4-diethylaminophenyl | —C(O)morpholin-4-yl |
| phenyl | 4-difluoromethoxyphenyl | —C(O)morpholin-4-yl |
| phenyl | 4-tert-butylphenyl | —C(O)imidazolin-1-yl |
| phenyl | 4-trifluoromethylphenyl | —C(O)imidazolin-1-yl |
| phenyl | 4-cyclopropylphenyl | —C(O)imidazolin-1-yl |
| phenyl | 4-diethylaminophenyl | —C(O)imidazolin-1-yl |
| phenyl | 4-difluoromethoxyphenyl | —C(O)imidazolin-1-yl |
| phenyl | 4-tert-butylphenyl | —C(O)isoxazolin-5-yl |
| phenyl | 4-trifluoromethylphenyl | —C(O)isoxazolin-5-yl |
| phenyl | 4-cyclopropylphenyl | —C(O)isoxazolin-5-yl |
| phenyl | 4-diethylaminophenyl | —C(O)isoxazolin-5-yl |
| phenyl | 4-difluoromethoxyphenyl | —C(O)isoxazolin-5-yl |
| phenyl | 4-tert-butylphenyl | —SO₂CH₃ |
| phenyl | 4-trifluoromethylphenyl | —SO₂CH₃ |
| phenyl | 4-cyclopropylphenyl | —SO₂CH₃ |
| phenyl | 4-diethylaminophenyl | —SO₂CH₃ |
| phenyl | 4-difluoromethoxyphenyl | —SO₂CH₃ |
| phenyl | 4-tert-butylphenyl | —SO₂CH₂CH₃ |
| phenyl | 4-trifluoromethylphenyl | —SO₂CH₂CH₃ |
| phenyl | 4-cyclopropylphenyl | —SO₂CH₂CH₃ |
| phenyl | 4-diethylaminophenyl | —SO₂CH₂CH₃ |
| phenyl | 4-difluoromethoxyphenyl | —SO₂CH₂CH₃ |
| phenyl | 4-tert-butylphenyl | —SO₂CH(CH₃)₂ |
| phenyl | 4-trifluoromethylphenyl | —SO₂CH(CH₃)₂ |
| phenyl | 4-cyclopropylphenyl | —SO₂CH(CH₃)₂ |
| phenyl | 4-diethylaminophenyl | —SO₂CH(CH₃)₂ |
| phenyl | 4-difluoromethoxyphenyl | —SO₂CH(CH₃)₂ |
| phenyl | 4-tert-butylphenyl | —SO₂C₆H₅ |
| phenyl | 4-trifluoromethylphenyl | —SO₂C₆H₅ |
| phenyl | 4-cyclopropylphenyl | —SO₂C₆H₅ |
| phenyl | 4-diethylaminophenyl | —SO₂C₆H₅ |
| phenyl | 4-difluoromethoxyphenyl | —SO₂C₆H₅ |

Compounds of the present invention include 4-oxo-1,3,8-triaza-spiro[4.5]decanes having the formula (XXI) or a pharmaceutically acceptable salt form thereof:

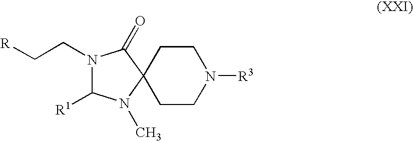

wherein R³ is —C(O)R⁴ and R⁴ is a substituted C₁-C₆ linear or branched, or C₃-C₆ substituted cycloalkyl.

Compounds of the present invention include 4-oxo-1,3,8-triaza-spiro[4.5]decanes having the formula (XXI) or a pharmaceutically acceptable salt form thereof: 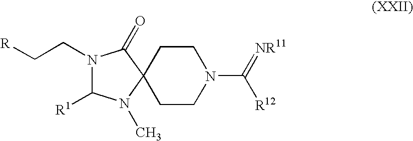
wherein R, R¹, R¹¹, and R¹² are as defined herein.

The Examples provided below provide representative methods for preparing exemplary compounds of the present invention. The skilled practitioner will know how to substitute the appropriate reagents, starting materials and purification methods known to those skilled in the art, in order to prepare the compounds of the present invention.

### EXAMPLE 1

Example 1 provides methods for preparing representative compounds of formula (IX). The skilled practitioner will know how to substitute the appropriate reagents, starting materials and purification methods known to those skilled in the art, in order to prepare additional compounds of the present invention.

### Compound 1: 2-(4-Cyclopropylphenyl)-3-[2-(4-methoxyphenyl)ethyl]-1-methyl-1,3,8-triaza-spiro[4.5]decan-4-one

Preparation of tert-butyl-4-[(4-methoxyphenethyl)carbamoyl]-4-{[(9H-fluoren-9-yl)methoxy]carbonyl}piperidine-1-carboxylate: To a solution of 1-N-Boc-4-N-Fmoc-amino-4-carboxypiperidine (4.66 g, 10 mmol) in DMF (30 mL) is added benzotriazole-2-yl-(oxy-tris-pyrrolidino)-phosphonium hexafluorophosphate (PyBOP) (5.2 g, 10 mmol). After stirring at room temperature for 10 minutes, 4-methoxyphenethyl amine (1.51 g, 10 mmol) is added, and the solution is stirred for a further 5 minutes. Diisopropyl amine (6 drops) is added, and the solution is stirred for 3 hours at room temperature. The reaction mixture is diluted with EtOAc (250 mL) and is washed with aqueous KHSO₄ (10%). The phases are separated, and the aqueous phase is extracted with EtOAc. The combined organic phase is washed with brine and dried over Na₂SO₄. The solvent is removed in vacuo, and the resulting residue is purified over silica to provide 4.93 g (80% yield) of the desired product.

Preparation of tert-butyl-4-[(4-methoxyphenethyl)carbamoyl]-4-aminopiperidine-1-carboxylate: To a solution of the tert-butyl-4-[(4-methoxyphenethyl)carbamoyl]-4-{[(9H-fluoren-9-yl)methoxy]carbonyl}piperidine-1-carboxylate, 1, (4.93 g, 8.0 mmol) in DMF (30 mL) is added piperidine (2 mL). The solution is stirred at room temperature for 3 hours, and the precipitate which forms is filtered off and washed with MeOH. The filtrate is allowed to stand until a precipitate has re-formed. This procedure of collecting the precipitate is repeated until no more precipitate forms from the filtrate. The solvent is removed in vacuo to afford 3.2 g of the desired product as a viscous crude yellow oil which is used without further purification.

Preparation of 2-(4-cyclopropylphenyl)-3-[2-(4-methoxyphenyl)ethyl]-4-oxo-1,3,8-triaza-spiro[4.5]decane-8-carboxylic acid tert-butyl ester: To the solution of tert-butyl 4-[(4-methoxyphenethyl)carbamoyl]-4-aminopiperidine-1-carboxylate (900 mg, 2.38 mmol in 4 mL of methanol) and K₂CO₃ (276 mg, 2.0 mmol) in a 2.0-5.0 mL Emry's process vial equipped with a stir bar is added 4-cyclopropylbenzaldehyde (350 mg, 2.4 mmol) via pipette. The reaction mixture is then capped, stirred 20 seconds and heated in a Biotage Initiator 60 microwave for 20 minutes at 120° C. The reaction is then cooled to room temperature, diluted with ethyl acetate (100 mL), washed with water (2×50 mL), dried over Na₂SO₄ and purified over silica to afford 507 mg (50% yield) of the desired product. ¹H-NMR (300 MHz, CDCl₃) δ 7.40 (m, 4H), 7.05 (d, 2H, J=8.8 Hz), 6.76 (d, 2H, J=8.7 Hz), 5.04 (s, 1H), 4.10 (m, 1H), 3.92 (m, 2H), 3.81 (s, 3H), 3.20 (m, 1H), 3.05 (m, 1H), 2.80 (m, 2H), 2.56 (m, 1H), 2.12 (m, 1H), 1.80 (m, 1H), 1.58 (m, 3H), 1.40 (s, 9H), 1.25 (m, 1H), 1.00 (m, 2H), 0.7 (m, 2H); ¹³C-NMR (75 MHz, CDCl₃) δ 177.0; 158.7, 155.0, 146.3, 135.5, 130.6, 130.1, 127.5, 126.7, 114.3, 79.9, 74.7, 60.4, 55.6, 41.8, 39.7, 39.4, 34.5, 32.6, 31.9, 28.8, 15.6, 10.0; MS MH⁺=506.2; elemental analysis: theory C₃₀H₃₉N₃O₄+0.1 CF₃COOH C, 70.15; H, 7.62; N, 8.13. found C, 70.32, H, 7.37, N, 8.11.

Preparation of 2-(4-cyclopropylphenyl)-3-[2-(4-methoxyphenyl)ethyl]-1-methyl-4-oxo-1,3,8-triaza-spiro[4.5]decane-8-carboxylic acid tert-butyl ester: To the solution of the 2-(4-cyclopropylphenyl)-3-[2-(4-methoxyphenyl)ethyl]-4-oxo-1,3,8-triaza-spiro[4.5]decane-8-carboxylic acid tert-butyl ester, 3, (858 mg, 1.7 mmol in 4 mL of DMF) and CsCO₃ (648 mg, 2.0 mmol) in a 2.0-5.0 mL Emry's process vial equipped with a stir bar is added MeI (479 mg, 3.4 mmol) via pipette. The reaction mixture is then capped, stirred 30 seconds and heated in a Biotage Initiator 60 microwave for 25 minutes at 90° C. The reaction is then cooled to room temperature and diluted with EtOAc (100 mL) and washed with water (2×50 mL). The remaining aqueous layer is then extracted with EtOAc (2×30 mL). The combined organic extracts are then dried over anhydrous Na₂SO₄ and evaporated to dryness. The crude residue is then purified over silica to afford 512 mg (58% yield) of the desired product. ¹H-NMR (300 MHz, CDCl₃) δ 7.23 (d, 2H, J=8.1 Hz), 7.10 (d, 2H, J=8.0 Hz), 7.05 (d, 2H, J=8.4 Hz), 6.84 (d, 2H, J=8.4 Hz), 4.52 (s, 1H), 4.10 (m, 1H), 3.90 (m, 2H), 3.80 (s, 3H), 3.20 (m, 1H), 3.05 (m, 1H), 2.76 (m, 2H), 2.50 (m, 1H), 2.03 (s, 3H), 1.93 (m, 1H), 1.58 (m, 3H), 1.40 (s, 9H), 1.15 (m, 1H), 1.01 (m, 2H), 0.7 (m, 2H); ¹³C-NMR (75 MHz, CDCl₃) δ 175.0; 158.7, 155.0, 146.1, 134.5, 130.4, 130.2, 128.9, 126.1, 114.1, 79.8, 79.7, 60.4, 55.6, 41.0, 40.6, 40.4, 32.9, 32.3, 30.4, 28.8, 15.6, 10.0; MS MH⁺=520.1; elemental analysis: theory C₃₁H₄₁N₃O₄ C, 71.65, H, 7.95, N, 8.09. found C, 71.98; H, 7.57; N, 7.83.

Preparation of 2-(4-cyclopropylphenyl)-3-[2-(4-methoxyphenyl)ethyl]-1-methyl-1,3,8-triaza-spiro[4.5]decan-4-one: To a solution of the 2-(4-cyclopropylphenyl)-3-[2-(4-methoxyphenyl)ethyl]-1-methyl-4-oxo-1,3,8-triaza-spiro[4.5]decane-8-carboxylic acid tert-butyl ester, 4, (10.26 g, 19.7 mmol) in CH₂Cl₂ (100 mL) is added trifluoroacetic acid (25 mL). After stirring at room temperature for 3 hours, the aqueous NaHCO₃ (saturated, 200 mL) is added slowly and resulting mixture is stirred for 30 minutes at room temperature. The resulting two layers are separated and the aqueous layer is extracted with CH₂Cl₂ (100 mL). The organic layers are combined and washed with aqueous NaHCO₃ and dried over NaSO₄. The solvent is removed in vacuo to afford 9.2 g (87% yield) of the desired product as a white solid. ¹H-NMR (300 MHz, CDCl₃) δ 7.23 (d, 2H, J=8.0 Hz), 7.11 (d, 2H, J=8.5 Hz), 7.07 (d, 2H, J=8.6 Hz), 6.84 (d, 2H, J=8.6 Hz), 4.54 (s, 1H), 4.07 (m, 1H), 3.89 (m, 2H), 3.79 (s, 3H), 3.17 (m, 3H), 2.79 (m, 2H), 2.53 (m, 1H), 2.08 (s, 3H), 1.93 (m, 4H), 1.22 (m, 1H), 1.04 (m, 2H), 0.77 (m, 2H); ¹³C-NMR (75 MHz, CDCl₃) δ 175.0; 158.7, 146.2, 134.3, 130.4, 130.3, 128.8, 126.2, 114.1, 79.8, 59.5, 55.6, 42.0, 41.5, 40.4, 32.9, 31.8, 30.3, 25.7, 15.6, 10.0, 9.9; MS MH⁺=420.5; elemental analysis: theory C₂₇H₃₄N₄O₃+0.9H₂O C, 61.20, H, 6.22; N, 9.91. found C, 60.98; H, 6.62; N, 10.00.

The following are further non-limiting examples of compounds of formula (IX).

### Compound 2: 2-(4-tert-Butylphenyl)-3-[2-(4-methoxyphenyl)ethyl]-1-methyl-1,3,8-triaza-spiro[4.5]decan-4-one

¹H-NMR (300 MHz, CDCl₃) δ 7.47 (d, 2H, J=8.7 Hz), 7.29 (d, 2H, J=8.7 Hz), 7.07 (d, 2H, J=8.9 Hz), 6.85 (d, 2H, J=8.7 Hz), 4.66 (s, 1H), 3.80 (s, 3H), 3.74 (m, 3H), 3.11 (m, 1H), 2.80 (s, 3H), 2.78 (m, 2H), 2.52 (m, 1H), 2.13 (s, 3H), 1.93 (m, 2H), 1.85 (m, 1H), 1.36 (s, 9H), 1.31 (m, 2H); ¹³C-NMR (75 MHz, CDCl₃) δ 175.0; 158.7, 155.4, 155.0, 134.5, 130.3, 129.9, 128.6, 126.2, 114.2, 79.9, 58.9, 55.6, 41.6, 41.2, 40.3, 35.1, 32.7, 31.7, 30.2, 29.4, 24.0; MS MH⁺=436.1; elemental analysis: theory C₂₇H₃₇N₃O₂+2.3 CF₃COOH C, 54.39; H, 5.68; N, 6.02. found C, 54.27; H, 5.67; N, 5.94.

### Compound 3: 2-(4-Difluoromethoxyphenyl)-3-[2-(4-methoxyphenyl)ethyl]-1-methyl-1,3,8-triaza-spiro[4.5]decan-4-one

¹H-NMR (300 MHz, CDCl₃) δ 7.35 (d, 2H, J=8.4 Hz), 7.18 (d, 2H, J=8.5 Hz), 7.07 (d, 2H, J=8.4 Hz), 6.85 (d, 2H, J=8.6 Hz), 6.57, 6.33 (s, s, 1H), 4.56 (s, 1H), 3.90 (m, 2H), 3.80 (s, 3H), 3.16 (m, 3H), 2.81 (m, 1H), 2.69 (m, 1H), 2.53 (m, 1H), 2.08 (s, 3H), 1.91 (m, 4H), 1.24 (m, 1H); ¹³C-NMR (75 MHz, CDCl₃) δ 175.0; 158.7, 152.4, 134.6, 130.4, 130.2, 120.0, 119.5, 116.0, 114.2, 112.6, 79.3, 59.6, 55.6, 42.0, 41.6, 40.5, 33.0, 32.0, 30.3, 26.0; MS MH⁺=446.4; elemental analysis: theory C₂₄H₂₉F₂N₃O₃+0.5 CF₃COOH C, 59.75; H, 5.92; N, 8.36. found C, 59.41; H, 5.92; N, 8.19.

### EXAMPLE 2

Example 2 provides methods for preparing representative compounds of formula (X). The skilled practitioner will know how to substitute the appropriate reagents, starting materials and purification methods known to those skilled in the art, in order to prepare additional compounds of the present invention.

### Compound 4: 8-Cyclopropylcarbonyl-2-(4-cyclopropylphenyl)-3-[2-(4-methoxyphenyl)ethyl]-1-methyl-1,3,8-triaza-spiro[4.5]decan-4-one

Preparation of 8-cyclopropylcarbonyl-2-(4-cyclopropylphenyl)-3-[2-(4-methoxyphenyl)ethyl]-1-methyl-1,3,8-triaza-spiro[4.5]decan-4-one: To a solution of 2-(4-cyclopropylphenyl)-3-[2-(4-methoxyphenyl)ethyl]-1-methyl-1,3,8-triaza-spiro[4.5]decan-4-one, 5, (238 mg contained 0.5 mmol TFA salt, 0.5 mmol) in CH₂Cl₂ (15 mL) is added triethylamine (200 mg, 2 mmol) and cyclopropanecarbonyl chloride (208 mg, 2 mmol). The solution is stirred for 5 hours at room temperature. Methylene chloride (100 mL) is added and the resulting mixture is washed with NaHCO₃ (saturated aqueous), H₂O, dried over Na₂SO₄ and purified over silica to afford 92.7 mg (35% yield) of the desired product. ¹H-NMR (300 MHz, CDCl₃) δ 7.24 (d, 2H, J=8.6 Hz), 7.12 (d, 2H, J=8.6 Hz), 7.07 (d, 2H, J=8.6 Hz), 6.85 (d, 2H, J=8.6 Hz), 4.65 (m, 1H), 4.59 (s, 1H), 4.24 (m, 2H), 3.91 (m, 1H), 3.80 (s, 3H), 3.20 (m, 1H), 2.78 (m, 2H), 2.69 (m, 1H), 2.06 (s, 3H), 1.97 (m, 2H), 1.81 (m, 3H), 1.22 (m, 1H), 1.06 (m, 4H), 0.82 (m, 4H); ¹³C-NMR (75 MHz, CDCl₃) δ 176.0; 173.0, 158.7, 146.4, 133.7, 130.4, 130.2, 128.9, 126.2, 114.2, 79.8, 60.6, 55.7, 40.6, 32.9, 30.4, 15.6, 11.4, 10.1, 10.0, 7.8; MS MH⁺=488.3; HRMS: theory C₃₀H₃₇N₃O₃ 488.2913; found 488.2922.

The following are further non-limiting examples of compounds of formula X of the present invention

### Compound 5: 8-Acetyl-2-(4-cyclopropylphenyl)-3-[2-(4-methoxyphenyl)ethyl]-1-methyl-1,3,8-triaza-spiro[4.5]decan-4-one

¹H NMR (CDCl₃) δ 7.21 (d, 2H, J=8.0 Hz), 7.09 (d, 2H, J=8.1 Hz), 6.84-6.80 (m, 2H), 4.61-4.41 (m, 2H), 4.26-4.07 (m, 1H), 3.97-3.45 (m, 6H), 3.07-2.87 (m, 1H), 2.84-2.60 (m, 2H), 2.58-2.41 (m, 1H), 2.11 (s, 3H), 2.03 (s, 3H), 1.98-1.78 (m, 2H), 1.75-1.48 (m, 2H), 1.32-1.11 (m, 1H), 1.08-0.96 (m, 2H), 0.81-0.67 (m, 2H); ESI-MS (m/z): (M+H⁺) 462.

### Compound 6: 8-Cyclopropylcarbonyl-2-(4-difluoromethoxyphenyl)-3-[2-(4-methoxyphenyl)-ethyl]-1-methyl-1,3,8-triazaspiro[4.5]decan-4-one

¹H-NMR (300 MHz, CDCl₃) δ 7.33 (d, 2H, J=8.4 Hz), 7.16 (d, 2H, J=8.4 Hz), 7.04 (d, 2H, J=8.4 Hz), 6.83 (d, 2H, J=8.4 Hz), 6.57 (t, 1H, J=81.6 Hz), 4.57 (s, 1H), 4.45 (m, 1H), 4.20 (m, 0.5H), 4.13 (m, 1H), 3.88 (m, 1H), 3.80 (s, 3H), 3.63 (m, 1H), 3.03 (m, 0.5H), 2.79 (m, 1H), 2.70 (m, 1H), 2.55 (m, 1H), 2.03 (s, 3H), 1.72 (m, 4H), 1.29 (m, 1H), 0.99 (m, 2H), 0.78 (m, 2H); MH⁺=514.2; elemental analysis: theory C₂₈H₃₃F₂N₃O₄ C, 65.48; H, 6.48; N, 8.18. found C, 65.83; H, 6.46; N, 8.09.

### EXAMPLE 3

Example 3 provides methods for preparing representative compounds of formula (XI). The skilled practitioner will know how to substitute the appropriate reagents, starting materials and purification methods known to those skilled in the art, in order to prepare additional compounds of the present invention.

### Compound 7: 2-(4-Cyclopropylphenyl)-3-[2-(4-methoxyphenyl)ethyl]-1-methyl-4-oxo-1,3,8-triaza-spiro[4.5]decane-8-carboxylic acid amide

Preparation of 2-(4-cyclopropylphenyl)-3-[2-(4-methoxyphenyl)ethyl]-1-methyl-4-oxo-1,3,8-triaza-spiro[4.5]decane-8-carboxylic acid amide: To a solution of the 2-(4-cyclopropylphenyl)-3-[2-(4-methoxyphenyl)ethyl]-1-methyl-1,3,8-triaza-spiro[4.5]decan-4-one, 5, (3.0 g, 7.1 mmol) in CH₂Cl₂ (100 mL) is added trimethylsilyl isocyanide (2.4 g, 21.4 mmol), TEA (0.84 mL, 7.3 mmol). After stirring at room temperature for 18 hours, the aqueous NaHCO₃ (saturate, 50 mL) is added and resulting mixture is stirred for 30 minutes at the room temperature. Two layers are separated and aqueous layer is extracted with CH₂Cl₂ (2×100 mL). The organic layers are combined and washed with aqueous NaHCO₃ and dried over NaSO₄. The solvent is removed in vacuo to a crude residue which is purified over silica to afford 2.48 g (85% yield) of the desired product. ¹H-NMR (300 MHz, CDCl₃) δ 7.20 (d, 2H, J=8.2 Hz), 7.10 (d, 2H, J=8.2 Hz), 7.03 (d, 2H, J=8.6 Hz), 6.84 (d, 2H, J=8.7 Hz), 4.60 (b, 2H), 4.54 (s, 1H), 3.99 (m, 1H), 3.89 (m, 3H), 3.80 (s, 3H), 3.23 (m, 1H), 2.76 (m, 2H), 2.51 (m, 1H), 2.04 (s, 3H), 1.93 (m, 1H), 1.76 (m, 3H), 1.21 (m, 1H), 1.03 (m, 2H), 0.74 (m, 2H); ¹³C-NMR (75 MHz, CDCl₃) δ 175.0; 158.7, 158.4, 146.1, 134.4, 130.5, 130.2, 128.9, 126.2, 114.1, 79.8, 60.3, 55.7, 41.5, 40.5, 40.4, 32.9, 32.6, 30.4, 26.3, 15.6, 10.0, 9.9; MS MH⁺=463.3; elemental analysis: theory C₂₇H₃₄N₄O₃ C, 70.10; H, 7.41; N, 12.11. found C, 70.07; H, 7.47; N, 12.09.

For exemplary compounds of formula (XI) wherein one of R⁵ or R⁶ are C₁-C₄ linear or branched alkyl, the procedure exemplified in Example 4 can be followed. The skilled practitioner will know how to substitute the appropriate reagents, starting materials and purification methods known to those skilled in the art, in order to prepare the compounds provided herein.

### EXAMPLE 4

### Compound 8: 2-(4-Cyclopropylphenyl)-3-[2-(4-methoxyphenyl)ethyl]-1-methyl-4-oxo-1,3,8-triaza-spiro[4.5]decane-8-carboxylic acid methyl amide

Preparation of 2-(4-cyclopropylphenyl)-3-[2-(4-methoxyphenyl)ethyl]-1-methyl-4-oxo-1,3,8-triaza-spiro[4.5]decane-8-carboxylic acid methyl amide: To a solution of 2-(4-cyclopropylphenyl)-3-[2-(4-methoxyphenyl)ethyl]-1-methyl-,3,8-triaza-spiro[4.5]decan-4-one, 5, (238 mg contained 0.5 mmol TFA salt, 0.5 mmol) in CH₂Cl₂ (10 mL) is added triethylamine (152 mg, 2 mmol) and methyl isocyanate (114 mg, 2 mmol). The solution is stirred for 5 hours at room temperature. Methylene chloride (100 mL) is added and the resulting mixture is washed with NaHCO₃ (saturated aqueous), H₂O, dried over Na₂SO₄ and the solvent removed under reduced pressure to a crude residue which is purified over silica to afford 145 mg (61% yield) of the desired product. ¹H-NMR (300 MHz, CDCl₃) δ 7.22 (d, 2H, J=8.4 Hz), 7.10 (d, 2H, J=8.1 Hz), 7.03 (d, 2H, J=8.4 Hz), 6.84 (d, 2H, J=8.8 Hz), 4.53 (s, 1H), 4.45 (m, 1H), 3.94-3.87 (m, 3H), 3.82 (s, 3H), 3.79 (m, 1H), 3.19 (m, 1H), 2.84 (d, 3H, J=4.8 Hz), 2.72 (m, 2H), 2.50 (m, 1H), 2.02 (s, 3H), 1.95 (m, 1H), 1.67 (m, 3H), 1.22 (m, 1H), 1.03 (m, 2H), 0.76 (m, 2H); ¹³C-NMR (75 MHz, CDCl₃) δ 176.0; 158.7, 158.6, 146.1, 134.4, 130.5, 130.2, 128.9, 126.1, 114.1, 79.8, 60.4, 55.7, 41.1, 40.5, 40.2, 32.9, 32.5, 30.4, 28.1, 26.3, 15.6, 10.0, 9.9; MS MH⁺=477.3; elemental analysis: theory C₂₈H₃₆N₄O₃+0.5H₂O C, 69.25, H, 7.68; N, 11.54. found C, 69.14; H, 7.56; N, 11.63.

For exemplary compounds of formula (XI) wherein both of R⁵ or R⁶ are C₁-C₄ linear, branched or cyclic alkyl or R⁵ or R⁶ are taken together to form a C₃-C₇ cyclic alkyl ring can be made by the procedure provided in Example 5. The skilled practitioner will know how to substitute the appropriate reagents, starting materials and purification methods known to those skilled in the art, in order to prepare the compounds provided herein.

### EXAMPLE 5

### Compound 9: 2-(4-Cyclopropylphenyl)-3-[2-(4-methoxyphenyl)ethyl]-1-methyl-8-(piperidine-1-carbonyl)-1,3,8-triaza-spiro[4.5]decan-4-one

Preparation of 2-(4-cyclopropylphenyl)-3-[2-(4-methoxyphenyl)ethyl]-1-methyl-8-(piperidine-1-carbonyl)-1,3,8-triaza-spiro[4.5]decan-4-one: To a solution of 2-(4-cyclopropylphenyl)-3-[2-(4-methoxyphenyl)ethyl]-1-methyl-1,3,8-triazaspiro[4.5]decan-4-one trifluoroacetate, 5, (0.13 g, 0.25 mmol) in CH₂Cl₂ (5.0 mL) is added triethylamine (0.09 mL, 0.65 mmol) and 1-piperidinecarbonyl chloride (0.04 mL, 0.32 mmol). The reaction mixture is stirred at room temperature for 20 hours. The crude material is purified over silica to afford 0.1 g of the desired product. ¹H NMR (CDCl₃) δ 7.20 (d, 2H, J=8.0 Hz), 7.07 (d, 2H, J=8.1 Hz), 7.03 (d, 2H, J=8.6 Hz), 6.81 (d, 2H, J=8.5 Hz), 4.51 (s, 1H), 3.90-3.83 (m, 2H), 3.78 (s, 3H), 3.62-3.57 (m, 2H), 3.21-3.16 (m, 5H), 2.74-2.65 (m, 2H), 2.56-2.42 (m, 1H), 2.02 (s, 3H), 1.73-1.57 (m, 9H), 1.14-1.10 (m, 1H), 1.02-0.97 (m, 2H), 0.75-0.69 (m, 2H); ESI-MS (m/z): (M+H⁺) 531.

The following are further non-limiting examples of compounds of formula (XI).

### Compound 10: 2-[4-(Diethylamino)phenyl]-3-[2-(4-methoxyphenyl)ethyl]-1-methyl-4-oxo-1,3,8-triaza-spiro[4.5]decane-8-carboxylic acid amide

¹H-NMR (300 MHz, CDCl₃) δ 7.16 (d, 2H, J=8.4 Hz), 7.08 (d, 2H, J=8.5 Hz), 6.83 (d, 2H, J=8.4 Hz), 6.68 (d, 2H, J=8.6 Hz), 4.58 (s, 2H), 4.49 (s, 1H), 4.01 (m, 1H), 3.90-3.62 (m, 3H), 3.80 (s, 3H), 3.40 (q, 4H, J=6.9 Hz, J=13.9 Hz), 3.35 (m, 1H), 2.77 (m, 2H), 2.50 (m, 1H), 2.05 (s, 3H), 1.88-1.65 (m, 3H), 1.20 (t, 6H, J=7.0 Hz); ¹³C-NMR (75 MHz, CDCl₃) δ 175.0; 158.6, 158.4, 149.1, 130.8, 130.2, 130.0, 123.0, 114.1, 111.6, 79.9, 77.6, 60.2, 55.7, 44.7, 41.6, 40.5, 33.0, 32.6, 30.4, 26.1, 12.9; MS MH⁺=494.3; elemental analysis: theory C₂₈H₃₉N₅O₃+1.0H₂O C, 65.73; H, 8.08; N, 13.69. found C, 65.50; H, 7.82; N, 13.67.

### Compound 11: 2-(4-Trifluoromethylphenyl)-3-[2-(4-methoxyphenyl)ethyl]-1-methyl-4-oxo-1,3,8-triaza-spiro[4.5]decane-8-carboxylic acid amide

¹H-NMR (300 MHz, CDCl₃) δ 7.69 (d, 2H, J=7.8 Hz), 7.47 (d, 2H, J=7.9 Hz), 7.07 (d, 2H, J=8.5 Hz), 6.86 (d, 2H, J=8.4 Hz), 4.61 (s, 1H), 4.52 (s, 2H), 3.99-3.87 (m, 3H), 3.81 (s, 3H), 3.73 (m, 1H), 3.28 (m, 1H), 2.78 (m, 1H), 2.65-2.52 (m, 2H), 2.04 (s, 3H), 1.80-1.65 (m, 3H), 1.22 (m, 1H); ¹³C-NMR (75 MHz, CDCl₃) δ 175.0; 158.8, 158.3, 141.8, 130.2, 129.4, 126.0, 114.2, 79.5, 77.6, 60.3, 55.7, 41.5, 40.6, 40.3, 33.0, 32.6, 30.4, 26.5; MS MH⁺=491.1; elemental analysis: theory C₂₅H₂₉F₃N₄O₃ C, 61.21; H, 5.96; N, 11.42. found C, 61.42; H, 6.09; N, 11.48.

### Compound 12: 2-(4-tert-Butylphenyl)-3-[2-(4-methoxyphenyl)ethyl]-1-methyl-4-oxo-1,3,8-triaza-spiro[4.5]decane-8-carboxylic acid amide

¹H-NMR (300 MHz, CDCl₃) δ 7.44 (d, 2H, J=8.3 Hz), 7.28 (d, 2H, J=8.3 Hz), 7.06 (d, 2H, J=8.4 Hz), 6.84 (d, 2H, J=8.4 Hz), 4.57 (s, 1H), 4.45 (s, 2H), 4.07 (m, 1H), 3.87 (m, 3H), 3.80 (s, 3H), 3.24 (m, 1H), 2.76 (m, 2H), 2.53 (m, 1H), 2.06 (s, 3H), 1.80 (m, 2H), 1.65 (m, 1H), 1.36 (s, 9H), 1.23 (m, 1H); ¹³C-NMR (75 MHz, CDCl₃) δ 175.0; 158.7, 158.3, 153.1, 134.3, 130.5, 130.2, 128.6, 125.9, 114.1, 79.8, 60.3, 55.7, 41.6, 40.5, 40.4, 35.1, 32.9, 32.6, 31.7, 30.4, 26.3; MS MH⁺=479.1; elemental analysis: theory C₂₈H₃₈N₄O₃+0.7H₂O C, 68.46; H, 8.08; N, 11.41. found C, 68.22; H, 7.79; N, 11.30.

### Compound 13: 2-(4-Difluoromethoxyphenyl)-3-[2-(4-methoxyphenyl)ethyl]-1-methyl-4-oxo-1,3,8-triaza-spiro[4.5]decane-8-carboxylic acid amide

¹H-NMR (300 MHz, CDCl₃) δ 7.39 (d, 2H, J=8.4 Hz), 7.18 (d, 2H, J=8.6 Hz), 7.06 (d, 2H, J=8.6 Hz), 6.85 (d, 2H, J=8.4 Hz), 6.57, 6.33 (s, s, 1H), 4.56 (m, 3H), 3.99 (m, 1H), 3.91 (m, 2H), 3.80 (s, 3H), 3.24 (m, 3H), 2.81 (m, 1H), 2.70 (m, 1H), 2.53 (m, 1H), 2.04 (s, 3H), 1.91 (m, 4H), 1.23 (m, 1H); ¹³C-NMR (75 MHz, CDCl₃) δ 175.0; 158.8, 158.3, 152.4, 134.7, 130.5, 130.3, 130.2, 120.0, 119.5, 116.0, 114.2, 112.5, 79.4, 60.3, 55.7, 41.5, 40.6, 33.0, 32.6, 30.4, 26.4; MS MH⁺=489.0; elemental analysis: theory C₂₅H₃₀F₂N₄O₄+0.5H₂O C, 60.35, H, 6.28; N, 11.26. found C, 60.63; H, 6.08; N, 11.20.

### Compound 14: 2-(4-Cyclopropylphenyl)-3-[2-(4-trifluoromethoxyphenyl)ethyl]-1-methyl-4-oxo-1,3,8-triaza-spiro[4.5]decane-8-carboxylic acid amide

¹H-NMR (300 MHz, CDCl₃) δ 7.20 (d, 2H, J=7.3 Hz), 7.10 (m, 6H), 4.79 (br, 2H), 4.56 (s, 1H), 3.88 (m, 4H), 3.22 (m, 1H), 2.78 (m, 2H), 2.56 (m, 1H), 2.04 (s, 3H), 1.93 (m, 1H), 1.73 (m, 3H), 1.19 (m, 1H), 1.01 (m, 2H), 0.74 (m, 2H); MH⁺=517.2; elemental analysis: theory C₂₇H₃₁F₃N₄O₃+0.72 mol H₂O C, 61.24; H, 6.17; N, 10.58. found C, 61.25; H, 5.88; N, 10.32.

### Compound 15: 2-(4-tert-Butylphenyl)-3-[2-(4-methoxyphenyl)ethyl]-1-methyl-4-oxo-1,3,8-triaza-spiro[4.5]decane-8-carboxylic acid ethyl amide

¹H-NMR (300 MHz, CDCl₃) δ 7.45 (d, 2H, J=8.3 Hz), 7.29 (d, 2H, J=8.3 Hz), 7.06 (d, 2H, J=8.4 Hz), 6.85 (d, 2H, J=8.7 Hz), 4.62 (s, 1H), 3.97-3.84 (m, 3H), 3.81 (s, 3H), 3.72 (m, 1H), 3.32 (q, 2H, J=7.4 Hz, J=14.5 Hz), 3.24 (m, 1H), 2.78 (m, 2H), 2.54 (m, 1H), 2.08 (s, 3H), 1.73 (m, 3H), 1.36 (s, 9H), 1.25 (m, 1H), 1.18 (t, 3H, J=7.2 Hz); ¹³C-NMR (75 MHz, CDCl₃) δ 175.0; 158.7, 158.2, 153.4, 133.4, 130.4, 130.2, 128.7, 126.0, 114.2, 79.8, 60.7, 55.7, 41.1, 40.7, 40.2, 36.3, 35.1, 32.9, 32.2, 31.7, 30.5, 26.3, 15.8; MS MH⁺=507.2; HRMS: theory C₃₀H₄₂N₄O₃ 507.3335; found 507.3319.

### Compound 16: 2-(4-Cyclopropylphenyl)-3-[2-(4-methoxyphenyl)ethyl]-1-methyl-4-oxo-1,3,8-triaza-spiro[4.5]decane-8-carboxylic acid isopropylamide

¹H-NMR (300 MHz, CDCl₃) δ 7.28 (d, 2H, J=8.9 Hz), 7.11 (d, 2H, J=8.2 Hz), 7.06 (d, 2H, J=8.2 Hz), 6.82 (d, 2H, J=8.6 Hz), 4.56 (s, 1H), 4.02 (m, 1H), 3.98 (m, 4H), 3.81 (s, 3H), 3.69 (m, 1H), 3.51 (m, 1H), 3.15 (m, 1H), 2.74 (m, 2H), 2.49 (m, 1H), 2.05 (s, 3H), 1.95 (m, 1H), 1.71 (m, 2H), 1.24 (m, 1H), 1.95 (s, s, 6H), 1.04 (m, 2H), 0.73 (m, 2H); ¹³C-NMR (75 MHz, CDCl₃) δ 176.0; 158.7, 157.5, 146.3, 133.9, 130.5, 130.2, 128.9, 126.2, 114.2, 79.8, 60.5, 55.7, 43.1, 41.1, 40.6, 40.2, 32.9, 30.4, 26.2, 23.8, 15.6, 10.1, 10.0; MS MH⁺=505.3; elemental analysis: theory C₃₀H₄₀N₄O₃+0.4 CF₃COOH C, 67.23; H, 7.40; N, 10.18. found C, 67.57; H, 7.42; N, 10.23.

### Compound 17: 2-(4-Methoxyphenyl)-3-[2-(4-methoxyphenyl)ethyl]-1-methyl-4-oxo-1,3,8-triaza-spiro[4.5]decane-8-carboxylic acid isopropylamide

¹H-NMR (300 MHz, CDCl₃) δ 7.21 (d, 2H, J=7.0 Hz), 6.99 (d, 2H, J=7.0 Hz), 6.87 (d, 2H, 8.6 Hz), 6.77 (d, 2H, J=8.5 Hz), 4.49 (s, 1H), 4.45 (br, 1H), 3.91 (m, 4H), 3.80 (s, 3H), 3.75 (s, 3H), 3.14 (m, 1H), 2.72 (m, 2H), 2.47 (m, 1H), 1.99 (s, 3H), 1.66 (m, 3H), 1.21 (m, 1H), 1.13 (s, 3H), 1.11 (s, 3H); MH⁺=495.3; elemental analysis: theory C₂₈H₃₈N₄O₄+4.64 mol H₂O C, 58.16; H, 8.24; N, 9.67. found C, 58.16; H, 8.11; N, 9.46.

### Compound 18: 2-(4-Cyclopropylphenyl)-3-[2-(4-methoxyphenyl)ethyl]-1-methyl-4-oxo-1,3,8-triaza-spiro[4.5]decane-8-carboxylic acid dimethylamide

¹H NMR (CDCl₃) δ 7.20 (d, 2H, J=8.1 Hz), 7.07 (d, 2H, J=8.1 Hz), 7.03 (d, 2H, J=8.6 Hz), 6.81 (d, 2H, J=8.6 Hz), 4.51 (s, 1H), 4.00-3.81 (m, 2H), 3.78 (s, 3H), 3.63-3.58 (m, 2H), 3.22-3.11 (m, 1H), 2.80 (s, 6H), 2.77-2.63 (m, 2H), 2.56-2.42 (m, 1H), 2.02 (s, 3H), 1.93-1.88 (m, 1H), 1.74-1.67 (m, 3H), 1.15-1.11 (m, 1H), 1.01-0.97 (m, 2H), 0.74-0.69 (m, 2H); ESI-MS (m/z): (M+H⁺) 491.

### Compound 19: 2-(4-Cyclopropylphenyl)-3-[2-(4-methoxyphenyl)ethyl]-1-methyl-4-oxo-1,3,8-triaza-spiro[4.5]decane-8-carboxylic acid diethylamide

¹H NMR (CDCl₃) δ7.20 (d, 2H, J=8.0 Hz), 7.07 (d, 2H, J=8.2 Hz), 7.04 (d, 2H, J=8.6 Hz), 6.81 (d, 2H, J=8.5 Hz), 4.52 (s, 1H), 3.90-3.83 (m, 2H), 3.78 (s, 3H), 3.60-3.55 (m, 2H), 3.22-3.13 (m, 5H), 2.74-2.63 (m, 2H), 2.51-2.47 (m, 1H), 2.03 (s, 3H), 1.93-1.89 (m, 1H), 1.73-1.68 (m, 3H), 1.14-1.09 (m, 7H), 1.03-0.97 (m, 2H), 0.74-0.69 (m, 2H); ESI-MS (m/z): (M+H⁺) 519.

### Compound 20: 2-(4-Cyclopropylphenyl)-3-[2-(4-methoxyphenyl)ethyl]-1-methyl-4-oxo-1,3,8-triaza-spiro[4.5]decane-8-carboxylic acid cyclopentylamide

¹H-NMR (300 MHz, CDCl₃) δ 7.23 (d, 2H, J=8.0 Hz), 7.11 (d, 2H, J=8.4 Hz), 7.06 (d, 2H, J=8.6 Hz), 6.82 (d, 2H, J=8.6 Hz), 4.53 (s, 1H), 4.39 (m, 1H), 4.13 (m, 1H), 3.96 (m, 1H), 3.91 (m, 2H), 3.81 (s, 3H), 3.69 (m, 1H), 3.18 (m, 1H), 3.17 (m, 2H), 2.74 (m, 1H), 2.66 (m, 3H), 2.65 (m, 3H), 1.76-1.42 (m, 6H), 1.40 (m, 3H), 1.22 (m, 1H), 1.01 (m, 2H), 0.73 (m, 2H); ¹³C-NMR (75 MHz, CDCl₃) δ 176.0; 158.7, 157.8, 146.3, 134.4, 130.5, 130.2, 128.9, 126.1, 114.1, 79.9, 60.4, 55.7, 52.9, 52.5, 41.1, 40.5, 40.1, 34.0, 32.9, 32.4, 30.4, 26.2, 24.0, 23.9, 15.6, 10.0, 9.9; MS MH⁺=531.3; elemental analysis: theory C₃₂H₄₂N₄O₃+0.5H₂O C. 71.21; H, 8.03; N, 10.38. found C, 71.13; H, 8.21; N, 10.68.

For exemplary compounds of formula (XI) wherein R⁵ and R⁶ are taken together to form a ring having 4 atoms the following procedure can be used. The skilled practitioner will know how to substitute the appropriate reagents, starting materials and purification methods known to those skilled in the art, in order to prepare the compounds provided herein.

### Compound 21: 2-(4-cyclopropylphenyl)-3-[2-(4-methoxyphenyl)ethyl]-1-methyl-8-(azetidin-1-ylcarbonyl)-1,3,8-triaza-spiro[4.5]decan-4-one

To a solution of 2-(4-cyclopropylphenyl)-3-[2-(4-methoxyphenyl)ethyl]-1-methyl-1,3,8-triazaspiro[4.5]decan-4-one trifluoroacetate, 5, (0.12 g, 0.22 mmol) in CH₂Cl₂ (5.0 mL) at 0° C. is added diisopropylethyl amine (0.10 mL, 0.57 mmol) and trichloromethyl chloroformate (25 μL, 0.21 mmol). The reaction mixture is stirred at 0° C. for 45 minutes then at room temperature for 45 minutes followed by re-cooling the reaction to 0° C. after which azetidine (0.25 g, 4.38 mmol) is added. The reaction mixture stirred with warming to room temperature for 68 hours. The crude material is purified over silica to afford 0.08 g of the desired product. ¹H NMR (CDCl₃) δ 7.20 (d, 2H, J=8.1 Hz), 7.07 (d, 2H, J=8.2 Hz), 7.03 (d, 2H, J=8.6 Hz), 6.81 (d, 2H, J=8.6 Hz), 4.51 (s, 1H), 4.03-3.93 (m, 4H), 3.86-3.82 (m, 2H), 3.78 (s, 3H), 3.75-3.63 (m, 2H), 3.20-3.06 (m, 1H), 2.80-2.59 (m, 2H), 2.55-2.42 (m, 1H), 2.28-2.14 (m, 2H), 2.00 (s, 3H), 1.96-1.85 (m, 1H), 1.79-1.51 (m, 3H), 1.18-1.07 (m, 1H), 1.02-0.98 (m, 2H), 0.75-0.71 (m, 2H); ESI-MS (m/z): (M+H⁺) 503.

### Compound 22: 2-(4-Cyclopropylphenyl)-3-[2-(4-methoxyphenyl)ethyl]-1-methyl-8-[(4-methylpiperazin-1-yl)carbonyl]-1,3,8-triaza-spiro[4.5]decan-4-one

¹H NMR (CDCl₃) δ 7.21 (d, 2H, J=8.1 Hz), 7.09 (d, 2H, J=8.1 Hz), 7.05 (d, 2H, J=8.7 Hz), 6.82 (d, 2H, J=8.7 Hz), 4.53 (s, 1H), 4.08-3.82 (m, 2H), 3.80 (s, 3H), 3.65-3.60 (m, 2H), 3.30-3.16 (m, 6H), 2.78-2.62 (m, 2H), 2.56-2.43 (m, 1H), 2.34 (s, 3H), 2.03 (s, 3H), 1.96-1.90 (m, 1H), 1.75-1.62 (m, 3H), 1.23-1.21 (m, 2H), 1.16-1.12 (m, 2H), 1.05-0.98 (m, 2H), 0.76-0.71 (m, 2H); ESI-MS (m/z): (M+H⁺) 546.

### Compound 23: 2-(4-Cyclopropylphenyl)-3-[2-(4-methoxyphenyl)ethyl]-1-methyl-8-(pyrrolidin-1-ylcarbonyl)-1,3,8-triaza-spiro[4.5]decan-4-one

¹H NMR (CDCl₃) δ7.20 (d, 2H, J=8.1 Hz), 7.08 (d, 2H, J=8.1 Hz), 7.04 (d, 2H, J=8.7 Hz), 6.81 (d, 2H, J=8.7 Hz), 4.52 (s, 1H), 4.12-3.82 (m, 2H), 3.78 (s, 3H), 3.71-3.67 (m, 2H), 3.41-3.32 (m, 5H), 3.22-3.14 (m, 1H), 2.78-2.62 (m, 2H), 2.55-2.48 (m, 1H), 2.03 (s, 3H), 1.94-1.67 (m, 7H), 1.17-1.12 (m, 1H), 1.03-0.97 (m, 2H), 0.75-0.70 (m, 2H); ESI-MS (m/z): (M+H⁺) 517.

### Compound 24: 2-(4-Cyclopropylphenyl)-3-[2-(4-methoxyphenyl)ethyl]-1-methyl-8-(morpholin-4-ylcarbonyl)-1,3,8-triaza-spiro[4.5]decan-4-one

¹H NMR (CDCl₃) δ 7.19 (d, 2H, J=8.0 Hz), 7.07 (d, 2H, J=8.1 Hz), 7.03 (d, 2H, J=8.6 Hz), 6.80 (d, 2H, J=8.5 Hz), 4.51 (s, 1H), 4.00-3.80 (m, 2H), 3.77 (s, 3H), 3.72-3.55 (m, 5H), 3.23-3.09 (m, 4H), 2.73-2.65 (m, 2H), 2.55-2.42 (m, 1H), 2.01 (s, 3H), 1.92-1.89 (m, 1H), 1.72-1.64 (m, 3H), 1.20-1.09 (m, 3H), 1.02-0.96 (m, 2H), 0.74-0.70 (m, 2H); ESI-MS (m/z): (M+H⁺) 533.

### EXAMPLE 6

Example 6 outlines the preparation of exemplary compounds according to the present invention wherein R³ is —C(O)NR⁵(OR⁷). The skilled practitioner will know how to substitute the appropriate reagents, starting materials and purification methods known to those skilled in the art, in order to prepare the compounds provided herein

### Compound 25: 2-(4-cyclopropylphenyl)-N-methoxy-3-[2-(4-methoxyphenyl)ethyl]-1-methyl-4-oxo-1,3,8-triazaspiro[4.5]decane-8-carboxamide

Preparation of 2-(4-cyclopropylphenyl)-N-methoxy-3-[2-(4-methoxyphenyl)ethyl]-1-methyl-4-oxo-1,3,8-triazaspiro[4.5]decane-8-carboxamide: To the solution of 2-(4-cyclopropylphenyl)-3-[2-(4-methoxyphenyl)ethyl]-1-methyl-1,3,8-triazaspiro[4.5]-decan-4-one, 5, (100 mg, 0.19 mmol) and Et₃N (66 μL, 0.47 mmol) in CH₂Cl₂ (2 mL) at 0° C. is added triphosgene (31 mg, 0.1 mmol). The resulting solution is stirred at 0° C. for 15 minutes then at room temperature for 1 hour. The mixture is re-cooled to 0° C. and added dropwise to a cold mixture of O-methyl hydroxylamine hydrochloride (207 mg, 2.5 mmol) and Et₃N (500 μL, 3.6 mmol) in CH₂Cl₂ (2 mL). The resulting mixture is stirred at room temperature for 3.5 days followed by stirring at 40° C. overnight. The mixture is diluted with ethyl acetate and washed with water, saturated NH₄Cl, and brine. The organic layer is dried over Na₂SO₄ and the solvent is removed under reduced pressure. The crude material is purified over silica (gradient hexanes/2-propanol 100:0 to 80:20) to afford 41 mg of the desired product as a white amorphous powder. ¹H NMR (300 MHz, CDCl₃) δ 7.51 (s, 1H), 7.18 (d, 2H, J=8.1 Hz), 7.06 (d, 2H, J=8.1 Hz), 7.02 (d, 2H, J=8.7 Hz), 6.80 (d, 2H, J=8.4 Hz), 4.50 (s, 1H), 3.84 (m, 4H), 3.77 (s, 3H), 3.70 (s, 3H), 3.17 (m, 1H), 2.69 (m, 2H), 2.49 (m, 1H), 2.00 (s, 3H), 1.90 (m, 1H), 1.69 (m, 3H), 1.17 (m, 1H), 0.99 (m, 2H), 0.71 (m, 2H); ¹³C NMR (75 MHz, CDCl₃) δ 174.9, 159.1, 158.5, 146.0, 134.0, 130.3, 130.0, 128.6, 125.9, 113.9, 79.6, 64.2, 60.1, 55.4, 40.6, 40.3, 39.9, 32.7, 32.3, 30.1, 26.1, 15.4, 9.8, 9.7; (MH⁺) 493.

### Compound 26: 2-(4-Cyclopropylphenyl)-N-hydroxy-3-[2-(4-methoxyphenyl)ethyl]-1-methyl-4-oxo-1,3,8-triazaspiro[4.5]decane-8-carboxamide

¹H NMR (300 MHz, CDCl₃) δ 7.34 (bs, 1H), 7.19 (d, 2H, J=8.1 Hz), 7.07 (d, 2H, J=8.1 Hz), 7.03 (d, 2H, J=8.4 Hz), 6.81 (d, 2H, J=8.7 Hz), 4.52 (s, 1H), 3.83 (overlapping m and s, 8H), 3.21 (m, 1H), 2.70 (m, 2H), 2.50 (m, 1H), 2.00 (s, 3H), 1.91 (m, 1H), 1.71 (m, 3H), 1.18 (m, 1H), 1.00 (m, 2H), 0.72 (m, 2H); ¹³C NMR (75 MHz, CDCl₃) δ 174.8, 161.2, 158.5, 146.0, 133.9, 130.3, 130.0, 128.7, 126.0, 113.9, 79.6, 60.0, 55.5, 40.3, 39.8, 32.7, 32.1, 30.1, 26.0, 15.4, 9.8, 9.7; (MH⁺) 478.

### Compound 27: 2-(4-cyclopropylphenyl)-N-ethoxy-3-(4-methoxyphenethyl)-1-methyl-4-oxo-1,3,8-triazaspiro[4.5]decane-8-carboxamide

¹H NMR (300 MHz, CDCl₃) δ 7.37 (bs, 1H), 7.19 (d, 2H, J=7.8 Hz), 7.07 (d, 2H, J=8.4 Hz), 7.02 (d, 2H, J=8.7 Hz), 6.80 (d, 2H, J=8.7 Hz), 4.51 (s, 1H), 3.87 (overlapping m and s, 9H), 3.18 (m, 1H), 2.69 (m, 2H), 2.49 (m, 1H), 2.00 (s, 3H), 1.91 (m, 1H), 1.68 (m, 3H), 1.23 (m, 4H), 1.00 (m, 2H), 0.71 (m, 2H); ¹³C NMR (75 MHz, CDCl₃) δ 174.9, 159.4, 158.5, 146.0, 134.0, 130.3, 130.0, 128.7, 125.9, 113.9, 79.6, 71.8, 60.1, 55.4, 40.7, 40.3, 40.0, 32.7, 32.3, 30.1, 26.1, 15.4, 13.7, 9.8, 9.7; (MH⁺) 507.

### Compound 28: 2-(4-Cyclopropylphenyl)-N-methoxy-3-[2-(4-methoxyphenyl)ethyl]-N,1-dimethyl-4-oxo-1,3,8-triazaspiro[4.5]decane-8-carboxamide

¹H NMR (300 MHz, CDCl₃) δ 7.20 (d, 2H, J=8.4 Hz), 7.07 (d, 2H, J=8.1 Hz), 7.03 (d, 2H, J=8.4 Hz), 6.81 (d, 2H, J=8.7 Hz), 4.52 (s, 1H), 3.96-3.79 (overlapping m, 4H), 3.78 (s, 3H), 3.58 (s, 3H), 3.23 (m, 1H), 2.95 (s, 3H), 2.70 (m, 2H), 2.49 (m, 1H), 2.02 (s, 3H), 1.96-1.59 (overlapping m, 4H), 1.16 (m, 1H), 1.00 (m, 2H), 0.72 (m, 2H); ¹³C NMR (75 MHz, CDCl₃) δ 175.1, 162.3, 158.5, 145.9, 134.2, 130.3, 130.0, 128.7, 126.0, 113.9, 79.6, 60.3, 58.9, 55.4, 42.3, 41.5, 40.3, 37.0, 32.7, 30.2, 26.3, 15.4, 9.9, 9.8; (MH⁺) 507.

### EXAMPLE 7

Example 7 herein below outline the preparation of exemplary compounds according to the present invention wherein R³ is —C(O)NR⁵NR⁸R⁹. The skilled practitioner will know how to substitute the appropriate reagents, starting materials and purification methods known to those skilled in the art, in order to prepare the compounds provided herein

### Compound 29: tert-Butyl 2-({2-(4-cyclopropylphenyl)-3-[2-(4-methoxyphenyl)ethyl]-1-methyl-4-oxo-1,3,8-triazaspiro[4.5]dec-8-yl}carbonyl)hydrazine carboxylate

Preparation of tert-butyl 2-({2-(4-cyclopropylphenyl)-3-[2-(4-methoxyphenyl)-ethyl]-1-methyl-4-oxo-1,3,8-triazaspiro[4.5]dec-8-yl}carbonyl)hydrazinecarboxylate: To a solution of 2-(4-cyclopropylphenyl)-3-[2-(4-methoxyphenyl)ethyl]-1-methyl-1,3,8-triazaspiro[4.5]decan-4-one, 5, (0.12 g, 0.23 mmol) in CH₂Cl₂ (5.0 mL) at 0° C. is added diisopropylethyl amine (0.10 mL, 0.57 mmol) and trichloromethyl chloroformate (25 μL, 0.21 mmol). The reaction mixture is stirred at 0° C. for 45 minutes and at room temperature for 2 hours followed by re-cooling to 0° C. and addition of tert-butylcarbazate (0.05 g, 0.35 mmol). The cooling bath is removed and the reaction is stirred for 19 hours after which time the reaction mixture is adsorbed onto silica and washed with solvent to afford 0.09 g of the desired product. ¹H NMR (CDCl₃) δ7.01 (d, 2H, J=7.9 Hz), 6.89 (d, 2H, J=7.9 Hz), 6.84 (d, 2H, J=8.3 Hz), 6.62 (d, 2H, J=8.3 Hz), 6.21 (bs, 2H), 4.34 (s, 1H), 3.78-3.59 (m, 4H), 3.09-2.93 (m, 3H), 2.58-2.45 (m, 2H), 2.38-2.24 (m, 1H), 1.84 (s, 3H), 1.75-1.71 (m, 1H), 1.63-1.49 (m, 3H), 1.28 (s, 9H), 1.09-0.97 (m, 2H), 0.85 (m, 2H), 0.56-0.51 (m, 2H); ESI-MS (m/z): (M+H⁺) 578.

### EXAMPLE 8

Exemplary Compounds of formula (XIV) can be prepared by the procedures and examples outlined in example 8. The skilled practitioner will know how to substitute the appropriate reagents, starting materials and purification methods known to those skilled in the art, in order to prepare the compounds provided herein.

### Compound 30: N′-cyano-2-(4-methoxyphenyl)-3-[2-(4-methoxyphenyl)ethyl]-1-methyl-4-oxo-1,3,8-triazaspiro[4.5]decane-8-carboximidamide

Preparation of 8-(phenyl-N-cyano-1-carbimidate)-2-(4-methoxyphenyl) —3-[2-(4-methoxyphenyl)ethyl]-1-methyl-1,3,8-triaza-spiro[4.5]decan-4-one: To the solution of 2-(4-methoxyphenyl)-3-[2-(4-methoxyphenyl)ethyl]-1-methyl-1,3,8-triaza-spiro[4.5]decan-4-one (202 mg, 0.49 mmol) in 10 mL of iso-propanol is added diphenyl cyanocarbodiimide (235 mg, 0.99 mmol), and triethylamine (0.15 mL) via syringe. The reaction is then stirred at 80° C. for 40 hours. The solvent is removed in vacuo and the resulting residue purified over silica to afford 203 mg (74% yield) of the desired product. ¹H-NMR (300 MHz, CDCl₃) δ 7.45 (m, 2H), 7.29 (m, 3H), 7.11 (m, 4H), 6.95 (d, 2H, J=8.3 Hz), 6.83 (d, 2H, J=4.79 Hz), 4.56 (s, 1H), 4.16 (m, 2H), 3.86 (s, 3H), 3.79 (m, 1H), 3.64 (s, 3H), 3.46 (m, 1H), 2.75 (m, 2H), 2.55 (m, 1H), 2.08 (s, 3H), 1.85 (m, 3H), 1.29 (m, 2H); MH⁺=554.3; elemental analysis: theory C₃₂H₃₅N₅O₄+4.55 mol H₂O C, 60.46; H, 6.99; N, 11.01. found C, 60.46; H, 6.68; N, 10.89.

Preparation of N′-cyano-2-(4-methoxyphenyl)-3-[2-(4-methoxyphenyl)ethyl]-1-methyl-4-oxo-1,3,8-triazaspiro[4.5]decane-8-carboximidamide: To a solution of the ammonia (7M in MeOH, 2.5 mL, 17.5 mmol) in a 2.0-5.0 mL Emry's process vial equipped with a stir bar is added 8-(phenyl-N-cyano-1-carbimidate) —2-(4-methoxy-phenyl)-3-[2-(4-methoxy-phenyl)ethyl]-1-methyl-1,3,8-triaza-spiro[4.5]decan-4-one (147 mg, 0.27 mmol). The reaction mixture is then capped, stirred 30 seconds, and heated in a Biotage Initiator 60 microwave for 30 minutes at 180° C. The crude residue is then purified over silica to afford 86 mg (68% yield) of the desired product. ¹H-NMR (300 MHz, CDCl₃) δ 7.28 (d, 2H, J=8.4 Hz), 7.05 (d, 2H, J=8.2 Hz), 6.94 (d, 2H, J=8.2 Hz), 6.83 (d, 2H, J=8.2 Hz), 5.71 (s, 1H), 4.55 (br, 1H), 4.13 (m, 4H), 3.86 (s, 3H), 3.81 (s, 3H), 3.33 (m, 1H), 2.77 (m, 2H), 2.54 (m, 1H), 2.05 (s, 3H), 1.76 (m, 3H), 1.28 (m, 1H); MH⁺=477.2; elemental analysis: theory C₂₆H₃₂N₆O₃+0.43 mol H₂O C, 64.48; H, 6.84; N, 17.35. found C, 64.48; H, 6.78; N, 16.98.

The following are further non-limiting examples of compounds of formula XIV of the present invention.

### Compound 31: 8-(Cyano-1-carboxamidine)-2-(4-cyclopropylphenyl)-3-[2-(4-methoxyphenyl)-ethyl]-1-methyl-1,3,8-triaza-spiro[4.5]decan-4-one

An alternative name for this compound is (E)-N′-cyano-2-(4-cyclopropylphenyl)-3-(4-methoxyphenethyl)-1-methyl-4-oxo-1,3,8-triazaspiro[4.5]decane-8-carboximidamide

A solution of phenyl N-cyano-2-(4-cyclopropylphenyl)-3-[2-(4-methoxyphenyl)ethyl]-1-methyl-4-oxo-1,3,8-triazaspiro[4.5]-decane-8-carboximidoate (0.14 g, 0.28 mmol) in 7.0 N NH₃ in MeOH (3.5 mL) is irradiated in a Biotage Initiator microwave for 30 minutes at 150° C. The reaction mixture is adsorbed silica gel and purified by normal phase chromatography to yield 0.04 g of the desired product. ¹H NMR (300 MHz, CDCl₃) δ 7.20 (d, 2H, J=8.0 Hz), 7.09 (d, 2H, J=8.0 Hz), 7.04 (d, 2H, J=8.4 Hz), 6.82 (d, 2H, J=8.4 Hz), 5.71 (s, 2H), 4.53 (s, 1H), 4.15-3.86 (m, 3H), 3.80 (s, 3H), 3.53-3.37 (m, 1H), 2.82-2.60 (m, 2H), 2.58-2.43 (m, 1H), 2.06 (s, 3H), 1.97-1.80 (m, 2H), 1.79-1.59 (m, 2H), 1.33-1.14 (m, 2H), 1.03-0.99 (m, 2H), 0.76-0.73 (m, 2H); ESI-MS (m/z): (M+H⁺) 487.

### EXAMPLE 9

Exemplary compounds of formula (XV) can be prepared by the procedures and examples outlined herein below in Example 9. The skilled practitioner will know how to substitute the appropriate reagents, starting materials and purification methods known to those skilled in the art, in order to prepare the compounds provided herein

### Compound 32: 2-(4-cyclopropylphenyl)-8-methanesulfonyl-3-[2-(4-methoxy-phenyl)ethyl]-1-methyl-1,3,8-triaza-spiro[4.5]decan-4-one

Preparation of 2-(4-cyclopropylphenyl)-8-methanesulfonyl-3-[2-(4-methoxy-phenyl)ethyl]-1-methyl-1,3,8-triaza-spiro[4.5]decan-4-one: To a solution of the 2-(4-cyclopropylphenyl)-3-[2-(4-methoxyphenyl)ethyl]-1-methyl-1,3,8-triaza-spiro[4.5]-decan-4-one, 5, (210 mg, 0.5 mmol) in CH₂Cl₂ (10 mL) is added methanesulfonyl chloride (114 mg, 1.0 mmol), triethylamine (TEA) (200 mg, 7.3 mmol). After stirring at room temperature for 3 hours, the CH₂Cl₂ is evaporated and the residue dissolved in EtOAc (100 mL). The EtOAc layer is washed with aqueous NaHCO₃, H₂O and dried over NaSO₄. The solvent is removed in vacuo and the resulting crude material is purified by HPLC to afford 160 mg (64% yield) of the desired product. ¹H-NMR (300 MHz, CDCl₃) δ 7.23 (d, 2H, J=7.7 Hz), 7.11 (d, 2H, J=7.7 Hz), 7.05 (d, 2H, J=8.4 Hz), 6.83 (d, 2H, J=8.0 Hz), 4.56 (s, 1H), 3.80 (s, 3H), 3.76-3.68 (m, 4H), 3.09 (m, 1H), 2.83 (s, 3H), 2.72 (m, 2H), 2.56 (m, 1H), 2.06 (s, 3H), 1.91 (m, 4H), 1.24 (m, 1H), 1.04 (m, 2H), 0.75 (m, 2H); ¹³C-NMR (75 MHz, CDCl₃) δ 175.0; 158.7, 146.3, 134.1, 130.4, 130.2, 128.8, 126.2, 114.1, 79.9, 59.5, 55.7, 43.0, 42.6, 40.6, 34.7, 32.9, 32.6, 30.3, 26.6, 15.6, 10.1, 10.0; MS MH⁺=498.0; elemental analysis: theory C₂₇H₃₅N₃O₄S C, 65.16; H, 7.09; N, 8.44. found C, 65.20; H, 6.77; N, 8.37.

The following are further non-limiting examples of compounds of formula (XV) of the present invention.

### Compound 33: 2-(4-tert-Butylphenyl)-8-methanesulfonyl-3-[2-(4-methoxyphenyl)ethyl]-1-methyl-1,3,8-triaza-spiro[4.5]decan-4-one

¹H-NMR (300 MHz, CDCl₃) δ 7.45 (d, 2H, J=8.1 Hz), 7.29 (d, 2H, J=8.3 Hz), 7.04 (d, 2H, J=8.4 Hz), 6.83 (d, 2H, J=8.5 Hz), 6.70 (bs, 1H), 4.67 (s, 1H), 4.05 (m, 1H), 3.92 (m, 1H), 3.80 (s, 3H), 3.44 (m, 1H), 3.34 (m, 2H), 2.80 (m, 2H), 2.55 (m, 1H), 2.15 (s, 3H), 2.05 (m, 3H), 1.37 (s, 9H), 1.28 (m, 1H); ¹³C-NMR (75 MHz, CDCl₃) δ 175.0; 158.8, 153.5, 133.3, 130.3, 130.2, 128.6, 126.1, 114.2, 80.0, 60.0, 55.6, 42.9, 42.5, 40.9, 35.1, 34.8, 32.9, 32.4, 31.6, 30.4, 26.8; MS MH⁺=514.1; elemental analysis: theory C₂₈H₃₉N₃O₂+0.5 CF₃COOH C, 61.03; H, 6.98; N, 7.36. found C, 61.15; H, 7.01; N, 7.36.

### Compound 34: 2-(4-Trifluoromethylphenyl)-8-methanesulfonyl-3-[2-(4-methoxyphenyl)ethyl]-1-methyl-1,3,8-triaza-spiro[4.5]decan-4-one

¹H-NMR (300 MHz, CDCl₃) δ 7.70 (d, 2H, J=8.1 Hz), 7.47 (d, 2H, J=8.1 Hz), 7.06 (d, 2H, J=8.5 Hz), 6.85 (d, 2H, J=8.8 Hz), 4.63 (s, 1H), 3.89-3.82 (m, 2H), 3.81 (s, 3H), 3.79-3.66 (m, 2H), 3.11 (m, 1H), 2.84 (s, 3H), 2.79 (m, 1H), 2.66-2.54 (m, 2H), 2.07 (s, 3H), 1.94-1.85 (m, 3H), 1.31 (m, 1H); ¹³C-NMR (75 MHz, CDCl₃) δ 175.0; 158.9, 141.6, 130.2, 129.4, 126.1, 122.3, 114.2, 79.6, 77.6, 59.6, 55.7, 42.9, 42.5, 40.7, 34.8, 33.0, 32.6, 30.3, 26.8; MS MH⁺=526.1; elemental analysis: theory C₂₅H₃₀F₃N₃O₄S+0.2H₂O C, 56.74; H, 5.79; N, 7.94. found C, 56.35; H, 5.70; N, 7.63.

### EXAMPLE 10

Exemplary compounds of formula (XVI) can be prepared by the procedures and examples outlined herein below in Example 10. The skilled practitioner will know how to substitute the appropriate reagents, starting materials and purification methods known to those skilled in the art, in order to prepare the compounds provided herein

### Compound 35: Ethyl 2-(4-cyclopropylphenyl)-3-[2-(4-methoxyphenyl)ethyl]-1-methyl-4-oxo-1,3,8-triazaspiro[4.5]decane-8-carboxylate

Preparation of ethyl 2-(4-cyclopropylphenyl)-3-[2-(4-methoxyphenyl)ethyl]-1-methyl-4-oxo-1,3,8-triazaspiro[4.5]decane-8-carboxylate: To a solution of 2-(4-cyclopropylphenyl)-3-[2-(4-methoxyphenyl)ethyl]-1-methyl-1,3,8-triazaspiro[4.5]decan-4-one, 5, (0.12 g, 0.22 mmol) in CH₂Cl₂ (5.0 mL) is added triethylamine (0.09 mL, 0.65 mmol) and ethyl chloroformate (0.03 mL, 0.31 mmol). The reaction mixture is stirred at room temperature for 20 hours. The crude product is purified over silica to afford 0.08 g of the desired product. ¹H NMR (CDCl₃) δ 7.12 (d, 2H, J=8.2 Hz), 7.00 (d, 2H, J=8.1 Hz), 6.95 (d, 2H, J=8.6 Hz), 6.73 (d, 2H, J=8.6 Hz), 4.44 (s, 1H), 4.09-3.74 (m, 6H), 3.71 (s, 3H), 3.09 (bs, 1H), 2.67-2.56 (m, 2H), 2.48-2.35 (m, 1H), 1.94 (s, 3H), 1.87-1.81 (m, 1H), 1.76-1.41 (m, 3H), 1.19 (t, 3H, J=7.1 Hz), 1.13-1.03 (m, 1H), 0.94-0.91 (m, 2H), 0.67-0.62 (m, 2H); ESI-MS (m/z): (M+H⁺) 492.

The following are further non-limiting examples of compounds of formula (XVI) of the present invention.

### Compound 36: Isopropyl 2-(4-cyclopropylphenyl)-3-[2-(4-methoxyphenyl)ethyl]-1-methyl-4-oxo-1,3,8-triazaspiro[4.5]decane-8-carboxylate

¹H NMR (CDCl₃) δ 7.20 (d, 2H, J=8.1 Hz), 7.08 (d, 2H, J=8.1 Hz), 7.03 (d, 2H, J=8.6 Hz), 6.81 (d, 2H, J=8.6 Hz), 4.96-4.88 (m, 1H), 4.51 (s, 1H), 4.13-3.82 (m, 3H), 3.78 (s, 3H), 3.15 (bs, 1H), 2.79-2.63 (m, 2H), 2.56-2.42 (m, 1H), 2.02 (s, 3H), 1.95-1.89 (m, 1H), 1.81-1.47 (m, 3H), 1.25 (d, 6H, J=6.3 Hz), 1.20-1.09 (m, 1H), 1.02-0.98 (m, 2H), 0.75-0.71 (m, 2H); ESI-MS (m/z): (M+H⁺) 506.

### Compound 37: tert-Butyl 2-(4-methoxyphenyl)-3-[2-(4-methoxyphenyl)ethyl]-1-methyl-4-oxo-1,3,8-triazaspiro[4.5]decane-8-carboxylate

¹H-NMR (300 MHz, CDCl₃) δ 7.24 (d, 2H, J=8.6 Hz), 7.06 (d, 2H, J=8.2 Hz), 6.94 (d, 2H, J=8.5 Hz), 6.81 (d, 2H, J=8.2 Hz), 4.52 (s, 1H), 3.93 (m, 4H), 3.85 (s, 3H), 3.79 (s, 3H), 3.18 (m, 1H), 2.78-2.67 (m, 2H), 2.51 (m, 1H), 2.03 (s, 3H), 1.74 (m, 3H), 1.48 (s, 9H), 1.18 (m, 1H); MS MH⁺=510.2.

### Compound 38: tert-Butyl 2-(4-tert-butylphenyl)-3-[2-(4-methoxyphenyl)ethyl]-1-methyl-4-oxo-1,3,8-triazaspiro[4.5]decane-8-carboxylate:

¹H-NMR (300 MHz, CDCl₃) δ 7.45 (d, 2H, J=8.2 Hz), 7.30 (d, 2H, J=8.2 Hz), 7.07 (d, 2H, J=8.2 Hz), 6.85 (d, 2H, J=8.2 Hz), 4.57 (s, 1H), 4.00 (m, 4H), 3.82 (s, 3H), 3.18 (m, 1H), 2.83 (m, 2H), 2.56 (m, 1H), 2.07 (s, 3H), 1.77 (m, 3H), 1.50 (s, 9H), 1.37 (s, 9H), 1.15 (m, 1H); MS MH⁺=536.0.

### Compound 39: tert-Butyl 2-(4-diethylaminophenyl)-3-[2-(4-methoxyphenyl)ethyl]-1-methyl-4-oxo-1,3,8-triazaspiro[4.5]decane-8-carboxylate

¹H-NMR (300 MHz, CDCl₃) δ 7.17 (d, 2H, J=8.7 Hz), 7.09 (d, 2H, J=8.7 Hz), 6.85 (d, 2H, J=8.7 Hz), 6.66 (d, 2H, J=8.7 Hz), 4.49 (s, 1H), 4.00 (m, 4H), 3.84 (s, 3H), 3.41 (m, 4H), 3.18 (m, 1H), 2.80 (m, 2H), 2.56 (m, 1H), 2.06 (s, 3H), 1.75 (m, 3H), 1.49 (s, 9H), 1.21 (m, 6H), 1.15 (m, 1H); MS MH⁺=5551.1.

### Compound 40: tert-Butyl 2-(4-difluoromethoxyphenyl)-3-[2-(4-methoxyphenyl)ethyl]-1-methyl-4-oxo-1,3,8-triazaspiro[4.5]decane-8-carboxylate

¹H-NMR (300 MHz, CDCl₃) δ 7.37 (d, 2H, J=8.6 Hz), 7.18 (d, 2H, J=8.4 Hz), 7.07 (d, 2H, J=8.8 Hz), 6.85 (d, 2H, J=8.2 Hz), 6.58, 6.33 (s, s, 1H), 4.57 (s, 1H), 3.94 (m, 4H), 3.85 (s, 3H), 3.23 (m, 1H), 2.79 (m, 1H), 2.69 (m, 1H), 2.55 (m, 1H), 2.05 (s, 3H), 1.78 (m, 1H), 1.50 (m, 2H), 1.42 (s, 9H), 1.18 (m, 1H); MS MH⁺=547.2.

### Compound 41: Methyl 2-(4-cyclopropylphenyl)-3-[2-(4-methoxyphenyl)ethyl]-1-methyl-4-oxo-1,3,8-triazaspiro[4.5]decane-8-carboxylate

¹H-NMR (300 MHz, CDCl₃) δ 7.25 (d, 2H, J=7.7 Hz), 7.22 (d, 2H, J=7.6 Hz), 7.07 (d, 2H, J=7.1 Hz), 6.85 (d, 2H, J=7.4 Hz), 4.58 (s, 1H), 4.06 (m, 1H0, 3.94 (m, 3H), 3.81 (s, 3H), 3.78 (s, 3H), 3.73 (m, 1H), 3.21 (m, 1H), 2.79 (m, 2H), 2.57 (m, 1H), 2.03 (s, 3H), 1.79 (m, 1H), 1.61 (m, 3H), 1.21 (m, 1H), 1.04 (m, 2H), 0.73 (m, 2H); MS MH⁺=478.2.

### EXAMPLE 11

Exemplary compounds of formula (XVII) can be prepared by the procedures and examples outlined herein below in Example 11. The skilled practitioner will know how to substitute the appropriate reagents, starting materials and purification methods known to those skilled in the art, in order to prepare the compounds provided herein

### Compound 42: 2-(4-Cyclopropylphenyl)-8-(isoxazol-5-yl-carbonyl)-3-[2-(4-methoxyphenyl)ethyl]-1-methyl-1,3,8-triazaspiro[4.5]decan-4-one

Preparation of 2-(4-cyclopropylphenyl)-8-(isoxazol-5-ylcarbonyl)-3-[2-(4-methoxyphenyl)ethyl]-1-methyl-1,3,8-triazaspiro[4.5]decan-4-one: To a solution of 2-(4-cyclopropylphenyl)-3-[2-(4-methoxyphenyl)ethyl]-1-methyl-1,3,8-triazaspiro[4,5]-decan-4-one trifluoroacetate (0.13 g, 0.25 mmol) in CH₂Cl₂ (5.0 mL) is added triethylamine (0.17 mL, 1.22 mmol) and isoxazole-5-carbonyl chloride (0.04 g, 0.29 mmol). The reaction mixture is stirred at room temperature for 20 hours. The crude product is purified over silica to afford 0.05 g of the desired product. ¹H NMR (CDCl₃) δ 8.17-8.15 (m, 1H), 7.07-7.04 (m, 2H), 6.94-6.87 (m, 4H), 6.69-6.58 (m, 3H), 4.51-4.28 (m, 2H), 4.19-3.67 (m, 3H), 3.63, 3.61 (s, rotamers, 3H), 3.49-3.35, 3.14-2.99 (m, 1H), 2.69-2.45 (m, 2H), 2.43-2.26 (m, 1H), 1.89 (s, 3H), 1.83-1.45 (m, 5H), 1.13-1.08 (m, 2H), 0.89-0.82 (m, 2H), 0.60-0.55 (m, 2H); ESI-MS (m/z): (M+H⁺) 514.

Exemplary compounds of formula XVIII of the present invention can be prepared by the same procedures as outlined herein by replacing 4-methoxyphenethyl amine with 3-phenylpropyl amine. The following are non-limiting examples of compounds according of formula XVIII of the present invention. The skilled practitioner will know how to substitute the appropriate reagents, starting materials and purification methods known to those skilled in the art, in order to prepare additional compounds of the present invention.

### Compound 43: 2-(4-Cyclopropylphenyl)-1-methyl-3-(3-phenylpropyl) —1,3,8-triazaspiro[4.5]-decan-4-one

¹H-NMR (300 MHz, CDCl₃) δ 7.25 (m, 4H), 7.23 (m, 1H), 7.09 (m, 4H), 4.77 (s, 1H), 3.75 (m, 1H), 3.50 (m, 1H), 3.34 (br, 1H), 3.23 (m, 1H), 3.06 (m, 2H), 2.59 (m, 1H), 2.48 (m, 2H), 2.17 (s, 3H), 1.92 (m, 1H), 1.84 (m, 2H), 1.64 (m, 4H), 1.00 (m, 2H), 0.73 (m, 2H); ¹³C-NMR (75 MHz, CDCl₃) δ 175.5, 145.9, 141.6, 134.7, 128.7, 128.6, 128.4, 126.1, 126.0, 79.7, 60.2, 42.7, 42.2, 39.6, 33.6, 33.3, 30.5, 29.2, 27.4, 15.5, 9.9; ESI/MS MH⁺=404.1; elemental analysis: theory C₂₆H₃₃N₃O+0.23 mol H₂O C, 76.59; H, 8.27; N, 10.30. found C, 76.59; H, 8.41; N, 10.28.

### Compound 44: 8-Acetyl-2-(4-cyclopropylphenyl)-3-(3-phenylpropyl)-1-methyl-1,3,8-triazaspiro-[4.5]decan-4-one

¹H-NMR (300 MHz, CDCl₃) δ 7.23 (m, 5H), 7.08 (m, 4H), 4.78 (s, 1H), 4.59 (m, 1H), 4.23 (m, 0.5H), 3.69 (m, 2H), 3.48 (m, 1H), 3.20 (m, 0.5H), 2.65 (m, 1H), 2.45 (m, 2H), 2.14 (s, 3H), 2.11 (s, 3H), 1.95 (m, 2H), 1.66 (m, 5H), 1.00 (m, 2H), 0.80 (m, 2H); MH⁺=446.2; elemental analysis: theory C₂₈H₃₅N₃O₂+2.03 mol H₂O C, 69.75; H, 8.17; N, 8.71. found C, 69.75; H, 7.88; N, 8.63.

### Compound 45: 8-Cyclopropanecarbonyl-2-(4-cyclopropylphenyl)-3-(3-phenylpropyl)-1-methyl-1,3,8-triazaspiro-[4.5]decan-4-one

¹H-NMR (300 MHz, CDCl₃) δ 7.25 (m, 4H), 7.19 (m, 1H), 7.08 (m, 4H), 4.79 (s, 1H), 4.58 (m, 1H), 4.30 (m, 0.5H), 4.23 (m, 1H), 3.74 (m, 1H), 3.52 (m, 1H), 3.26 (m, 0.5H), 2.65 (m, 1H), 2.52 (m, 2H), 2.13 (s, 3H), 1.95 (m 2H), 1.80 (m, 4H), 1.59 (m, 2H), 1.01 (m, 4H), 0.76 (m, 4H); MH⁺=472.3; elemental analysis: theory C₃₀H₃₇N₃O₂+0.28 mol H₂O C, 75.69; H, 7.94; N, 8.82. found C, 75.60; H, 7.67; N, 8.56.

### Compound 46: 8-Cyclopropanecarbonyl-2-(4-methoxyphenyl)-3-(3-phenylpropyl)-1-methyl-1,3,8-triazaspiro-[4.5]decan-4-one

¹H-NMR (300 MHz, CDCl₃) δ 7.21 (m, 5H), 7.07 (d, 2H, J=7.3 Hz), 6.91 (d, 2H, J=8.4 Hz), 4.78 (s, 1H), 4.55 (m, 1H), 4.16 (m 1.5H), 3.85 (s, 3H), 3.78 (m, 1H), 3.50 (m, 1H), 3.24 (m, 0.5H), 2.64 (m, 1H), 2.51 (m, 2H), 2.12 (s, 3H), 1.89 (m, 1H), 1.82 (m, 3H), 1.66 (m, 3H), 1.01 (m, 2H), 0.77 (m, 2H); MH⁺=462.2; elemental analysis: theory C₂₈H₃₅N₃O₃ C, 72.86; H, 7.64; N, 9.10. found C, 72.54; H, 7.51; N, 9.23.

### Compound 47: 2-(4-Cyclopropylphenyl)-1-methyl-4-oxo-3-(3-phenylpropyl)-1,3,8-triazaspiro-[4.5]decane-8-carboxylic acid tert-butyl ester

¹H-NMR (300 MHz, CDCl₃) δ 7.23 (m, 5H), 7.07 (m, 4H, J=7.0 Hz), 4.77 (s, 1H), 4.06 (br, 2H), 3.85 (br, 1H), 3.51 (m, 1H), 3.32 (br, 1H), 2.61 (m, 1H), 2.49 (m, 2H), 2.13 (s, 3H), 1.93 (m, 1H), 1.79 (m, 4H), 1.63 (m, 2H), 1.50 (s, 9H), 1.00 (m, 2H), 0.74 (m, 2H); ¹³C-NMR (75 MHz, CDCl₃) δ 175.2, 155.3, 146.0, 141.5, 134.5, 128.7, 128.6, 128.4, 126.2, 126.1, 79.8, 79.6, 60.3, 39.6, 33.3, 32.9, 30.4, 29.2, 28.7, 26.5, 15.5, 9.9; ESI/MS MH⁺=504.2; elemental analysis: theory C₃₁H₄₁N₃O₃+0.95 mol H₂O C, 71.49; H, 8.30; N, 8.06. found C, 71.49; H, 8.36; N, 8.24.

### Compound 48: tert-butyl 2-(4-tert-butylphenyl)-4-oxo-3-(3-phenylpropyl)-1,3,8-triazaspiro[4.5]decane-8-carboxylate

¹H-NMR (300 MHz, CDCl₃) δ 7.45 (d, 2H, J=8.2 Hz), 7.26 (m, 4H), 7.17 (m, 1H), 7.05 (d, 2H, J=7.3 Hz), 5.35 (s, 1H), 4.00 (br, 2H), 3.56 (m, 1H), 3.22 (m, 1H), 2.99 (m, 1H), 2.76 (m, 1H), 2.51 (m, 2H), 2.19 (m, 1H), 1.90 (m, 2H), 1.81 (m, 2H), 1.70 (m, 2H), 1.52 (br, 1H), 1.46 (s, 9H), 1.34 (s, 9H); ¹³C-NMR (75 MHz, CDCl₃) δ 176.9, 154.8, 153.1, 141.4, 135.6, 128.6, 128.4, 127.1, 126.4, 126.2, 79.8, 74.4, 60.5, 40.6, 35.0, 34.5, 33.2, 32.0, 31.6, 28.8, 28.7; ESI/MS MH⁺=506.5.

### Compound 49: 2-(4-Cyclopropylphenyl)-1-methyl-4-oxo-3-(3-phenylpropyl)-1,3,8-triazaspiro-[4.5]decane-8-carboxylic acid amide

¹H-NMR (300 MHz, CDCl₃) δ 7.25 (m, 4H), 7.23 (m, 1H), 7.09 (m, 4H), 4.77 (s, 1H), 3.75 (m, 1H), 3.50 (m, 1H), 3.34 (br, 1H), 3.23 (m, 1H), 3.06 (m, 2H), 2.59 (m, 1H), 2.48 (m, 2H), 2.17 (s, 3H), 1.92 (m, 1H), 1.84 (m, 2H), 1.64 (m, 4H), 1.00 (m, 2H), 0.73 (m, 2H); ¹³C-NMR (75 MHz, CDCl₃) δ 174.8, 158.2, 145.6, 141.0, 134.0, 128.3, 128.2, 128.0, 125.8, 125.7, 79.4, 59.8, 41.0, 40.0, 39.2, 32.9, 32.4, 30.0, 28.8, 26.0, 15.1, 9.5; ESI/MS MH⁺=447.1; elemental analysis: theory C₂₄H₃₄N₄O₂+0.71 mol H₂O C, 70.59; H, 7.77; N, 12.19. found C, 70.57; H, 7.68; N, 12.13.

### Compound 50: 2-(4-tert-Butylphenyl)-1-methyl-4-oxo-3-(3-phenylpropyl)-1,3,8-triazaspiro-[4.5]decane-8-carboxylic acid amide

¹H-NMR (300 MHz, CDCl₃) δ 7.38 (d, 2H, J=7.0 Hz), 7.27 (d, 2H, J=7.0 Hz), 7.21 (d, 2H, J=7.5 Hz), 7.15 (m, 1H), 7.03 (d, 2H, J=7.5 Hz), 5.05 (br, 2H), 4.79 (s, 1H), 3.99 (m, 2H), 3.84 (m, 1H), 3.43 (m, 2H), 2.67 (m, 1H), 2.47 (m, 2H), 2.13 (s, 3H), 1.82 (m, 3H), 1.61 (m, 3H), 1.34 (s, 9H); ¹³C-NMR (75 MHz, CDCl₃) δ 175.2, 158.7, 152.9, 141.4, 134.4, 128.6, 128.4, 126.2, 125.8, 79.8, 60.2, 41.4, 40.4, 39.7, 35.0, 33.3, 32.8, 31.6, 30.5, 29.2, 26.5; ESI/MS MH⁺=463.6; elemental analysis: theory C₂₈H₃₆N₄O₃+0.76 mol H₂O C, 70.60; H, 8.36; N, 11.76. found C, 70.60; H, 8.24; N, 11.75.

### Compound 51: 2-(4-Cyclopropylphenyl)-1-methyl-4-oxo-3-(3-phenylpropyl)-1,3,8-triazaspiro-[4.5]decane-8-carboxylic acid methylamide

An alternative name for this compound is 2-(4-cyclopropylphenyl)-N,1-dimethyl-4-oxo-3-(3-phenylpropyl)-1,3,8-triazaspiro[4.5]decane-8-carboxamide. ¹H-NMR (300 MHz, CDCl₃) δ 7.25 (m, 5H), 7.04 (m, 4H), 4.95 (br, 1H), 4.76 (s, 1H), 4.02 (m, 2H), 3.91 (m, 2H), 3.79 (m, 2H), 3.42 (m, 2H), 2.81 (s, 3H), 2.59 (m, 1H), 2.49 (m, 2H), 2.10 (s, 3H), 1.94 (m, 1H), 1.80 (m, 3H), 1.26 (m, 1H), 0.99 (m, 2H), 0.72 (m, 2H); MH⁺=461.3; elemental analysis: theory C₂₈H₃₆N₄O₂+1.17 mol H₂O C, 69.82; H, 8.02; N, 11.63. found C, 69.82; H, 7.69; N, 11.65.

### Compound 52: 2-(4-Cyclopropylphenyl)-1-methyl-4-oxo-3-(3-phenylpropyl)-1,3,8-triazaspiro-[4.5]decane-8-carboxylic acid isopropyl amide

¹H-NMR (300 MHz, CDCl₃) δ 7.24 (m, 4H), 7.17 (m, 1H), 7.04 (m, 4H), 4.77 (s, 1H), 4.42 (br, 1H), 3.96 (m, 3H), 3.74 (m, 1H), 3.43 (m, 2H), 2.59 (m, 1H), 2.46 (m, 2H), 2.11 (s, 3H), 1.81 (m, 4H), 1.63 (m, 3H), 1.17 (s, 3H), 1.16 (s, 3H), 0.95 (m, 2H), 0.72 (m, 2H); MH⁺=489.3; elemental analysis: theory C₃₀H₄₀N₄O₂+1.14 mol H₂O C, 70.76; H, 8.36; N, 11.00. found C, 70.75; H, 8.04; N, 11.13.

### Compound 53: 2-(4-Cyclopropylphenyl)-1-methyl-4-oxo-3-(3-phenylpropyl)-1,3,8-triazaspiro-[4.5]decane-8-carboxylic acid dimethylamide

¹H-NMR (300 MHz, CDCl₃) δ 7.23 (m, 4H), 7.14 (m, 1H), 7.08 (m, 4H), 4.78 (s, 1H), 3.97 (m, 1H), 3.72 (m, 2H), 3.44 (m, 2H), 2.84 (s, 6H), 2.61 (m, 1H), 2.43 (m, 2H), 2.14 (s, 3H), 1.91 (m, 2H), 1.81 (m, 2H), 1.63 (m, 3H), 1.03 (m, 2H), 0.76 (m, 2H); MH⁺=475.3; elemental analysis: theory C₂₉H₃₈N₄O₂+1.70 mol H₂O C, 68.93; H, 8.26; N, 11.09. found C, 68.94; H, 7.93; N, 10.84.

### Compound 54: 2-(4-Cyclopropylphenyl)-8-methanesulfonyl-1-methyl-3-(3-phenylpropyl)-1,3,8-triazaspiro-[4.5]decan-4-one

¹H-NMR (300 MHz, CDCl₃) δ 7.27 (m, 4H), 7.21 (m, 1H), 7.08 (m, 4H), 4.80 (s, 1H), 3.81 (m, 3H), 3.46 (m, 1H), 3.32 (m, 1H), 2.84 (s, 3H), 2.66 (m, 1H), 2.49 (m, 2H), 2.06 (s, 3H), 2.00 (m 4H), 1.74 (m, 1H), 1.62 (m, 2H), 1.03 (m, 2H), 0.74 (m, 2H); MH⁺=482.2; elemental analysis: theory C₂₇H₃₅N₃O₃S+1.21 mol H₂O C, 64.41; H, 7.49; N, 8.35. found C, 64.41; H, 7.32; N, 8.06.

Exemplary compounds of formula XIX (L² equal to methylene, —CH₂—) can be prepared according to the examples 12 and 13 or with modifications which are routine to the artisan.

### EXAMPLE 12

### Compound 55: 2-{2-(4-Cyclopropylphenyl)-3-[2-(4-methoxyphenyl)ethyl]-1-methyl-4-oxo-1,3,8-triaza-spiro[4.5]dec-8-yl}acetamide

Preparation of 2-{2-(4-cyclopropylphenyl)-3-[2-(4-methoxyphenyl)ethyl]-1-methyl-4-oxo-1,3,8-triaza-spiro[4.5]dec-8-yl}acetamide: To a solution of 2-(4-cyclopropylphenyl)-3-[2-(4-methoxyphenyl)ethyl]-1-methyl-1,3,8-triaza-spiro[4.5]decan-4-one, (238 mg contained 0.5 mmol TFA salt, 0.5 mmol) in acetonitrile (15 mL) is added triethylamine (100 mg, 1 mmol) and 2-bromoacetamide (137 mg, 1 mmol). The resulting mixture is stirred for 3 hours at room temperature. EtOAc (100 mL) and H₂O (50 mL) are added and the layers separated. The organic layer is washed with NaHCO₃ (saturated aqueous), H₂O, dried over Na₂SO₄ and concentrated under reduced pressure to a residue which is purified over silica to afford 154 mg (65% yield) of the desired product. ¹H-NMR (300 MHz, CDCl₃) δ 7.22 (d, 2H, J=8.1 Hz), 7.15 (d, 1H, J=4.1 Hz), 7.10 (d, 2H, J=8.0 Hz), 7.04 (d, 2H, J=8.5 Hz), 6.82 (d, 2H, J=8.5 Hz), 5.73 (d, 1H, J=4.6 Hz), 4.51 (s, 1H), 3.84 (m, 1H), 3.78 (s, 3H), 3.32 (m, 1H), 3.09 (s, 2H), 2.77 (m, 5H), 2.51 (m, 1H), 2.08 (s, 3H), 1.94 (m, 1H), 1.82 (m, 3H), 1.25 (m, 1H), 1.05 (m, 2H), 0.76 (m, 2H); ¹³C-NMR (75 MHz, CDCl₃) δ 177.0; 175.0, 158.6, 146.1, 134.6, 130.7, 130.2, 128.9, 126.1, 114.1, 79.9, 61.7, 59.6, 55.7, 50.5, 50.0, 40.6, 33.1, 32.9, 30.6, 26.6, 15.6, 10.0, 9.9; MS MH⁺=477.1; elemental analysis: theory C₂₈H₃₈N₄O₃+0.2H₂O C, 70.03; H, 7.64; N, 11.67. found C, 69.84; H, 7.60; N, 11.60.

### EXAMPLE 13

### Compound 56: 8-Cyclopropylmethyl-2-(4-cyclopropylphenyl)-3-[2-(4-methoxyphenyl)ethyl]-1-methyl-1,3,8-triaza-spiro[4.5]decan-4-one

Preparation of 8-cyclopropylmethyl-2-(4-cyclopropylphenyl)-3-[2-(4-methoxy-phenyl)ethyl]-1-methyl-1,3,8-triaza-spiro[4.5]decan-4-one: To a solution of 2-(4-cyclopropylphenyl)-3-[2-(4-methoxyphenyl)ethyl]-1-methyl-1,3,8-triaza-spiro[4.5]decan-4-one, 5, (476 mg contained 0.5 mmol TFA salt, 1.0 mmol) in ClCH₂CH₂Cl (10 mL) is added cyclopropancarbaldehyde (84 mg, 1.2 mmol), glacial acetic acid (0.1 mL) and sodium triacetoxyborohydride (233 mg, 1.1 mmol). The resulting mixture is stirred for 24 hours at room temperature. The reaction mixture is diluted with CH₂Cl₂ and washed with NaHCO₃ (50 mL, saturated aqueous). The organic layer is removed and the aqueous layer extracted by CH₂Cl₂ (50 mL). The combined organic layers are washed with NaHCO₃, H₂O, dried over Na₂SO₄ and purified via HPLC to afford 293 mg (62% yield) of the desired product. ¹H-NMR (300 MHz, CDCl₃) δ 7.23 (d, 2H, J=8.2 Hz), 7.12 (d, 2H, J=8.2 Hz), 7.08 (d, 2H, J=8.6 Hz), 6.84 (d, 2H, J=8.6 Hz), 4.56 (m, 1H), 3.92 (m, 2H), 3.80 (s, 3H), 3.62 (m, 1H), 3.50 (m, 1H), 3.16 (m, 1H), 2.94 (m, 2H), 2.73 (m, 2H), 2.54 (m, 1H), 2.32 m, 2H), 2.07 (s, 3H), 1.97 (m, 2H), 1.22 (m, 2H), 1.07 (m, 2H), 0.82 (m, 4H), 0.42 (m, 2H); ¹³C-NMR (75 MHz, CDCl₃) δ 175.0; 162.5, 158.8, 146.5, 133.3, 130.3, 130.1, 128.8, 126.3, 114.7, 114.1, 79.9, 62.0, 58.4, 55.6, 49.0, 48.7, 40.3, 32.9, 30.1, 24.1, 15.6, 10.1, 10.0, 5.9, 5.0; MS MH⁺=488.3; elemental analysis: theory C₃₆H₃₉N₃O₂+1.2 CF₃COOH C, 63.75; H, 6.64; N, 6.88. found C, 63.87; H, 6.75; N, 6.76.

The following is a non-limiting example of a compound of formula XIX of the present invention.

### Compound 57: 2-{2-(4-Difluoromethoxyphenyl)-3-[2-(4-methoxyphenyl)ethyl]-1-methyl-4-oxo-1,3,8-triaza-spiro[4.5]dec-8-yl}acetamide

¹H-NMR (300 MHz, CDCl₃) δ 7.84 (b, 2H), 7.33 (d, 2H, J=8.5 Hz), 7.16 (d, 2H, J=8.8 Hz), 7.03 (d, 2H, J=8.4 Hz), 6.82 (d, 2H, J=8.5 Hz), 6.57, 6.33 (s, s, 1H), 4.56 (s, 1H), 4.09 (m, 1H), 3.99 (s, 2H), 3.83 (m, 1H), 3.81 (s, 3H), 3.60 (m, 2H), 3.39 (m, 1H), 2.77 (m, 2H), 2.55 (m, 1H), 2.33 (m, 2H), 2.07 (s, 3H), 1.94 (m, 1H), 1.35 (m, 1H); ¹³C-NMR (75 MHz, CDCl₃) δ 175.0; 163.0, 161.9, 158.9, 152.6, 133.6, 130.4, 130.2, 130.0, 122.2, 120.0, 116.0, 114.2, 112.0, 79.5, 77.8, 58.0, 55.6, 50.8, 50.4, 40.7, 32.8, 29.9, 24.1; MS MH⁺=503.2; elemental analysis: theory C₂₆H₃₂F₂N₄O₄+1.8 CF₃COOH C, 50.23; H, 4.81; N, 7.92. found C, 50.57; H, 5.00; N, 7.83.

Exemplary compounds of formula XX of the present invention can be prepared by the procedure outlined in Example 14 herein below. The skilled practitioner will know how to substitute the appropriate reagents, starting materials and purification methods known to those skilled in the art, in order to prepare the compounds provided herein.

### EXAMPLE 14

### Compound 58: 2-(4-tert-Butylbenzyl)-3-[2-(4-methoxyphenyl)ethyl]-1-methyl-4-oxo-1,3,8-triazaspiro[4.5]decane-8-carboxylic acid amide

Preparation of 2-(4-tert-butylbenzyl)-3-[2-(4-methyoxyphenyl)ethyl]-4-oxo-1,3,8-triaza-spiro[4.5]decane-8-carboxylic acid tert-butyl ester: To the solution of crude tert-butyl 4-((4-methoxyphenethyl)carbamoyl)-4-aminopiperidine-1-carboxylate, 2, (1.88 g, 5.0 mmol in 10 mL of methanol) and K₂CO₃ (1.38 g, 10.0 mmol) in a 10-20 mL Emry's process vial equipped with a stir bar is added 2-(4-tert-butylphenyl)acetaldehyde (885 mg, 5.0 mmol) via pipette. The reaction mixture is capped, stirred 30 seconds and heated in a Biotage Initiator 60 microwave for 25 minutes at 90° C. The reaction is cooled to room temperature, diluted with ethyl acetate (200 mL), washed with water (2×100 mL), dried over Na₂SO₄ and concentrated under reduced pressure to a crude residue which is purified over silica to afford 920 mg (34% yield) of the desired product. ¹H-NMR (300 MHz, CDCl₃) δ 7.37 (d, 2H, J=8.4), 7.11 (m, 4H), 6.86 (d, 2H, J=8.8 Hz), 4.55 (s, 1H), 4.02 (m, 1H), 3.95 (m, 2H), 3.81 (s, 3H), 3.24 (m, 1H), 3.18 (m, 2H), 3.10-2.75 (m, 4H), 1.84 (m, 1H), 1.66 (m, 1H), 1.48 (m, 1H), 1.42 (s, 9H), 1.32 (s, 9H), 1.30 (m, 1H), 1.02 (m, 1H); ¹³C-NMR (75 MHz, CDCl₃) δ 177.0; 158.7, 155.0, 132.2, 130.5, 129.7, 128.3, 126.0, 124.8, 120.5, 114.3, 79.9, 71.3, 59.7, 55.6, 42.1, 40.0, 39.8, 34.8, 34.2, 32.9, 31.7, 28.8; MS MH⁺=536.4; elemental analysis: theory C₃₂H₄₅N₃O₄+0.5H₂O C, 70.66; H, 8.51; N, 7.71. found C, 70.99; H, 8.29; N, 7.28.

Preparation of 2-(4-tert-butylbenzyl)-3-[2-(4-methyoxyphenyl)ethyl]-1-methyl-4-oxo-1,3,8-triaza-spiro[4.5]decane-8-carboxylic acid tert-butyl ester: To the solution of the 2-(4-tert-butylbenzyl)-3-[2-(4-methyoxyphenyl)ethyl]-4-oxo-1,3,8-triaza-spiro[4.5]decane-8-carboxylic acid tert-butyl ester (799.5 mg, 1.5 mmol in 10 mL of DMF) and CsCO₃ (648 mg, 2.0 mmol) in a 10-20 mL Emry's process vial equipped with a stir bar is added MeI (635 mg, 4.5 mmol) via pipet. The reaction mixture is then capped, stirred 30 sec. and heated in a Biotage Initiator 60 microwave for 40 minutes at 90° C. The reaction is then cooled to room temperature and diluted with EtOAc (150 mL), washed with water (2×50 mL). The combined organic extracts are then dried over anhydrous Na₂SO₄ and evaporated to dryness. The crude product is purified over silica to afford 560 mg (68% yield) of the desired product. ¹H-NMR (300 MHz, CDCl₃) δ 7.33 (d, 2H, J=8.5 Hz), 7.18 (d, 2H, J=8.7 Hz), 6.98 (d, 2H, J=8.8 Hz), 6.77 (d, 2H, J=8.9 Hz), 4.23 (s, 1H), 3.97 (m, 1H), 3.90 (m, 2H), 3.78 (s, 3H), 3.60 (m, 1H), 3.20 (m, 2H), 2.94 (m, 2H), 2.65 (m, 1H), 2.55 (m, 1H), 2.27 (s, 3H), 1.63 (m, 2H), 1.47 (s, 9H), 1.36 (, m, 1H), 1.33 (s, 9H), 1.04 (m, 1H); ¹³C-NMR (75 MHz, CDCl₃) δ 175.0; 158.7, 155.3, 150.0, 133.6, 130.4, 130.1, 129.7, 125.6, 114.1, 79.6, 76.9, 60.2, 55.6, 40.7, 40.2, 40.0, 38.1, 34.8, 32.9, 32.7, 31.7, 31.4, 28.8, 27.5; MS MH⁺=550.2; elemental analysis: theory C₃₃H₄₇N₃O₄ C, 72.10; H, 8.62; N, 7.64. found C, 72.02; H, 8.56; N, 7.29.

Preparation of 2-(4-tert-butylbenzyl)-3-[2-(4-methoxyphenyl)ethyl]-1-methyl-1,3,8-triazaspiro[4.5]decan-4-one: To a solution of the 2-(4-tert-butylbenzyl)-3-[2-(4-methyoxyphenyl)ethyl]-1-methyl-4-oxo-1,3,8-triaza-spiro[4.5]decane-8-carboxylic acid tert-butyl ester (494 mg, 0.9 mmol) in CH₂Cl₂ (30 mL) is added TFA (7.5 mL). After stirring at room temperature for 3 h, the aqueous NaHCO₃ (saturated, 100 mL) is added slowly and resulting mixture is stirred for 30 minutes at the room temperature. Two layers are separated and aqueous layer is extracted with CH₂Cl₂ (100 mL). The combined organic solvent is washed with aqueous NaHCO₃ and dried over NaSO₄. The solvent is removed in vacuo to give afford 435 mg (96% yield) of the desired product as a white solid. ¹H-NMR (300 MHz, CDCl₃) δ 7.34 (d, 2H, J=8.8 Hz), 7.16 (d, 2H, J=8.5 Hz), 7.02 (d, 2H, J=8.6 Hz), 6.80 (d, 2H, J=8.6 Hz), 4.24 (m, 1H), 4.06 (m, 1H), 3.79 (m, 1H), 3.78 (s, 3H), 3.22 (m, 1H), 3.13 (m, 3H), 3.06 (m, 2H), 2.73 (m, 1H), 2.57 (m, 1H), 2.32 (s, 3H), 1.92 (m, 2H), 1.84 (m, 1H), 1.33 (s, 9H), 1.04 (m, 1H); ¹³C-NMR (75 MHz, CDCl₃) δ 175.0; 158.7, 150.1, 133.2, 130.2, 130.1, 126.0, 114.1, 77.8, 58.8, 55.6, 41.5, 41.1, 40.5, 37.7, 34.8, 32.7, 31.7, 31.0, 30.9, 25.9; MS MH⁺=450.2; HRMS: theory C₂₈H₃₉N₃O₂ 450.3121; found 450.3114.

Preparation of 2-(4-tert-butylbenzyl)-3-[2-(4-methoxyphenyl)ethyl]-1-methyl-4-oxo-1,3,8-triazaspiro[4.5]decane-8-carboxylic acid amide: To a solution of the 2-(4-tert-butylbenzyl)-3-[2-(4-methoxyphenyl)ethyl]-1-methyl-1,3,8-triazaspiro[4.5]decan-4-one (328 mg, 0.85 mmol) in CH₂Cl₂ (30 mL) is added trimethylsilyl isocyanate (460 mg, 3.4 mmol), TEA (252 mg, 2.5 mmol). After stirring at room temperature for 6 h, the aqueous NaHCO₃ (saturated, 50 mL) is added and resulting mixture is stirred for 30 minutes at the room temperature. After CH₂Cl₂ (100 mL) is added and two layers are separated. The organic layer is washed with H₂O and dried over NaSO₄. The solvent is removed in vacuo to give crude product. The crude material obtained is purified over silica to afford 309 mg (74% yield) of the desired product. ¹H-NMR (300 MHz, CDCl₃) δ 7.34 (d, 2H, J=8.4 Hz), 7.18 (d, 2H, J=8.2 Hz), 6.99 (d, 2H, J=8.6 Hz), 6.80 (d, 2H, J=8.4 Hz), 4.52 (m, 2H), 4.26 (m, 1H), 4.02 (m, 1H), 3.81 (m, 2H), 3.79 (s, 3H), 3.61 (m, 1H), 3.23 (m, 1H), 3.15 (m, 1H), 2.90 (m, 2H), 2.70 (m, 1H), 2.58 (m, 1H), 2.27 (s, 3H), 1.65 (m, 2H), 1.57 (m, 1H), 1.35 (s, 9H), 1.10 (m, 1H); ¹³C-NMR (75 MHz, CDCl₃) δ 175.0; 158.7, 158.3, 150.0, 133.5, 130.3, 130.1, 129.7, 125.6, 114.1, 77.8, 60.5, 55.6, 41.6, 40.7, 40.4, 38.0, 34.8, 32.8, 32.7, 31.7, 31.4, 27.4; MS MH⁺=493.3; elemental analysis: theory C₂₉H₄₀N₄O₃ C, 70.70; H, 8.18; N, 11.37. found C, 70.35; H, 8.11; N, 11.16.

### EXAMPLE 15

Exemplary compounds of formula XXI of the present invention can be prepared by the procedure outlined in Example 15. The skilled practitioner will know how to substitute the appropriate reagents, starting materials and purification methods known to those skilled in the art, in order to prepare the compounds provided herein

### Compound 59: 8-(2-Amino-2-methylpropionyl)-2-(4-cyclopropylphenyl)-3-[2-(4-methoxyphenyl)ethyl]-1-methyl-1,3,8-triaza-spiro[4.5]decan-4-one

Preparation of (2-{2-(4-cyclopropylphenyl)-3-[2-(4-methoxyphenyl)ethyl]-1-methyl-4-oxo-1,3,8-triaza-spiro[4.5]dec-8-yl}-1,1-dimethyl-2-oxo-ethyl)-carbamic acid tert-butyl ester: To a solution of 2-(4-cyclopropylphenyl)-3-[2-(4-methoxyphenyl)-ethyl]-1-methyl-1,3,8-triaza-spiro[4.5]decan-4-one, 5, (476 mg contained 0.5 mmol TFA salt, 1.0 mmol) in CHCl₃ (20 mL) is added triethylamine (202 mg, 2 mmol) and 1-hydroxybenzotriazole (HOBt) (137 mg, 1 mmol). The resulting mixture is stirred for 10 minutes at room temperature and 2-(tert-butoxycarbonylamino) —2-methyl-propanoic acid (203 mg, 1 mmol) is added. The reaction mixture is stirred for 24 hours at room temperature. The reaction is washed with water and the aqueous layer extracted by CH₂Cl₂ (50 mL). The combined organic layers are washed with H₂O, dried over Na₂SO₄ and the solvent removed under reduced pressure and the resulting residue purified over silica to afford 460 mg (76% yield) of the desired product. ¹H-NMR (300 MHz, CDCl₃) δ 7.22 (d, 2H, J=7.9 Hz), 7.10 (d, 2H, J=7.9 Hz), 7.06 (d, 2H, J=8.2 Hz), 6.81 (d, 2H, J=8.3 Hz), 5.02 (b, 1H), 4.56 (s, 1H), 4.47 (b, 1H), 3.87 (m, 1H), 3.80 (s, 3H), 3.20 (m, 1H), 2.72 (m, 2H), 2.55 (m, 1H), 2.00 (s, 3H), 1.93 (m, 2H), 1.78 (m, 1H), 1.68 (m, 2H), 1.57 (m, 1H), 1.53 (s, 3H), 1.47 (s, 3H), 1.44 (s, 9H), 1.22 (m, 1H), 1.05 (m, 2H), 0.75 (m, 2H); ¹³C-NMR (75 MHz, CDCl₃) δ 176.0; 163.0, 158.7, 154.4, 146.1, 134.4, 130.5, 130.2, 128.9, 126.1, 114.1, 79.9, 60.4, 57.0, 55.7, 40.5, 33.0, 30.4, 28.7, 26.6, 25.6, 15.6, 10.0, 9.9; MS MH⁺=605.2; elemental analysis: theory C₃₅H₄₈N₄O₅+0.5H₂O C, 68.49; H, 8.05; N, 9.13. found C, 68.51; H, 8.04; N, 8.95.

Preparation of 8-(2-amino-2-methylpropionyl)-2-(4-cyclopropylphenyl) —3-[2-(4-methoxyphenyl)ethyl]-1-methyl-1,3,8-triaza-spiro[4.5]decan-4-one: To a solution of the (2-{2-(4-cyclopropylphenyl)-3-[2-(4-methoxyphenyl)ethyl]-1-methyl-4-oxo-1,3,8-triaza-spiro[4.5]dec-8-yl}-1,1-dimethyl-2-oxo-ethyl)-carbamic acid tert-butyl ester (320 mg, 0.5 mmol) in CH₂Cl₂ (10 mL) is added trifluoroacetic acid (2.5 mL). After stirring at room temperature for 2.5 hour, aqueous NaHCO₃ (saturated, 100 mL) is added slowly and resulting mixture is stirred for 30 minutes at the room temperature. The two layers which form are separated and the aqueous layer extracted with CH₂Cl₂ (100 mL). The organic layers are combined and washed with aqueous NaHCO₃, H₂O and dried over Na₂SO₄. The solvent is removed in vacuo to afford 204 mg (82% yield) of the desired product as a white solid. ¹H-NMR (300 MHz, CDCl₃) δ 7.22 (d, 2H, J=7.9 Hz), 7.09 (d, 2H, J=7.9 Hz), 7.06 (d, 2H, J=8.2 Hz), 6.83 (d, 2H, J=8.2 Hz), 4.52 (m, 3H), 4.00 (m, 1H), 3.89 (m, 2H), 3.79 (s, 3H), 3.20 (m, 1H), 2.75 (m, 2H), 2.52 (m, 1H), 2.02 (s, 3H), 1.94 (m, 1H), 1.89-1.56 (m, 4H), 1.42 (s, 6H), 1.26 (m, 1H), 1.02 (m, 2H), 0.73 (m, 2H); ¹³C-NMR (75 MHz, CDCl₃) δ 176.0; 175.0, 158.7, 146.1, 134.4, 130.5, 130.2, 128.9, 126.1, 114.1, 79.8, 60.4, 55.9, 55.6, 42.5, 41.3, 40.5, 33.2, 33.0, 30.4, 29.6, 26.8, 15.6, 10.0, 9.9; MS MH⁺=505.2; elemental analysis: theory C₃₀H₄₀N₄O₃+0.4 CF₃COOH C, 67.23; H, 7.40; N, 10.18. found C, 66.91; H, 7.56; N, 10.22.

Further compounds according to the present invention include:

### Compound 60: 2-(4-tert-Butylphenyl)-3-[2-(4-methoxyphenyl)ethyl]-4-oxo-1,3,8-triaza-spiro[4.5]decane-8-carboxylic acid tert-butyl ester

¹H-NMR (300 MHz, CDCl₃) δ 7.46 (d, 2H, J=8.4 Hz), 7.21 (d, 2H, J=8.3 Hz), 7.05 (d, 2H, J=8.8 Hz), 6.86 (d, 2H, J=8.7 Hz), 5.04 (s, 1H), 4.10 (m, 1H), 3.92 (m, 2H), 3.81 (s, 3H), 3.17 (m, 1H), 3.00 (m, 1H), 2.89-2.81 (m, 2H), 2.63 (m, 1H), 2.17 (m, 1H), 1.58 (m, 2H), 1.48 (s, 9H), 1.45 (m, 1H), 1.36 (s, 9H), 1.28 (m, 1H); ¹³C-NMR (75 MHz, CDCl₃) δ 177.0; 158.7, 155.0, 153.2, 135.5, 130.6, 130.2, 127.3, 126.5, 114.3, 79.9, 74.6, 60.5, 55.6, 41.9, 40.3, 35.1, 34.5, 34.4, 32.6, 31.8, 31.6, 28.8; MS MH⁺=522.5; elemental analysis: theory C₃₁H₄₃N₃O₄ C, 71.37; H, 8.31; N, 8.05. found C, 70.99; H, 7.91; N, 7.78.

### Compound 61: 2-(4-Diethylaminophenyl)-3-[2-(4-methoxyphenyl)ethyl]-4-oxo-1,3,8-triaza-spiro[4.5]decane-8-carboxylic acid tert-butyl ester

¹H-NMR (300 MHz, CDCl₃) δ 7.07 (m, 4H), 6.84 (d, 2H, J=8.4 Hz), 6.69 (d, 2H, J=8.8 Hz), 4.96 (s, 1H), 4.01 (m, 1H), 3.88 (m, 2H), 3.80 (s, 3H), 3.42 (q, 4H, J=6.9 Hz, J=14.1 Hz), 3.18 (m, 1H), 3.00 (m, 1H), 2.91 (m, 1H), 2.80 (m, 1H), 2.65 (m, 1H), 2.17 (m, 1H), 1.65 (m, 2H), 1.48 (m, 1H), 1.47 (s, 9H), 1.30 (m, 1H), 1.22 (t, 6H, J=6H); ¹³C-NMR (75 MHz, CDCl₃) δ 176.0, 158.6, 155.0, 149.0, 130.8, 128.7, 124.1, 114.2, 112.0, 79.9, 74.7, 60.4, 55.6, 51.2, 44.7, 41.8, 40.5, 39.7, 34.5, 32.6, 31.8, 28.8, 12.9; MS MH⁺=537.0.

### Compound 62: 2-(4-Difluoromethoxyphenyl)-3-[2-(4-methoxyphenyl)ethyl]-4-oxo-1,3,8-triaza-spiro[4.5]decane-8-carboxylic acid tert-butyl ester

¹H-NMR (300 MHz, CDCl₃) δ 7.28 (d, 2H, J=8.6 Hz), 7.25 (d, 2H, J=8.6 Hz), 7.04 (d, 2H, J=8.6 Hz), 6.85 (d, 2H, J=8.6 Hz), 6.56, 6.32 (s, s, 1H), 5.04 (s, 1H), 4.01 (m, 2H), 3.97 (m, 2H), 3.80 (s, 3H), 3.20 (m, 1H), 3.00 (m, 1H), 2.82 (m, 2H), 2.63 (m, 1H), 2.20 (m, 1H), 1.68 (m, 1H), 1.48 (m, 1H), 1.47 (s, 9H), 1.32 (m, 1H); ¹³C-NMR (75 MHz, CDCl₃) δ 176.0, 158.8, 154.9, 152.3, 130.4, 130.1, 129.3, 120.5, 119.4, 115.9, 114.4, 112.4, 80.0, 74.0, 60.5, 55.6, 42.0, 40.0, 39.8, 34.5, 32.6, 31.9, 28.8; MS MH⁺=532.0.

### Compound 63: 2-(4-tert-Butylphenyl)-8-methanesulfonyl-3-[2-(4-methoxyphenyl)ethyl]-1,3,8-triaza-spiro[4.5]decan-4-one

¹H-NMR (300 MHz, CDCl₃) δ 7.49 (d, 2H, J=8.3 Hz), 7.20 (d, 2H, J=8.7 Hz), 7.07 (d, 2H, J=8.4 Hz), 6.85 (d, 2H, J=8.2 Hz), 5.26 (s, 1H), 5.13 (bs, 1H), 4.04 (m, 1H), 3.81 (s, 3H), 3.74 (m, 1H), 3.49 (m, 1H), 3.28 (m, 2H), 2.88 (m, 2H), 2.80 (s, 3H), 2.67 (m, 1H), 2.31 (m, 1H), 1.85 (m, 2H), 1.74 (m, 1H), 1.37 (s, 9H); ¹³C-NMR (75 MHz, CDCl₃) δ 174.0; 159.0, 154.7, 131.1, 130.1, 129.6, 127.6, 126.8, 114.4, 73.3, 60.3, 55.6, 41.9, 35.5, 35.2, 33.1, 32.3, 31.5; MS MH⁺=500.1; elemental analysis: theory C₂₇H₃₇N₃O₄S+1.2 CF₃COOH C, 55.48; H, 6.05; N, 6.60. found C, 55.29; H, 5.85; N, 6.52.

### Compound 64: 2-(4-cyclopropylphenyl)-3-[2-(4-methoxyphenyl)ethyl]-4-oxo-1,3,8-triaza-spiro[4.5]decane-8-carboxylic acid tert-butyl ester

An alternative name for this compound is tert-butyl 2-(4-cyclopropylphenyl)-3-[2-(4-methoxyphenyl)ethyl]-4-oxo-1,3,8-triazaspiro[4.5]decane-8-carboxylate. ¹H-NMR (300 MHz, CDCl₃) δ 7.40 (m, 4H), 7.05 (d, 2H, J=8.8 Hz), 6.76 (d, 2H, J=8.7 Hz), 5.04 (s, 1H), 4.10 (m, 1H), 3.92 (m, 2H), 3.81 (s, 3H), 3.20 (m, 1H), 3.05 (m, 1H), 2.80 (m, 2H), 2.56 (m, 1H), 2.12 (m, 1H), 1.80 (m, 1H), 1.58 (m, 3H), 1.40 (s, 9H), 1.25 (m, 1H), 1.00 (m, 2H), 0.7 (m, 2H); ¹³C-NMR (75 MHz, CDCl₃) δ 177.0; 158.7, 155.0, 146.3, 135.5, 130.6, 130.1, 127.5, 126.7, 114.3, 79.9, 74.7, 60.4, 55.6, 41.8, 39.7, 39.4, 34.5, 32.6, 31.9, 28.8, 15.6, 10.0; MS MH⁺=506.2; elemental analysis: theory C₃₀H₃₉N₃O₄+0.1 CF₃COOH C, 70.15; H, 7.62; N, 8.13. found C, 70.32; H, 7.37; N, 8.11

### Compound 65: 2-(4-cyclopropylphenyl)-3-[2-(4-methoxyphenyl)ethyl]-1-methyl-4-oxo-1,3,8-triaza-spiro[4.5]decane-8-carboxylic acid tert-butyl ester:

An alternative name for this compound is tert-butyl 2-(4-cyclopropylphenyl)-3-[2-(4-methoxyphenyl)ethyl]-1-methyl-4-oxo-1,3,8-triazaspiro[4.5]decane-8-carboxylate. ¹H-NMR (300 MHz, CDCl₃) δ 7.23 (d, 2H, J=8.1 Hz), 7.10 (d, 2H, J=8.0 Hz), 7.05 (d, 2H, J=8.4 Hz), 6.84 (d, 2H, J=8.4 Hz), 4.52 (s, 1H), 4.10 (m, 1H), 3.90 (m, 2H), 3.80 (s, 3H), 3.20 (m, 1H), 3.05 (m, 1H), 2.76 (m, 2H), 2.50 (m, 1H), 2.03 (s, 3H), 1.93 (m, 1H), 1.58 (m, 3H), 1.40 (s, 9H), 1.15 (m, 1H), 1.01 (m, 2H), 0.7 (m, 2H); ¹³C-NMR (75 MHz, CDCl₃) δ 175.0; 158.7, 155.0, 146.1, 134.5, 130.4, 130.2, 128.9, 126.1, 114.1, 79.8, 79.7, 60.4, 55.6, 41.0, 40.6, 40.4, 32.9, 32.3, 30.4, 28.8, 15.6, 10.0; MS MH⁺=520.1; elemental analysis: theory C₃₁H₄₁N₃O₄ C, 71.65; H, 7.95; N, 8.09. found C, 71.98; H, 7.57; N, 7.83.

### Compound 66: 2-(4-methoxyphenyl)-3-[2-(4-methoxyphenyl)ethyl]-1-methyl-1,3,8-triaza-spiro[4.5]decan-4-one

¹H-NMR (300 MHz, CDCl₃) δ 7.29 (d, 2H, J=8.1 Hz), 7.06 (d, 2H, J=8.0 Hz), 6.94 (d, 2H, J=8.4 Hz), 6.82 (d, 2H, J=8.4 Hz), 4.55 (s, 1H), 3.85 (s, 2H), 3.80 (s, 3H), 3.09 (bs, 2H), 3.01 (m, 2H), 2.72 (m, 2H), 2.50 (m, 1H), 2.09 (s, 3H), 1.81 (m, 4H); MS MH⁺=410

### Compound 67: 8-(phenyl-N-cyano-1-carbimidate)-2-(4-methoxyphenyl)-3-[2-(4-methoxyphenyl)ethyl]-1-methyl-1,3,8-triaza-spiro[4.5]decan-4-one

An alternative name is phenyl N-cyano-3-(4-methoxyphenethyl)-2-(4-methoxyphenyl)-1-methyl-4-oxo-1,3,8-triazaspiro[4.5]decane-8-carbimidate. ¹H-NMR (300 MHz, CDCl₃) δ 7.45 (m, 2H), 7.29 (m, 3H), 7.11 (m, 4H), 6.95 (d, 2H, J=8.3 Hz), 6.83 (d, 2H, J=4.79 Hz), 4.56 (s, 1H), 4.16 (m, 2H), 3.86 (s, 3H), 3.79 (m, 1H), 3.64 (s, 3H), 3.46 (m, 1H), 2.75 (m, 2H), 2.55 (m, 1H), 2.08 (s, 3H), 1.85 (m, 3H), 1.29 (m, 2H); MH⁺=554.3; elemental analysis: theory C₃₂H₃₅N₅O₄+4.55 mol H₂O C, 60.46; H, 6.99; N, 11.01. found C, 60.46; H, 6.68; N, 10.89

### Compound 68: 2-(4-tert-butylbenzyl)-3-[2-(4-methyoxyphenyl)ethyl]-1-methyl-4-oxo-1,3,8-triaza-spiro[4.5]decane-8-carboxylic acid tert-butyl ester

An alternative name for this compound is tert-butyl 2-(4-tert-butylbenzyl)-3-(4-methoxyphenethyl)-1-methyl-4-oxo-1,3,8-triazaspiro[4.5]decane-8-carboxylate. ¹H-NMR (300 MHz, CDCl₃) δ 7.33 (d, 2H, J=8.5 Hz), 7.18 (d, 2H, J=8.7 Hz), 6.98 (d, 2H, J=8.8 Hz), 6.77 (d, 2H, J=8.9 Hz), 4.23 (s, 1H), 3.97 (m, 1H), 3.90 (m, 2H), 3.78 (s, 3H), 3.60 (m, 1H), 3.20 (m, 2H), 2.94 (m, 2H), 2.65 (m, 1H), 2.55 (m, 1H), 2.27 (s, 3H), 1.63 (m, 2H), 1.47 (s, 9H), 1.36 (, m, 1H), 1.33 (s, 9H), 1.04 (m, 1H); ¹³C-NMR (75 MHz, CDCl₃) δ 175.0; 158.7, 155.3, 150.0, 133.6, 130.4, 130.1, 129.7, 125.6, 114.1, 79.6, 76.9, 60.2, 55.6, 40.7, 40.2, 40.0, 38.1, 34.8, 32.9, 32.7, 31.7, 31.4, 28.8, 27.5; MS MH⁺=550.2; elemental analysis: theory C₃₃H₄₇N₃O₄ C, 72.10; H, 8.62; N, 7.64. found C, 72.02; H, 8.56; N, 7.29.

### Compound 69: 2-(4-tert-butylbenzyl)-3-[2-(4-methoxyphenyl)ethyl]-1-methyl-1,3,8-triazaspiro[4.5]decan-4-one

¹H-NMR (300 MHz, CDCl₃) δ 7.34 (d, 2H, J=8.8 Hz), 7.16 (d, 2H, J=8.5 Hz), 7.02 (d, 2H, J=8.6 Hz), 6.80 (d, 2H, J=8.6 Hz), 4.24 (m, 1H), 4.06 (m, 1H), 3.79 (m, 1H), 3.78 (s, 3H), 3.22 (m, 1H), 3.13 (m, 3H), 3.06 (m, 2H), 2.73 (m, 1H), 2.57 (m, 1H), 2.32 (s, 3H), 1.92 (m, 2H), 1.84 (m, 1H), 1.33 (s, 9H), 1.04 (m, 1H); ¹³C-NMR (75 MHz, CDCl₃) δ 175.0; 158.7, 150.1, 133.2, 130.2, 130.1, 126.0, 114.1, 77.8, 58.8, 55.6, 41.5, 41.1, 40.5, 37.7, 34.8, 32.7, 31.7, 31.0, 30.9, 25.9; MS MH⁺=450.2; HRMS: theory C₂₈H₃₉N₃O₂ 450.3121; found 450.3114

### Compound 70: (2-{2-(4-cyclopropylphenyl)-3-[2-(4-methoxyphenyl)ethyl]-1-methyl-4-oxo-1,3,8-triaza-spiro[4.5]dec-8-yl}-1,1-dimethyl-2-oxo-ethyl)-carbamic acid tert-butyl ester

¹H-NMR (300 MHz, CDCl₃) δ 7.22 (d, 2H, J=7.9 Hz), 7.10 (d, 2H, J=7.9 Hz), 7.06 (d, 2H, J=8.2 Hz), 6.81 (d, 2H, J=8.3 Hz), 5.02 (b, 1H), 4.56 (s, 1H), 4.47 (b, 1H), 3.87 (m, 1H), 3.80 (s, 3H), 3.20 (m, 1H), 2.72 (m, 2H), 2.55 (m, 1H), 2.00 (s, 3H), 1.93 (m, 2H), 1.78 (m, 1H), 1.68 (m, 2H), 1.57 (m, 1H), 1.53 (s, 3H), 1.47 (s, 3H), 1.44 (s, 9H), 1.22 (m, 1H), 1.05 (m, 2H), 0.75 (m, 2H); ¹³C-NMR (75 MHz, CDCl₃) δ 176.0; 163.0, 158.7, 154.4, 146.1, 134.4, 130.5, 130.2, 128.9, 126.1, 114.1, 79.9, 60.4, 57.0, 55.7, 40.5, 33.0, 30.4, 28.7, 26.6, 25.6, 15.6, 10.0, 9.9; MS MH⁺=605.2; elemental analysis: theory C₃₅H₄₈N₄O₅+0.5H₂O C, 68.49; H, 8.05; N, 9.13. found C, 68.51; H, 8.04; N, 8.95

### Compound 71: Phenyl N-cyano-2-(4-cyclopropylphenyl)-3-[2-(4-methoxyphenyl)ethyl]-1-methyl-4-oxo-1,3,8-triazaspiro[4.5]-decane-8-carboximidoate

### Compound 72: 2-(4-methoxyphenyl)-3-[2-(4-methoxyphenyl)ethyl]-1-methyl-4-oxo-1,3,8-triazaspiro[4.5]decane-8-carboxamide

HRMS: calcd for C₂₅H₃₂N₄O₄+ H+, 453.24963; found (ESI, [M+H]+Obs'd), 453.2489; HPLC Retention: 2.7 min.

### Compound 73: tert-butyl 2-(4-methoxyphenyl)-3-[2-(4-methoxyphenyl)ethyl]-4-oxo-1,3,8-triazaspiro[4.5]decane-8-carboxylate

HRMS: calcd for C₂₈H₃₇N₃O₅+H+, 496.28060; found (ESI, [M+H]+Obs'd), 496.2807; HPLC Retention: 3.1 min.

### Compound 74: 2-(4-cyclopropylphenyl)-3-[2-(4-methoxyphenyl)ethyl]-4-oxo-1,3,8-triazaspiro[4.5]decane-8-carboxamide

HRMS: calcd for C₂₆H₃₂N₄O₃+H+, 449.25472; found (ESI, [M+H]+Obs'd), 449.2550; HPLC Retention: 2.8 min.

### Compound 75: Methyl 2-(4-cyclopropylphenyl)-3-[2-(4-methoxyphenyl)ethyl]-4-oxo-1,3,8-triazaspiro[4.5]decane-8-carboxylate

HRMS: calcd for C₂₇H₃₃N₃O₄+H+, 464.25438; found (ESI, [M+H]+Obs'd), 464.2550; HPLC Retention: 3.0 min.

### Compound 76: 2-(4-tert-butylphenyl)-3-[2-(4-methoxyphenyl)ethyl]-1,3,8-triazaspiro[4.5]decan-4-one

HRMS: calcd for C₂₆H₃₅N₃O₂+H+, 422.28020; found (ESI, [M+H]+Obs'd), 422.2810; HPLC Retention: 2.9 min.

### Compound 77: tert-butyl 2-(4-cyclopropylphenyl)-1-methyl-4-oxo-3-{2-[4-(trifluoromethoxy)phenyl]ethyl}-1,3,8-triazaspiro[4.5]decane-8-carboxylate

HRMS: calcd for C₃₁H₃₈F₃N₃O₄+H+, 574.28872; found (ESI, [M+H]+Obs'd), 574.2887; HPLC Retention: 3.5 min.

### Compound 78: 2-(4-cyclopropylphenyl)-1-methyl-3-{2-[4-(trifluoromethoxy)phenyl]ethyl}-1,3,8-triazaspiro[4.5]decan-4-one

HRMS: calcd for C₂₆H₃₀F₃N₃O₂+H+, 474.23629; found (ESI, [M+H]+Obs'd), 474.2371; HPLC Retention: 3.2 min.

### Compound 79: 2-(4-cyclopropylphenyl)-3-[2-(4-methoxyphenyl)ethyl]-8-(methylsulfonyl)-1,3,8-triazaspiro[4.5]decan-4-one

HRMS: calcd for C₂₆H₃₃N₃O₄S+H+, 484.22645; found (ESI, [M+H]+Obs'd), 484.2270; HPLC Retention: 3.0 min.

### Compound 80: 2-(4-cyclopropylphenyl)-3-[2-(4-methoxyphenyl)ethyl]-1-methyl-4-oxo-1,3,8-triazaspiro[4.5]decane-8-carbohydrazide

HRMS: calcd for C₂₇H₃₅N₅O₃+H+, 478.28127; found (ESI, [M+H]+Obs'd), 478.2814; HPLC Retention: 2.9 min.

### Compound 81: 2-[4-(difluoromethoxy)phenyl]-3-[2-(4-methoxyphenyl)ethyl]-1-methyl-8-pentanoyl-1,3,8-triazaspiro[4.5]decan-4-one

HRMS: calcd for C₂₉H₃₇F₂N₃O₄+H+, 530.28249; found (ESI, [M+H]+Obs'd), 530.2828; HPLC

Retention: 3.2 min.

### Compound 82: 8-(cyclopentylcarbonyl)-2-[4-(difluoromethoxy)phenyl]-3-[2-(4-methoxyphenyl)ethyl]-1-methyl-1,3,8-triazaspiro[4.5]decan-4-one

HRMS: calcd for C₃₀H₃₇F₂N₃O₄+H+, 542.28249; found (ESI, [M+H]+Obs'd), 542.2829; HPLC

Retention: 3.2 min.

### Compound 83: 8-(cyclopropylcarbonyl)-2-(4-isobutylphenyl)-3-[2-(4-methoxyphenyl)ethyl]-1-methyl-1,3,8-triazaspiro[4.5]decan-4-one;

HRMS: calcd for C₃₁H₄₁N₃O₃+H+, 504.32207; found (ESI, [M+H]+Obs'd), 504.3226; HPLC Retention: 3.4 min.

### Compound 84: 2-(4-cyclopropylphenyl)-3-[2-(4-methoxyphenyl)ethyl]-1-methyl-4-oxo-1,3,8-triazaspiro[4.5]decane-8-carboximidamide

HRMS: calcd for C₂₇H₃₅N₅O₂+H+, 462.28635; found (ESI, [M+H]+Obs'd), 462.2867; HPLC Retention: 3.0 min.

### Compound 85: tert-butyl 2-(4-isobutylphenyl)-3-[2-(4-methoxyphenyl)ethyl]-1-methyl-4-oxo-1,3,8-triazaspiro[4.5]decane-8-carboxylate

HRMS: calcd for C₃₂H₄₅N₃O₄+H+, 536.34828; found (ESI, [M+H]+Obs'd), 536.3487; HPLC Retention: 3.7 min.

### Compound 86: 2-(4-cyclopropylphenyl)-3-[2-(4-methoxyphenyl)ethyl]-N′,1-dimethyl-4-oxo-1,3,8-triazaspiro[4.5]decane-8-carboximidamide

HRMS: calcd for C₂₈H₃₇N₅O₂+H+, 476.30200; found (ESI, [M+H]+Obs'd), 476.3027; HPLC Retention: 3.0 min.

### Compound 87: tert-butyl 2-(4-methoxyphenyl)-4-oxo-3-(3-phenylpropyl)-1,3,8-triazaspiro[4.5]decane-8-carboxylate

HRMS: calcd for C₂₈H₃₇N₃O₄+H+, 480.28568; found (ESI, [M+H]+Obs'd), 480.2863; HPLC Retention: 3.1 min.

### Compound 88: 2-(4-tert-butylphenyl)-8-(cyclopropylcarbonyl)-3-[2-(4-methoxyphenyl)ethyl]-1,3,8-triazaspiro[4.5]decan-4-one

HRMS: calcd for C₃₀H₃₉N₃O₃+H+, 490.30642; found (ESI, [M+H]+Obs'd), 490.3071; HPLC Retention:3.1 min.

### Compound 89: 2-{2-(4-cyclopropylphenyl)-3-[2-(4-methoxyphenyl)ethyl]-1-methyl-4-oxo-1,3,8-triazaspiro[4.5]dec-8-yl}-N,N-dimethylacetamide

HRMS: calcd for C₃₀H₄₀N₄O₃+H+, 505.31732; found (ESI, [M+H]+Obs'd), 505.3180; HPLC Retention: 3.0 min.

### Compound 90: 2-(4-cyclopropylphenyl)-3-[2-(4-methoxyphenyl)ethyl]-1-methyl-8-D-prolyl-1,3,8-triazaspiro[4.5]decan-4-one

HRMS: calcd for C₃₁H₄₀N₄O₃+H+, 517.31732; found (ESI, [M+H]+Obs'd), 517.3176; HPLC Retention: 3.0 min.

### Compound 91: 2-(4-cyclopropylphenyl)-8-(1H-imidazol-1-ylcarbonyl) —3-[2-(4-methoxyphenyl)ethyl]-1-methyl-1,3,8-triazaspiro[4.5]decan-4-one

HRMS: calcd for C₃₀H₃₅N₅O₃+H+, 514.28127; found (ESI, [M+H]+Obs'd), 514.2813; HPLC Retention: 3.1 min.

### Compound 92: 2-(4-cyclopropylphenyl)-3-[2-(4-methoxyphenyl)ethyl]-8-(methylsulfonyl)-1-(trifluoroacetyl)-1,3,8-triazaspiro[4.5]decan-4-one

HRMS: calcd for C₂₈H₃₂F₃N₃O₅S+H+, 580.20875; found (ESI, [M+H]+Obs'd), 580.2090; HPLC Retention: 3.2 min.

### Compound 93: 2-(4-cyclopropylphenyl)-3-[2-(4-methoxyphenyl)ethyl]-1,3,8-triazaspiro[4.5]decan-4-one

HRMS: calcd for C₂₅H₃₁N₃O₂+H+, 406.24890; found (ESI, [M+H]+Obs'd), 406.2494; HPLC Retention: 2.6 min.

### Compound 94: 2-(4-tert-butylphenyl)-8-(ethylsulfonyl)-3-[2-(4-methoxyphenyl)ethyl]-1,3,8-triazaspiro[4.5]decan-4-one

HRMS: calcd for C₂₈H₃₉N₃O₄S+H+, 514.27340; found (ESI, [M+H]+Obs'd), 514.2740; Retention: 3.2

### Compound 95: 1-methyl-2-(4-trifluoromethylphenyl)-3-[2-(4-methoxyphenyl)-ethyl]-4-oxo-1,3,8-triaza-spiro[4.5]decane-8-carboxylic acid tert-butyl ester

### Compound 96: 2-(4-tert-butylbenzyl)-3-[2-(4-methyoxyphenyl)ethyl]-4-oxo-1,3,8-triaza-spiro[4.5]decane-8-carboxylic acid tert-butyl ester

An alternative name for this compound is tert-butyl 2-(4-tert-butylbenzyl)-3-(4-methoxyphenethyl)-4-oxo-1,3,8-triazaspiro[4.5]decane-8-carboxylate. ¹H-NMR (300 MHz, CDCl₃) δ 7.37 (d, 2H, J=8.4), 7.11 (m, 4H), 6.86 (d, 2H, J=8.8 Hz), 4.55 (s, 1H), 4.02 (m, 1H), 3.95 (m, 2H), 3.81 (s, 3H), 3.24 (m, 1H), 3.18 (m, 2H), 3.10-2.75 (m, 4H), 1.84 (m, 1H), 1.66 (m, 1H), 1.48 (m, 1H), 1.42 (s, 9H), 1.32 (s, 9H), 1.30 (m, 1H), 1.02 (m, 1H); ¹³C-NMR (75 MHz, CDCl₃) δ 177.0; 158.7, 155.0, 132.2, 130.5, 129.7, 128.3, 126.0, 124.8, 120.5, 114.3, 79.9, 71.3, 59.7, 55.6, 42.1, 40.0, 39.8, 34.8, 34.2, 32.9, 31.7, 28.8; MS MH⁺=536.4; elemental analysis: theory C₃₂H₄₅N₃O₄+0.5H₂O C, 70.66; H, 8.51; N, 7.71. found C, 70.99; H, 8.29; N, 7.28.

### Compound 97: tert-butyl 2-(4-tert-butylbenzyl)-3-[2-(4-methoxyphenyl)ethyl]-1-methyl-4-oxo-1,3,8-triazaspiro[4.5]decane-8-carboxylate

¹H-NMR (300 MHz, CDCl₃) δ 7.33 (d, 2H, J=8.5 Hz), 7.18 (d, 2H, J=8.7 Hz), 6.98 (d, 2H, J=8.8 Hz), 6.77 (d, 2H, J=8.9 Hz), 4.23 (s, 1H), 3.97 (m, 1H), 3.90 (m, 2H), 3.78 (s, 3H), 3.60 (m, 1H), 3.20 (m, 2H), 2.94 (m, 2H), 2.65 (m, 1H), 2.55 (m, 1H), 2.27 (s, 3H), 1.63 (m, 2H), 1.47 (s, 9H), 1.36 (, m, 1H), 1.33 (s, 9H), 1.04 (m, 1H); ¹³C-NMR (75 MHz, CDCl₃) δ 175.0; 158.7, 155.3, 150.0, 133.6, 130.4, 130.1, 129.7, 125.6, 114.1, 79.6, 76.9, 60.2, 55.6, 40.7, 40.2, 40.0, 38.1, 34.8, 32.9, 32.7, 31.7, 31.4, 28.8, 27.5; MS MH⁺=550.2; elemental analysis: theory C₃₃H₄₇N₃O₄ C, 72.10; H, 8.62; N, 7.64. found C, 72.02; H, 8.56; N, 7.29.

### Compound 98: (S)-2-(4-cyclopropylphenyl)-3-[2-(4-methoxyphenyl)ethyl]-1-methyl-4-oxo-1,3,8-triaza-spiro[4.5]decane-8-carboxylic acid amide

¹H-NMR (300 MHz, CDCl₃) δ 7.20 (d, 2H, J=8.2 Hz), 7.10 (d, 2H, J=8.2 Hz), 7.03 (d, 2H, J=8.6 Hz), 6.84 (d, 2H, J=8.7 Hz), 4.60 (b, 2H), 4.54 (s, 1H), 3.99 (m, 1H), 3.89 (m, 3H), 3.80 (s, 3H), 3.23 (m, 1H), 2.76 (m, 2H), 2.51 (m, 1H), 2.04 (s, 3H), 1.93 (m, 1H), 1.76 (m, 3H), 1.21 (m, 1H), 1.03 (m, 2H), 0.74 (m, 2H); ¹³C-NMR (75 MHz, CDCl₃) δ 175.0; 158.7, 158.4, 146.1, 134.4, 130.5, 130.2, 128.9, 126.2, 114.1, 79.8, 60.3, 55.7, 41.5, 40.5, 40.4, 32.9, 32.6, 30.4, 26.3, 15.6, 10.0, 9.9; MS MH⁺=463.3; elemental analysis: theory C₂₇H₃₄N₄O₃ C, 70.10; H, 7.41; N, 12.11. found C, 70.07; H, 7.47; N, 12.09.

### Compound 99: (R)-2-(4-cyclopropylphenyl)-3-[2-(4-methoxyphenyl)ethyl]-1-methyl-4-oxo-1,3,8-triaza-spiro[4.5]decane-8-carboxylic acid amide

¹H-NMR (300 MHz, CDCl₃) δ 7.20 (d, 2H, J=8.2 Hz), 7.10 (d, 2H, J=8.2 Hz), 7.03 (d, 2H, J=8.6 Hz), 6.84 (d, 2H, J=8.7 Hz), 4.60 (b, 2H), 4.54 (s, 1H), 3.99 (m, 1H), 3.89 (m, 3H), 3.80 (s, 3H), 3.23 (m, 1H), 2.76 (m, 2H), 2.51 (m, 1H), 2.04 (s, 3H), 1.93 (m, 1H), 1.76 (m, 3H), 1.21 (m, 1H), 1.03 (m, 2H), 0.74 (m, 2H); ¹³C-NMR (75 MHz, CDCl₃) δ 175.0; 158.7, 158.4, 146.1, 134.4, 130.5, 130.2, 128.9, 126.2, 114.1, 79.8, 60.3, 55.7, 41.5, 40.5, 40.4, 32.9, 32.6, 30.4, 26.3, 15.6, 10.0, 9.9; MS MH⁺=463.3; elemental analysis: theory C₂₇H₃₄N₄O₃ C, 70.10; H, 7.41; N, 12.11. found C, 70.07; H, 7.47; N, 12.09.

### Compound 100: 2-(4-tert-Butylphenyl)-1-methyl-4-oxo-3-(3-phenylpropyl)-1,3,8-triazaspiro-[4.5]decane-8-carboxylic acid tert-butyl ester

### Compound 101: 2-(4-Cyclopropylphenyl)-N-ethoxy-3-[2-(4-methoxyphenyl)ethyl]-N,1-dimethyl-4-oxo-1,3,8-triazaspiro[4.5]decane-8-carboxamide

HPLC conditions for compounds 72-94 were as follows: Column: BDS

Hypersil C8; mobile phase A: 10 mM NH₄OAC in 95% water/5% ACN (pipette 6.67 mL of 7.5 M NH₄OAC solution into 4743 mL H₂O, then add 250 mL of ACN to the solution and mix.); mobile phase B: 10 mM NH₄OAC in 5% water/95% ACN (pipette 6.67 mL of 7.5 M NH₄OAC solution into 243 mL H₂O. Then add 4750 mL of ACN to the solution and mix.); flow Rate: 0.800 mL/min; column Temperature: 40° C.; injection Volume: 5 [symbol] L; UV: monitor 214 nm and 254 nm; gradient table (time (min)/% B): 0.0/0; 2.5/100; 4.0/100; 4.1/0; 5.5/0.

### Process

The present invention further relates to a process for preparing the Kv1.5 potassium channel inhibitors of the present invention.

Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatograpy (HPLC), gas chromatography (GC), gel-permeation chromatography (GPC), or thin layer chromatography (TLC).

Preparation of the Compounds can Involve Protection and Deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The first aspect of the process of the present invention relates to a process for preparing 5-spirocyclic-4-imidazolidinone Kv1.5 potassium channel inhibitors having the formula: 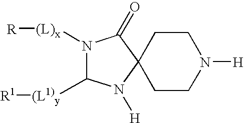
wherein R is optionally substituted phenyl;
R¹ is optionally substituted phenyl;
L and L¹ are linking units each independently a unit having the formula:
—[C(R¹⁹)₂]ₙ—
each R¹⁹ is independently chosen from hydrogen, methyl, or ethyl;
n is 1 to 4; and
x and y are each independently 0 or 1.

The 5-spirocyclic-4-imidazolidinone formed in this synthesis also can serve as an intermediate for preparing Kv1.5 potassium channel inhibitors of the present invention having formula (I).

The first aspect of the process of the present invention comprises the steps of:
a) reacting an amine having the formula:
   R-(L)ₓ-NH₂
   with an intermediate having the formula: 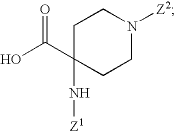
   or an intermediate having the formula: 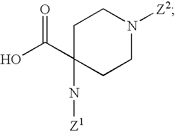
   wherein Z¹ and Z² are nitrogen protecting groups such that Z¹ and Z² are each removable by a means which does not affect and/or remove the other protecting group and Z¹ is capable of forming one or two bonds with nitrogen; to form an amide having the formula: 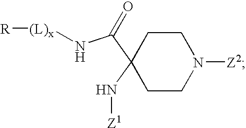
   or the formula: 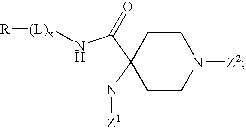
b) removing the protecting group Z¹ to form a mono-protected spirocyclic precursor amine having the formula: 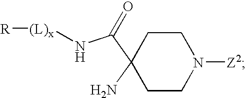
c) reacting the mono-protected spirocyclic precursor amine formed in step (b) with an aldehyde having the formula:
   R¹-(L¹)_{y}-CHO
   to form a protected 5-spirocyclic-4-imidazolidinone having the formula: 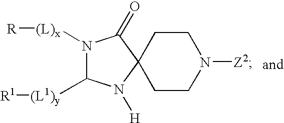
d) removing the nitrogen protecting group Z² to form the 5-spirocyclic-4-imidazolidinone Kv1.5 potassium channel inhibitor having the formula:

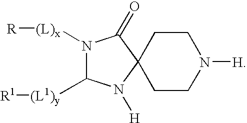

The first step in the process of the present invention, step (a), relates to reacting an amine with a protected intermediate having the formula: 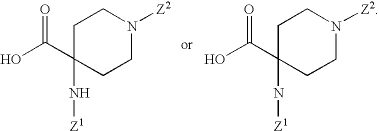
Z¹ and Z² should each be removable by a means which does not affect and/or remove the other protecting group, that is, Z¹ and Z² should be capable of sequential removal. The process for removing Z¹ should not effect Z² and vice versa. Z¹ is a protecting group which may form either one or two bonds with the primary amino unit of the intermediate. Examples of single bond protecting groups include benzyloxycarbonyl (Cbz), tert-butoxycarbonyl (Boc), [(9H-fluoren-9-yl)methoxy]carbonyl (Fmoc), and the like. Examples of two bond protecting groups includes phthalimido. Any suitable single bond protecting group can serve as Z² provided the means for removing Z¹ does not also remove Z² or vice versa. The chemistry of protecting groups can be found, for example, in Greene et al., Protective Groups in Organic Synthesis, 2d. Ed. (Wiley & Sons, 1991).

Step (a) can be conducted in the presence of a solvent, non-limiting examples of which include dimethylformamide (DMF), dichloromethane (CH₂Cl₂), 1,1-dichloroethane (CHCl₂CH₃), dimethylsulfoxide (DMSO), ethyl acetate (EtOAc), and the like.

A catalyst may be used to activate the intermediate carboxylic acid towards reaction with the amine. Non-limiting examples of suitable catalysts include benzotriazole-2-yl-(oxy-tris-pyrrolidino)-phosphonium hexafluorophosphate (PyBOP), O-(7-azabenzotriazol-1-yl)-N,N,N′,N′-tetramethyluronium hexafluorophosphate (HATU), 1-(3-dimethylaminopropyl)-3-ethylcarbodiimide (EDCI), dicyclodhexylcarbodiimide (DCC), and the like.

In addition to an optional catalyst, an organic or inorganic base can be utilized to assist in ensuring the reactivity of the amine. Non-limiting examples of organic bases include: triethylamine (TEA), diisopropylamine (DIPA), diisopropylethylamine (DIPEA), N-methylmorpholine (NMM), pyridine, and s-lutidine. The time and temperature of the reaction can be adjusted by the formulator to achieve optimal yields. These adjustments are within the scope of ordinary conditions which are familiar to the artisan of skill.

The second step of the process of the present invention relates to the selective removal of the protecting group Z¹. This is accomplished in a manner which leaves the Z² protecting group intact. This differential removal of Z¹ can be accomplished by selecting the proper protecting group in the previous steps or purchasing commercially available compounds for use in the present process. This step can be carried out under any conditions which do not change or modify the core structure of the molecule and which leaves the protecting group Z² intact. A non-limiting example of a group which is removed in this step is 9-fluorenylmethyl carbamate “Fmoc” which can removed by heating the intermediate formed in Step (a) in DMF, glyme, diglyme, dioxane, or other high boiling solvent with a catalytic amount of an organic or an inorganic base, non-limiting examples of which include piperidine, morpholine, ethanolamine, sodium carbonate, sodium bicarbonate, and the like. Therefore, a protecting group such as “Fmoc” which is removable with base is compatible with Z² protecting groups which can be removed by acid cleavage, for example, tert-butoxycarbonyl (Boc), or hydrogenolysis, for example, Carbobenzyloxy (Cbz).

The third step of the process of the present invention relates to the reaction of an aldehyde having the formula:
R¹-(L¹)_{y}-CHO
with a compound formed in step (b) wherein the Z¹ protecting group has been removed to form a protected 5-spirocyclic-4-imidazolidinone having the formula:

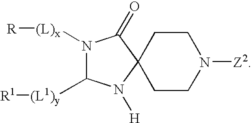

In one embodiment, microwave radiation is used to heat the reaction in step (c). The reaction, if conducted in the presence of a solvent, will comprise sufficient solvent to insure complete solution of the reactants. Non-limiting examples of solvents suitable for use include: C₁-C₆ linear, branched, or cyclic alcohols, inter alia, methanol, ethanol, iso-propanol, and the like; esters, inter alia, methyl acetate, ethyl acetate, and the like; halogenated C₁-C₂ alkanes, inter alia, methylene chloride, chloroform, carbon tetrachloride, 1,2 dichloroethane, 1,1-dichloroethane, 1,1,1-trichloroethane, and the like; ethers, inter alia, tetrahydrofuran, diethylether, methyl tert-butyl ether, and the like.

In addition to the optional presence of a solvent, an organic or inorganic base can also be used to further the rate of reaction. Non-limiting examples of inorganic bases includes NaHCO₃, Na₂CO₃, K₂CO₃, and the like.

As it relates to the final compounds of the present invention, in the case wherein Z² serves as a protecting group, as well as a suitable R³ unit, the product of step (c) will result in a Kv1.5 potassium channel inhibitor according to the present invention. For example, if Z² is a —SO₂CH₃ unit, this will serve the purpose of protecting the ring nitrogen from reaction and this unit is a R³ as described herein above and claimed herein below.

The fourth step of the process of the present invention relates to removal of the Z² protecting group. This step produces compounds wherein R³ is hydrogen. Compounds wherein R³ is hydrogen are both Kv1.5 potassium channel inhibitors, as well as intermediates for analogs wherein R³ comprises a moiety defined herein above. The conditions under which the R³ group is introduced is dependent upon the structure of the moiety being introduced and the reactivity of the reagent which introduces said moiety.

In one embodiment step (d) is followed by step (e):
e) reacting said inhibitor formed in step (d) having the formula: 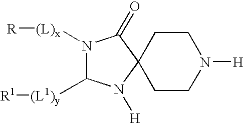
   with a reagent chosen from, for example:
   i) R¹⁶SO₂Cl;
   ii) R⁴C(O)Cl;
   iii) BrCH₂CONH₂;
   iv) (R⁵)NCO;
   v) (R⁵R⁶)NC(O)Cl; and
   v) (CH₃)₃SiNCO;
   to form a Kv1.5 potassium channel inhibitor having the formula: 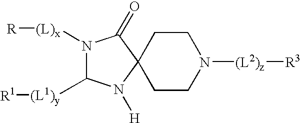
   as described herein above.

In some embodiments, the 1-position ring nitrogen (R² unit) is alkylated prior to removal of the Z² protecting group (step (d) above). This embodiment includes:
d) (ii) reacting the protected 5-spirocyclic-4-imidazolidinone formed in step (c) above having the formula: 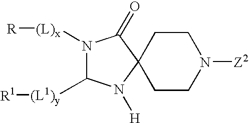
   with an alkylating agent to form an N-alkyl-5-spirocyclic-4-imidazolidinone having the formula: 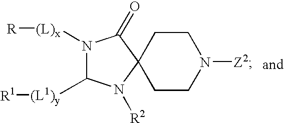
e)(ii) removing the nitrogen protecting group Z² from the N-alkyl-5-spirocyclic-4-imidazolidinone formed in step (d)(ii) to form the 5-spirocyclic-4-imidazolidinone Kv1.5 potassium channel inhibitor having the formula:

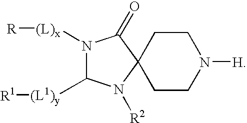

Step (d)(ii) utilizes an alkylating agent to introduce R² when R² is C₁-C₆ linear or branched alkyl (e.g., methyl, ethyl, propyl, or isopropyl). Any alkylating agent is suitable for use, for example, methyl iodide, ethyl iodide, and the like. The reaction can be conducted in the presence of a solvent, in one iteration the solvent is a polar aprotic solvent, inter alia, dimethylformamide (DMF), dimethylsulfoxide (DMSO), tetrahydrofuran (THF), and the like. A non-nucleophilic organic or inorganic base may be used to activate the compound formed in step (d) toward displacement of the alkylating agent's leaving group. In one embodiment, CsCO₃ is used. The reaction can be conducted at any temperature which the artisan finds suitable and adaptable to the relative reactivities of the reagents at hand. In one embodiment, the reaction is conducted in a microwave reactor, however, the formulator may vary the time and temperature which is necessary without undue experimentation.

The fifth step of the process of the present invention relates to removal of the Z² protecting group. This step produces compounds wherein R³ is hydrogen. Compounds wherein R³ is hydrogen are both Kv1.5 potassium channel inhibitors, well as intermediates for analogs wherein R³ comprises a moiety defined herein above. The conditions under which the R³ group is introduced is dependent upon the structure of the moiety being introduced and the reactivity of the reagent which introduces said moiety.

In one embodiment step (e)(ii) is followed by step (f)(ii):
f) (ii) reacting said inhibitor formed in step (e)(ii) having the formula: 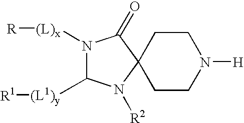
   with a reagent chosen from, for example:
   i) R¹⁶SO₂Cl;
   ii) R⁴C(O)Cl;
   iii) BrCH₂CONH₂;
   iv) (R⁵)NCO;
   v) (R⁵R⁶)NC(O)Cl; and
   v) (CH₃)₃SiNCO;
      to form a Kv1.5 potassium channel inhibitor having the formula: 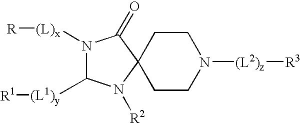
      as described herein above.

### Enantiomer Separation

The present invention provides enantiomerically pure R and S enantiomers of the compounds provided herein. Methods of resolving enantiomers are known in the art. For example, a supercritical fluid chromatography (SFC) method can be used to resolve the enantiomers. For example, using a SFC method, compound 7 was resolved into (S)-2-(4-cyclopropylphenyl)-3-[2-(4-methoxyphenyl)ethyl]-1-methyl-4-oxo-1,3,8-triaza-spiro[4.5]decane-8-carboxylic acid amide and (R)-2-(4-cyclopropylphenyl) —3-[2-(4-methoxyphenyl)ethyl]-1-methyl-4-oxo-1,3,8-triaza-spiro[4.5]decane-8-carboxylic acid amide. 317 mg of Compound 7 (approximately 52% purity) was chromatographed on a Kromasil CN 20×250 mm column using 20% MeOH (0.2% dimethylethylamine) 80% CO₂ (317 mg in 8 ml, 8 injections) to provide a pure compound. The material was immediately chirally resolved on a Chiralcel OJ-H 20×250 mm column using 35% MeOH 65% CO₂ to provide the two enantiomers (S)-2-(4-cyclopropylphenyl)-3-[2-(4-methoxyphenyl)ethyl]-1-methyl-4-oxo-1,3,8-triaza-spiro[4.5]decane-8-carboxylic acid amide (100 mg, retention time 2.95 min) and (R)-2-(4-cyclopropylphenyl)-3-[2-(4-methoxyphenyl)ethyl]-1-methyl-4-oxo-1,3,8-triaza-spiro[4.5]decane-8-carboxylic acid amide (96 mg, retention time 5.88 min).

Compounds listed and described herein have been found in many instances to exhibit activities (IC₅₀) in the assays described or referenced herein at concentrations below 1 micromolar (μM).

Compounds of the present invention are effective as Kv1.5 potassium channel inhibitors. Accordingly, compounds of the present invention can be used to prevent or treat conditions that can be affected by inhibition of Kv1.5 potassium channel.

Compounds of the present invention can be used to treat or prevent cardiac arrhythmias, including atrial fibrillation and flutter. In preferred embodiments, compounds of the present invention are capable of inhibiting Kv1.5 potassium channels while having little or no inhibitory effect on other ion channels in heart, including for example, ion channels in the ventricles. Accordingly, in particularly preferred embodiments, compounds of the present invention will prevent or treat cardiac arrhythmia while avoiding some of the common complications typically associated with inhibition of ion channels in the heart, including, for example, a prolongation of the QT interval and an increased propensity for life threatening ventricular arrhythmias.

Compounds of the present invention can be used to treat or prevent atrial arrhythmias, including atrial fibrillation and atrial flutter, as well as conditions associated with atrial arrhythmias, including, for example, thromboembolism, stroke, and heart failure.

Compounds of the present invention can be used to produce long-term, as well as short term maintenance periods free of arrhythmia in patients with persistent or chronic atrial arrhythmias.

Compounds of the present invention can also be used to prophylacticly treat post surgical atrial arrhthmias.

Methods of the present invention thus include methods of inhibiting Kv1.5 potassium channel; methods of inhibiting Kv1.5 potassium channels while having little or no inhibitory effect on other ion channels in heart, including for example, ion channels in the ventricles; methods of treating or preventing cardiac arrhythmias, including atrial fibrillation and flutter; methods for treating or preventing conditions associated with atrial arrhythmias, including, for example, thromboembolism, stroke, and heart failure; methods for producing long-term, as well as short term maintenance periods free of arrhythmia in patients with persistent or chronic atrial arrhythmias; and methods for prophylacticly treating post surgical atrial arrhthmias. The methods can comprise administering an effective amount of a compound or composition of the present invention to a subject.

The present invention also relates to the use of the 5-spirocyclic-4-imidazolidinones according to the present invention in the manufacture of a medicament for the treatment or prevention of atrial arrhythmias and related disorders.

The present invention further relates to forms of the present compounds, which under normal human or higher mammalian physiological conditions, release the compounds described herein. This aspect includes the pharmaceutically acceptable salts of the analogs described herein. The formulator, for the purposes of compatibility with delivery mode, excipients, and the like, can select one salt form of the present analogs over another since the compounds themselves are the active species which mitigate the disease processes described herein.

### Formulations

The present invention also relates to compositions or formulations which comprise the Kv1.5 potassium channel inhibitors according to the present invention. In general, the compositions of the present invention comprise an effective amount of one or more 5-spirocyclic-4-imidazolidinones and salts thereof according to the present invention which are effective for providing atrial-selective antiarrhythmia; and one or more excipients.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known antiarrhythmic agents. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present teachings can be useful for the treatment or inhibition of a pathological condition or disorder in a mammal, for example, a human subject. The present teachings accordingly provide methods of treating or inhibiting a pathological condition or disorder by providing to a mammal a compound of the present teachings including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Non-limiting examples of compositions according to the present invention include from about 0.001 mg to about 1000 mg of one or more 5-spirocyclic-4-imidazolidinones according to the present invention and one or more excipients; from about 0.01 mg to about 100 mg of one or more 5-spirocyclic-4-imidazolidinones according to the present invention and one or more excipients; and from about 0.1 mg to about 10 mg of one or more 5-spirocyclic-4-imidazolidinones according to the present invention; and one or more excipients.

### Procedures

The following procedures can be utilized in evaluating and selecting compounds as the Kv1.5 potassium channel inhibitors.

### FLIPR L-type Calcium Channel Assay^{1,2}

HL-1 cells expressing endogenous L-type calcium channels are removed from culture flasks using trypsin, plated on fibronectin/gelatin-coated, clear-bottomed, black-walled 96-well microplates in Claycomb media (JRH Biosciences #51800) containing 10% fetal bovine serum, 4 mM L-glutamine, and 10 μM norepinephrine, and grown to confluency overnight. The next day, growth medium is aspirated from confluent cell monolayers and replaced with 100 μL per well Tyrode's solution (in mM: 130 NaCl, 4 KCl, 1.8 CaCl₂, 1.0 MgCl₂, 20 HEPES, 10 glucose, pH 7.35) and 50 μL per well FLIPR Calcium Assay kit, component A (#R-8033, Molecular Devices Corporation) and incubated for 60 min. in a 5% CO₂ 37° C. incubator. 50 μL per well test compounds are added to the plates and further incubated for 15 min. in a 5% CO₂ 37° C. incubator. All final solutions contain the anion exchange inhibitor, probenecid (2.5 mM). The 96-well plates are then placed in the center position of the FLIPR 1 (Fluorometric Imaging Plate Reader, Molecular Devices Corporation). Cell monolayers in each well are simultaneously illuminated at 488 nm with an Argon ion laser, and fluorescence emission is monitored using a 510-570 nm bandpass filter and a cooled CCD camera. To depolarize the plasma membrane and activate L-type calcium channels, 50 μL per well of 20 mM KCl (final concentration) are dispensed simultaneously to all 96 wells using the FLIPR's automatic 96-well pipettor. Fluorescence measurements are captured for 5 min. following KCl addition. Calcium influx, expressed as % control, is calculated for each concentration of test compound and concentration-response curves and IC₅₀ values are generated using GraphPad Prism 4.0.

### Kv1.5 Patch Clamp EP

Kv1.5 currents are recorded by the whole cell mode of patch clamp electrophysiology.¹ Kv1.5 is stably over expressed in either HEK or LTK- cells. Microelectrodes are pulled from borosilicate glass (TW150) and heat polished (tip resistance, 1.5 to 3 megaohms). The external solution is standard Tyrodes solution. The internal (microelectrode) solution contained: 110 mM KCl, 5 mM K₂ATP, 5 mM K₄BAPTA, 1 mM MgCl₂ and 10 mM HEPES, adjusted to pH 7.2 with KOH. Command potentials are applied for 1 second to +60 mV from a holding potential of −70 mV using Axon software (pClamp 8.1) and hardware (Axopatch 1D, 200B). Compounds are prepared as 10-20 mM DMSO stocks and diluted to appropriate test concentrations. After stable currents are achieved, compounds are perfused onto the cells and the cells are pulsed every 5 seconds until no further changes in current are evident at a given compound concentration. Inhibition was measured at the end of the 1 second pulses and expressed relative to controls. Initial Kv1.5 inhibition is estimated by single point determinations done at 1 μM. Concentration response curves are generated for appropriate compounds utilizing at least four concentrations and an n=3. Curve fitting and IC₅₀ estimating are done using Graphpad software (Ver. 4).

### HERG Patch Clamp EP

HERG currents are recorded by the whole cell mode of patch clamp electrophysiology as described by Hamill et al.³ HERG is stably over expressed in HEK cells. Microelectrodes are pulled from borosilicate glass (TW150) and heat polished (tip resistance, 1.5 to 3 megaohms). The external solution is standard Tyrodes solution. The internal (microelectrode) solution contained: 110 mM KCl, 5 mM K₂ATP, 5 mM K₄BAPTA, 1 mM MgCl₂ and 10 mM HEPES, adjusted to pH 7.2 with KOH. Command potentials are applied for 2 seconds to +20 mV from a holding potential of −80 mV using Axon software (pClamp 8.1) and hardware (Axopatch 1D, 200B). Tail currents are generated by returning to −40 mV for 2 seconds. Compounds are prepared as 10-20 mM DMSO stocks and diluted to appropriate test concentrations. After stable currents are achieved, compounds are perfused onto the cells and the cells are pulsed every 20 seconds until no further changes in current are evident at a given compound concentration. Inhibition of HERG is measured at the peak of the tail currents and expressed relative to controls. Initial HERG activity is estimated by single point determinations run at 10 μM. Concentration response curves are generated for appropriate compounds utilizing at least four concentrations and an n=3. Curve fitting and IC₅₀ estimating are done using Graphpad software (Ver. 4). (Claycomb et al., Proc Natl Acad Sci USA 1998 Mar. 17; 95(6):2979-84; Xia M et al., J. Mol. Cell Cardiol., 204 January; 3(1): 111-9; Hamill et al., Pflugers Archiv. 391:85, 1981).

Results for representative compounds according to the present invention are listed in Table XIV below.

TABLE XIV

| | ¹Kv1.5% | ²L- | ³HERG | | |
|---|---|---|---|---|---|
| Compound | inhibition @ | type Ca²⁺ | % Inhib. @ | ¹Kv1.5 | ³HERG |
| Number | 1.0 μM | IC₅₀ μM | 10 μM | IC₅₀ nM | IC₅₀ μM |
| 1 | 2 | | | | |
| 2 | 6 | | | | |
| 3 | 6 | | | | |
| 4 | 94 | 8 | 64 | 151 | 9.5 |
| 5 | 84 | 36 | 8 | 514 | 31 |
| 6 | 68 | 13 | 85 | | |
| 7 | 87 | 9 | 12 | 579 | 23.5 |
| 8 | 91 | 11 | 25 | 350 | |
| 9 | 96 | 6.2 | 60 | | |
| 10 | 87 | 12 | 5 | | |
| 11 | 29 | | 33 | | |
| 12 | 70 | 8 | 40 | 611 | |
| 13 | 46 | | 57 | | |
| 14 | 59 | 15 | 75 | | |
| 15 | 84 | 2.5 | | | |
| 16 | 92 | 13 | 48 | 319 | |
| 17 | 31 | | | | |
| 18 | 10 | | | | |
| 19 | 97 | 5.3 | | | |
| 20 | 93 | 8 | 86 | 186 | 4 |
| 21 | 97 | 11 | 54 | | |
| 22 | 51 | 18 | 58 | | |
| 23 | 96 | 6.6 | 77 | 181 | 6.2 |
| 24 | 82 | 13 | 27 | | |
| 25 | 82 | 17 | 23 | | |
| 26 | 65 | 24 | 5 | | |
| 27 | 87 | 20 | 35 | | |
| 28 | 88 | 8.4 | | | |
| 29 | 83 | 8 | 31 | | |
| 30 | 93 | 21 | 91 | 166 | 6 |
| 31 | 75 | 26 | 25 | | |
| 32 | 90 | 13 | 57 | | |
| 33 | 80 | 25 | 54 | | |
| 34 | 80 | 18 | 50 | | |
| 35 | 96 | 30 | 65 | | |
| 36 | 92 | 7.4 | | | |
| 37 | 83 | 14 | 12 | | |
| 38 | 28 | | | | |
| 39 | 86 | 4.4 | | | |
| 40 | 22 | | | | |
| 41 | 17 | | | | |
| 42 | 94 | 23 | 81 | | |
| 43 | 23 | | | | |
| 44 | 73 | 15 | 61 | | |
| 45 | 77 | 6.2 | 87 | 287 | 3.3 |
| 46 | 68 | 17 | 32 | | |
| 47 | 75 | 20 | | | |
| 49 | 86 | 14 | 67 | 321 | 7.7 |
| 50 | 72 | 16 | 37 | | |
| 51 | 73 | 10 | | | |
| 52 | 89 | 12 | 79 | | |
| 53 | 93 | 4.5 | 77 | | |
| 54 | 88 | 18 | 53 | 266 | 4.9 |
| 55 | 64 | 8 | 71 | | |
| 56 | 12 | | | | |
| 57 | 25 | | | | |
| 58 | 92 | 12 | 68 | 250 | 4.4 |
| 59 | 11 | | | | |
| 60 | 32 | | | | |
| 62 | 93 | 7.8 | 62 | 257 | 4.6 |
| 63 | 73 | 3 | 40 | | |
| 64 | 36 | | | | |
| 65 | 80 | 3.6 | | | |
| 67 | 91 | 10 | 80 | | |
| 68 | 28 | | | | |
| 69 | 5 | | | | |
| 70 | 42 | | | | |
| 72 | 16 | | | | |
| 73 | 11 | | | | |
| 73 | 11 | | | | |
| 74 | 5 | | | | |
| 75 | 94 | 19 | 2 | | |
| 76 | 5 | | | | |
| 77 | 7 | | | | |
| 78 | 16 | | | | |
| 79 | 8 | | | | |
| 80 | 74 | 19 | 24 | | |
| 81 | 80 | 9 | 95 | | |
| 82 | 88 | 7 | 90 | | |
| 83 | 93 | 6.8 | 84 | | |
| 84 | 7 | | | | |
| 85 | 85 | 6.7 | 3 | | |
| 86 | 4 | | | | |
| 87 | 5 | | | | |
| 88 | 13 | | | | |
| 89 | 94 | 12 | 43 | 273 | 12 |
| 90 | 8 | | | | |
| 91 | 87 | 13 | 72 | | |
| 92 | 81 | 14 | 73 | | |
| 93 | 21 | | | | |
| 94 | 12 | | | | |
| 96 | 96 | 12 | 12 | 393 | |
| 97 | 16 | | | | |
| 98 | 90 | 13 | 13 | 320 | 13 |
| 99 | 80 | 24 | 24 | 397 | 16 |

¹Kv1.5 Patch Clamp EP as described herein
²FLIPR L-type Calcium Channel Assay as described herein
³HERG Patch Clamp EP as described herein

The following are additional methods that can be used to determine the suitability of the compounds of the present invention for use as Kv1.5 potassium channel inhibitors.

### In Vivo Test

Vehicle: Compounds are dissolved to a final concentration of 20-50 mg/ml, first in dimethyl acetamide (DMAC) then adding the balance of propylene glycol 200 (PEG200) for a ratio of DMAC/PEG200 (1:4).

Guinea Pig:(400-600 g) The animals are induced and maintained at a surgical plane of anesthesia with isoflurane at 1.5-2%. An incision is made in the neck and the carotid and jugular are isolated. Transducer-tipped catheters are introduced into the aorta and the left ventricle. A line for compound infusion is placed in the jugular. After 30 minutes for stabilization of the preparation the first dose is infused over 15 minutes followed by 10 minutes recovery before the pattern is repeated for the second and third doses. The animal is monitored continuously for heart rate, blood pressure, ECG, left ventricular pressure, the first derivative of LV pressure maximum and minimum, body temperature and exhaled Pco2.

Miniswine: The animals are induced with an IM injection of ketamine/xylazine followed briefly by 1-1.5% isoflurane if needed for introduction of a line into the vena cava in the neck. Following intubation, anesthesia is maintained with IV pentobarbital alone with boluses given every 30 minutes during the study. Two electrode-tipped catheters are introduced via the jugular, one into the right atrium and the other into the right ventricle. The carotid artery is isolated and a transducer-tipped catheter introduced into the left ventricle. An incision in the groin is used to access the femoral artery and vein. The artery is cannulated to monitor arterial pressure at the lower aorta and the vein is cannulated with an electrode-tipped catheter advanced into the right atrium. An incision is made above the fourth intercostal space and the ribs separated for access to the heart. The pericardium is opened and the left atrium is loosely clamped to the chest wall. A sensing and two pacing electrodes are placed on the atrium. The arterial pressure, ECG, LV pressure, atrial electrogram, body temperature, and exhaled Pco2 are monitored continuously.

When the surgical preparation is stable, baseline effective refractory periods (ERPs) are determined at paced rates of 150, 200, 240, and 300 beats per minute from the right and left atriums, and the right ventricle. Compound is then infused over 15 minutes and the ERP determinations are repeated starting at the 12th minute of the infusion. The animal is allowed to stabilize, then about 15 minutes after the first dose a second dose is given over 15 minutes followed by ERPs. A third dose may be given. After the final dose the ERPs are determined every 15 minutes until the values are back at baseline. Blood samples are collected at baseline, at the end of each dose, and 15 minutes after the final dose.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound having formula (I): 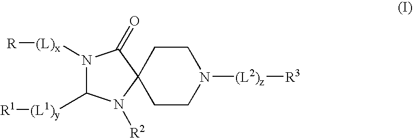
wherein R is optionally substituted phenyl;
R¹ is optionally substituted phenyl;
R² is optionally substituted C₁-C₆ linear or branched alkyl, optionally substituted C₃-C₆ cycloalkyl or —C(O)R²³, wherein R²³ is optionally substituted C₁-C₆ linear or branched alkyl or optionally substituted C₃-C₆ cycloalkyl;
R³ is selected from:
i) hydrogen;
ii) optionally substituted C₁-C₆ linear or branched alkyl, or optionally substituted C₃-C₆ cycloalkyl;
iii) —C(O)R⁴;
wherein R⁴ is optionally substituted C₁-C₆ linear or branched alkyl, optionally substituted C₃-C₆ cycloalkyl, optionally substituted heterocycle, optionally substituted aryl, or optionally substituted heteroaryl;
iv) —C(O)NR⁵R⁶;
wherein R⁵ and R⁶ are each independently selected from:
a) hydrogen;
b) optionally substituted C₁-C₆ linear or branched alkyl;
c) optionally substituted C₃-C₇ cycloalkyl;
d) —OR⁷;
wherein R⁷ is hydrogen or optionally substituted C₁-C₆ linear or branched alkyl;
e) —NR⁸R⁹;
wherein R⁸ and R⁹ are each independently hydrogen, optionally substituted C₁-C₆ linear or branched alkyl, optionally substituted C₁-C₆ linear or branched alkoxy, —OH, or —CO₂R¹⁰ wherein R¹⁰ is optionally substituted C₁-C₆ linear or branched alkyl; or R⁸ and R⁹ can be taken together with the atom to which they are bound to form an optionally substituted ring having from 3 to 7 ring atoms and optionally containing one or more additional heteroatom ring atoms independently selected from N, O, or S; or
f) R⁵ and R⁶ can be taken together with the atom to which they are bound to form an optionally substituted ring having from 3 to 7 ring atoms and optionally containing one or more additional heteroatom ring atoms independently selected from N, O, or S;
v) —C(NR¹¹)R¹²;
wherein R¹¹ is
a) hydrogen;
b) optionally substituted C₁-C₆ linear or branched alkyl, or optionally substituted C₃-C₆ cycloalkyl;
c) —OH; or
d) —CN; and
R¹² is
a) optionally substituted C₁-C₆ linear or branched alkyl, or optionally substituted C₃-C₆ cycloalkyl;
b) —OR¹³;
wherein R¹³ is hydrogen, optionally substituted C₁-C₆ linear or branched alkyl, optionally substituted C₃-C₆ cycloalkyl, or optionally substituted aryl; or
c)—NR¹⁴R¹⁵;
wherein R¹⁴ and R¹⁵ are each independently hydrogen, optionally substituted aryl, optionally substituted C₁-C₆ linear or branched alkyl, or optionally substituted C₃-C₈ cycloalkyl;
vi) —SO₂R¹⁶;
wherein R¹⁶ is optionally substituted aryl, optionally substituted C₁-C₆ linear or branched alkyl, or optionally substituted C₃-C₆ cycloalkyl;
vii) —C(O)R¹⁷;
wherein R¹⁷ is optionally substituted aryl or optionally substituted heteroaryl; or
viii) —C(O)OR¹⁸;
wherein R¹⁸ is optionally substituted C₁-C₆ linear or branched alkyl, optionally substituted C₃-C₆ cycloalkyl, or optionally substituted aryl;
L, L¹, and L² are linking units each independently a unit having the formula:
—[C(R¹⁹)₂]ₙ—
wherein each R¹⁹ is, at each occurrence, independently chosen from hydrogen, methyl, or ethyl;
n is 1 to 4; and
x, y, and z are each independently 0 or 1;
or a pharmaceutically acceptable salt form thereof.

2. The compound according to claim 1 wherein R is para-substituted phenyl.

3. The compound according to claim 1 wherein R¹ is para-substituted phenyl.

4. The compound according to claim 1 wherein R is phenyl optionally substituted with from 1 to 5 substituents independently selected from halogen, optionally substituted C₁-C₆ linear or branched alkyl, optionally substituted C₁-C₆ linear or branched haloalkyl, optionally substituted C₃-C₈ cycloalkyl, —OR²⁰, —CN, —N(R²⁰)₂, —CO₂R²⁰, —C(O)N(R²⁰)₂, —NR²C(O)R²⁰, —NO₂, or —SO₂R²; each R²⁰ is independently hydrogen, optionally substituted C₁-C₆ linear or branched alkyl, optionally substituted C₁-C₆ linear or branched haloalkyl, optionally substituted C₃-C₈ cycloalkyl, optionally substituted aryl, optionally substituted heterocycle, or optionally substituted heteroaryl; or two R²⁰ units can be taken together with the atom to which they are bound to form an optionally substituted ring having from 3 to 7 ring atoms and optionally containing one or more additional heteroatom ring atoms independently selected from N, O, or S.

5. The compound according to claim 4 wherein the optionally substituted alkyl, cycloalkyl, aryl, heterocycle, or heteroaryl groups are optionally substituted with from 1 to 5 substituents independently selected from —OR²²; —C(O)R²²; —C(O)OR²²—C(O)N(R²²)₂; —N(R²²)₂; —NR²²COR²²; halogen; C₁-C₆ linear or branched haloalkyl; —SO₂R²²; —SO₂N(R²²)₂; C₁-C₆ linear or branched alkyl; C₃-C₆ cycloalkyl; cyano; or nitro wherein two R²² units can be taken together to form a ring comprising 3-7 ring atoms or each R²² is independently hydrogen, C₁-C₆ linear or branched, alkyl, or C₃-C₆ cycloalkyl.

6. The compound according to claim 4 wherein R is phenyl, 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 3,4,5-trifluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 2,3,4-trichlorophenyl, 2,3,5-trichlorophenyl, 2,3,6-trichlorophenyl, 2,4,5-trichlorophenyl, 2,4,6-trichlorophenyl, or 3,4,5-trichlorophenyl.

7. The compound according to claim 4 wherein R is 2-methylphenyl, 3-methylphenyl, 4-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 3,4,5-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 3,5-diethylphenyl, 2,3,4-triethylphenyl, 2,3,5-triethylphenyl, 2,3,6-triethylphenyl, 2,4,5-triethylphenyl, 2,4,6-triethylphenyl, or 3,4,5-triethylphenyl.

8. The compound according to claim 4 wherein R is 2-cyclopropylphenyl, 3-cyclopropylphenyl, 4-cyclopropylphenyl, 2-(cyclopropylmethyl)phenyl, 3-(cyclopropylmethyl)phenyl, 4-(cyclopropylmethyl)phenyl, 2-iso-butylphenyl, 3-iso-butylphenyl, 4-iso-butylphenyl, 2-tert-butylphenyl, 3-tert-butylphenyl, 4-tert-butylphenyl, 2-cyclobutylphenyl, 3-cyclobutylphenyl, 4-cyclobutylphenyl, 2-(cyclobutylmethyl)phenyl, 3-(cyclobutylmethyl)phenyl, or 4-(cyclobutyl-methyl)phenyl.

9. The compound according to claim 4 wherein R is 2-methoxyphenyl, 3-methoxyphenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 3,5-dimethoxyphenyl, 2,3,4-trimethoxyphenyl, 2,3,5-trimethoxyphenyl, 2,3,6-trimethoxy-phenyl, 2,4,5-trimethoxyphenyl, 2,4,6-trimethoxyphenyl, 2-hydroxyphenyl, 3-hydroxyphenyl, 4-hydroxyphenyl, 2,3-dihydroxyphenyl, 2,4-dihydroxyphenyl, 2,5-dihydroxyphenyl, 2,6-dihydroxyphenyl, 3,4-dihydroxyphenyl, 3,5-dihydroxyphenyl, 2,3,4-trihydroxyphenyl, 2,3,5-trihydroxy-phenyl, 2,3,6-trihydroxyphenyl, 2,4,5-trihydroxyphenyl, or 2,4,6-trihydroxy-phenyl.

10. The compound according to claim 4 wherein R is 2-fluoromethoxyphenyl, 2-difluoromethoxyphenyl, 2-trifluoromethoxyphenyl, 3-fluoromethoxyphenyl, 3-difluoromethoxyphenyl, 3-trifluoromethoxyphenyl, 4-fluoromethoxyphenyl, 4-difluoromethoxyphenyl, 4-trifluoromethoxyphenyl, 2,4-bis(fluoromethoxy)phenyl, 2,4-bis(difluoromethoxy)phenyl, 2,4-bis(trifluoromethoxy)phenyl, 3,5-bis(fluoromethoxy)phenyl, 3,5-bis(difluoromethoxy)phenyl, or 3,5-bis(trifluoromethoxy)phenyl.

11. The compound according to claim 4 wherein R is 2-cyanophenyl, 3-cyanophenyl, 4-cyanophenyl, 2,3-dicyanophenyl, 2,4-dicyanophenyl, 2,5-dicyanophenyl, 2,6-dicyanophenyl, 3,4-dicyanophenyl, 3,5-dicyanophenyl, 2,3,4-tricyanophenyl, 2,3,5-tricyanophenyl, 2,3,6-tricyanophenyl, 2,4,5-tricyanophenyl, 3,4,5-tricyanophenyl, 2,4,6-tricyanophenyl, 2,6-dimethyl-4-fluorophenyl, 2,6-dimethyl-3-fluorophenyl, 2,6-dimethyl-4-chlorophenyl, 2,6-di-tert-butyl-4-hydroxyphenyl, 2,6-difluoro-4-chlorophenyl, 2,6-difluoro-3-chlorophenyl, 2-hydroxy-4-methylphenyl, 2-hydroxy-5-methylphenyl, 2,6-dihydroxy-4-tert-butylphenyl, or 2,6-difluoro-4-cyanophenyl.

12. The compound according to claim 4 wherein R is 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, or 4-(N,N-diethylamino)phenyl.

13. The compound according to claim 1 wherein R¹ is phenyl optionally substituted with from 1 to 5 substituents independently selected from halogen, optionally substituted C₁-C₆ linear or branched alkyl, optionally substituted C₃-C₈ cycloalkyl, —OR²¹, —CN, —N(R²¹)₂, —CO₂R²¹, —C(O)N(R²¹)₂, —NR²C(O)R²¹, —NO₂, and —SO₂R²¹; each R²¹ is independently hydrogen, optionally substituted C₁-C₆ linear or branched alkyl, optionally substituted C₃-C₈ cycloalkyl, optionally substituted aryl, optionally substituted heterocycle, or optionally substituted heteroaryl; or two R²¹ units can be taken together to form a ring comprising from 3-7 ring atoms.

14. The compound according to claim 13 wherein the optionally substituted alkyl, cycloalkyl, aryl, heterocyle and heteroaryl groups are optionally substituted with from 1 to 5 substituents independently selected from —OR²²; —C(O)R²²; —C(O)OR²²—C(O)N(R²²)₂; —N(R²²)₂; —NR²²COR²²; halogen; C₁-C₄ linear or branched haloalkyl; —SO₂R²²; —SO₂N(R²²)₂; C₁-C₆ linear or branched alkyl; C₃-C₈ cycloalkyl; cyano; or nitro wherein two R²² units can be taken together to form a ring comprising 3-7 ring atoms or each R²² is independently hydrogen, C₁-C₆ linear or branched alkyl, or C₃-C₈ cycloalkyl.

15. The compound according to claim 13 wherein R¹ is phenyl, 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 3,4,5-trifluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 2,3,4-trichlorophenyl, 2,3,5-trichlorophenyl, 2,3,6-trichlorophenyl, 2,4,5-trichlorophenyl, 2,4,6-trichlorophenyl, or 3,4,5-trichlorophenyl.

16. The compound according to claim 13 wherein R¹ is 2-methylphenyl, 3-methylphenyl, 4-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 3,4,5-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 3,5-diethylphenyl, 2,3,4-triethylphenyl, 2,3,5-triethylphenyl, 2,3,6-triethylphenyl, 2,4,5-triethylphenyl, 2,4,6-triethylphenyl, 3,4,5-triethylphenyl 2-isopropylphenyl, 3-isopropylphenyl, or 4-isopropylphenyl.

17. The compound according to claim 13 wherein R¹ is 2-cyclopropylphenyl, 3-cyclopropylphenyl, 4-cyclopropyl-phenyl, 2-(cyclopropylmethyl)phenyl, 3-(cyclopropylmethyl)phenyl, 4-(cyclopropyl-methyl)phenyl, 2-iso-butylphenyl, 3-iso-butylphenyl, 4-iso-butylphenyl, 2-tert-butylphenyl, 3-tert-butylphenyl, 4-tert-butylphenyl, 2-cyclobutylphenyl, 3-cyclobutylphenyl, 4-cyclobutylphenyl, 2-(cyclobutylmethyl)phenyl, 3-(cyclobutylmethyl)phenyl, or 4-(cyclobutylmethyl)phenyl.

18. The compound according to claim 13 wherein R¹ is 2-methoxyphenyl, 3-methoxyphenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 2,3,4-trimethoxyphenyl, 2,3,5-trimethoxyphenyl, 2,3,6-trimethoxyphenyl, 2,4,5-trimethoxyphenyl, 2,4,6-trimethoxyphenyl, 2-fluoromethoxyphenyl, 2-difluoromethoxyphenyl, 2-trifluoromethoxyphenyl, 3-fluoromethoxyphenyl, 3-difluoromethoxyphenyl, 3-trifluoromethoxyphenyl, 4-fluoromethoxyphenyl, 4-difluoromethoxyphenyl, 4-trifluoromethoxyphenyl, 2,4-bis(fluoromethoxy)phenyl, 2,4-bis(difluoromethoxy)phenyl, 2,4-bis(trifluoromethoxy)phenyl, 3,5-bis(fluoromethoxy)phenyl, 3,5-bis(difluoromethoxy)phenyl, or 3,5-bis(trifluoromethoxy)phenyl.

19. The compound according to claim 1 wherein R² is —C(O)R²³, wherein R²³ is C₁-C₆ linear or branched alkyl or C₃-C₆ cycloalkyl.

20. The compound according to claim 1 wherein R² is C₁-C₆ linear or branched alkyl or C₃-C₆ cycloalkyl.

21. The compound according to claim 20 wherein R² is methyl.

22. The compound according to claim 20 wherein R² is ethyl, n-propyl, isopropyl, or cyclopropyl.

23. The compound according to claim 1 wherein R³ is —C(O)NR⁵R⁶ and R⁵ and R⁶ are each independently selected from
a) hydrogen;
b) C₁-C₆ linear or branched alkyl;
c) C₃-C₇ cycloalkyl;
d) —OR⁷, wherein R⁷ is hydrogen or C₁-C₆ linear or branched alkyl; or
e) —NR⁸R⁹, wherein R⁸ and R⁹ are each independently hydrogen, C₁-C₆ linear or branched alkyl, C₁-C₆ linear or branched alkoxy, —OH, or —CO₂R¹⁰, wherein R¹⁰ is C₁-C₆ linear or branched alkyl; or R⁵ and R⁶ can be taken together with the atom to which they are bound to form an optionally substituted ring having from 3 to 7 ring atoms and optionally containing one or more additional heteroatom ring atoms independently selected from N, O, or S.

24. The compound according to claim 23 wherein R⁵ and R⁶ are each independently selected from hydrogen, methyl, ethyl, n-propyl, iso-propyl, cyclopropyl, n-butyl, sec-butyl, iso-butyl, tert-butyl, cyclobutyl, cyclopentyl, cyclohexyl, or cycloheptyl.

25. The compound according to claim 23 wherein R³ is —C(O)NHCH₂CH₃, —C(O)NH₂, —C(O)NHCH₃, —C(O)N(CH₂CH₃)₂, —C(O)N(CH₃)₂, or —C(O)NH[CH(CH₃)₂].

26. The compound according to claim 23 wherein R³ is —C(O)NH₂, or —C(O)NHCH₃.

27. The compound according to claim 23 wherein R³ is —C(O)NR⁶OR⁷ or —C(O)NR⁶NR⁸R⁹ wherein R⁶ is hydrogen, methyl, ethyl, n-propyl, iso-propyl, n-butyl, sec-butyl, iso-butyl, or tert-butyl; R⁷ is hydrogen, methyl, ethyl, n-propyl, iso-propyl, n-butyl, sec-butyl, iso-butyl, or tert-butyl; and R⁸ and R⁹ are each independently selected from hydrogen, methyl, ethyl, n-propyl, iso-propyl, n-butyl, sec-butyl, iso-butyl, tert-butyl, methoxy, ethoxy, n-propoxy, iso-propoxy, n-butoxy, sec-butoxy, iso-butoxy, tert-butoxy or hydroxyl.

28. The compound according to claim 23 wherein R³ is —C(O)NR⁵R⁶, and R⁵ and R⁶ are taken together to form an optionally substituted ring having from 3 to 7 ring atoms.

29. The compound according to claim 28 wherein R⁵ and R⁶ are taken together to form a ring selected from aziridin-1-yl, azetidin-1-yl, pyrrolidin-1-yl, piperazin-1-yl, 4-methylpiperazin-1-yl, morpholin-4-yl, or piperidin-1-yl.

30. The compound according to claim 1 wherein R³ is —C(NR¹¹)R¹², wherein R¹¹ is hydrogen, methyl, ethyl, n-propyl, iso-propyl, cyclopropyl, n-butyl, sec-butyl, iso-butyl, tert-butyl, hydroxyl, or cyano; and R¹² is methyl, ethyl, n-propyl, iso-propyl, cyclopropyl, n-butyl, sec-butyl, iso-butyl, tert-butyl, —OR¹³, or —NR¹⁴R¹⁵, wherein R¹³ is hydrogen, methyl, ethyl, n-propyl, iso-propyl, cyclopropyl, n-butyl, sec-butyl, iso-butyl, tert-butyl, or phenyl; and R¹⁴ and R¹⁵ are each independently selected from hydrogen, methyl, ethyl, n-propyl, iso-propyl, cyclopropyl, n-butyl, sec-butyl, iso-butyl, or tert-butyl.

31. The compound according to claim 1 wherein R³ is —SO₂R¹⁶ and R¹⁶ is phenyl, methyl, ethyl, n-propyl, iso-propyl, cyclopropyl, n-butyl, sec-butyl, iso-butyl, or tert-butyl.

32. The compound according to claim 1 wherein R³ is —C(O)R¹⁷, wherein R¹⁷ is C₁-C₅ heteroaryl.

33. The compound according to claim 32 wherein R¹⁷ is selected from triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, or pyridin-4-yl.

34. The compound according to claim 1 wherein R³ is —C(O)OR¹⁸, wherein R¹⁸ is methyl, ethyl, n-propyl, iso-propyl, cyclopropyl, n-butyl, sec-butyl, iso-butyl, or tert-butyl.

35. The compound according to claim 34 wherein R³ is —C(O)OCH₃, —C(O)OCH₂CH₃, —C(O)OCH(CH₃)₂, or —C(O)OC(CH₃)₃.

36. The compound according to claim 1 wherein R³ is —C(O)R⁴, wherein R⁴ is methyl, ethyl, n-propyl, iso-propyl, cyclopropyl, n-butyl, sec-butyl, iso-butyl, or tert-butyl.

37. The compound according to claim 1 wherein x is 1 and L is —CH₂CH₂—.

38. The compound according to claim 1 wherein x is 1 and L is —CH₂CH₂CH₂—.

39. The compound according to claim 1 wherein x is 1 and L is —CH₂—.

40. The compound according to claim 1 wherein x is 0.

41. The compound according to claim 1 wherein y is 1 and L¹ is —CH₂—.

42. The compound according to claim 1 wherein y is 0.

43. The compound according to claim 1 wherein z is 1 and L² is —CH₂—.

44. The compound according to claim 1 wherein z is 0.

45. The compound according to claim 1 wherein x is 1, y is 0 and z is 0.

46. The compound according to claim 45 wherein L is —CH₂CH₂—.

47. The compound according to claim 1 wherein:
R³ is hydrogen, —C(O)R⁴; —C(O)NR⁵R⁶, —C(O)NR⁵OR⁷; —C(O)NR⁵NR⁸R⁹, —C(NR¹¹)R¹², —SO₂R¹⁶, —C(O)OR¹⁸, or —C(O)R¹⁷;
R⁴ is —CH₃, —CH₂CH₃, —CH₂CH₂CH₃, —CH(CH₃)₂, cyclopropyl, cyclobutyl, cyclopentyl, or cyclohexyl;
R⁵ is hydrogen, —CH₃, —CH₂CH₃, or —CH(CH₃)₂;
R⁶ is hydrogen, —CH₃, or —CH₂CH₃; or
R⁵ and R⁶ are taken together to form aziridin-1-yl, pyrrolidin-1-yl, piperidin-1-yl, 4-(methyl)piperazin-1-yl, morpholin-4-yl;
R⁷ is hydrogen, —CH₃, or —CH₂CH₃, R⁸ is hydrogen;
R⁹ is hydrogen, —C(O)OCH₃, or —C(O)OC(CH₃)₃;
R¹¹ is OH, or —CN;
R¹²—NH₂, —CH₃, or —NR¹⁴R¹⁵;
R¹⁴ is hydrogen, CH₃, or phenyl;
R¹⁵ is hydrogen, CH₃, or phenyl;
R¹⁶ is —CH₃, —CH₂CH₃, —CH(CH₃)₂, or —C₆H₅;
R¹⁸ is —CH₃, —CH₂CH₃, —CH(CH₃)₂, —C₆H₅, or —C(CH₃)₃; and
R¹⁷ is imidazolin-1-yl, isoxazolin-5-yl, furan-2-yl, thiophen-2-yl, azetidin-1-yl, pyrrolidin-1-yl, piperidin-1-yl, piperazin-1-yl, or morpholin-4-yl.

48. The compound according to claim 47 wherein R² is C₁-C₆ linear or branched alkyl, or C₃-C₆ cycloalkyl.

49. The compound according to claim 48 wherein R² is methyl.

50. The compound according to claim 48 wherein R and R¹ are independently selected from phenyl optionally substituted with from 1 to 5 substituents independently selected from halogen, optionally substituted C₁-C₆ linear or branched alkyl, optionally substituted C₁-C₆ linear or branched haloalkyl, optionally substituted C₃-C₈ cycloalkyl, —OR²⁰, —CN, —N(R²⁰)₂, —CO₂R²⁰, —C(O)N(R²)₂, —NR²C(O)R²⁰, —NO₂, or —SO₂R²; each R²⁰ is independently hydrogen, optionally substituted C₁-C₆ linear or branched alkyl, optionally substituted C₁-C₆ linear or branched haloalkyl, or optionally substituted C₃-C₈ cycloalkyl; or two R²⁰ units can be taken together with the atom to which they are bound to form an optionally substituted ring having from 3 to 7 ring atoms and optionally containing one or more additional heteroatom ring atoms independently selected from N, O, or S.

51. The compound according to claim 48, wherein R and R¹ are each para-substituted.

52. The compound according to claim 50 wherein R and R¹ are independently selected from 4-methoxyphenyl, 4-trifluoromethylphenyl, 4-cyclopropylphenyl, 4-diethylaminophenyl, 4-difluoromethoxyphenyl, phenyl, or 4-tert-butylphenyl.

53. The compound according to claim 48 wherein R³ is hydrogen, —C(O)CH₃, —C(O)cyclopropyl, —C(O)NH₂, —C(O)NHCH₃, —C(O)N(CH₃)₂, —C(O)NH[CH(CH₃)₂], —C(O)NHCH₂CH₃, —C(O)N(CH₂CH₃), —C(O)OCH₃, —C(O)OCH₂CH₃, —C(O)OCH(CH₃)₂, —C(O)OC(CH₃)₃, —C(O)NHOH, —C(O)NHOCH₃, —C(O)N(CH₃)OCH₃, —C(O)NHNH₂, —C(O)NHOCH₂CH₃, —C(O)NCH₃OCH₃, —C(O)NHNHC(O)OCH₃, —C(O)NHNHC(O)OC(CH₃)₃, —C(NCN)NH₂, —C(NCN)NHCH₃, —C(NCN)NHC₆H₅, —C(O)aziridin-1-yl, —C(O)azetidin-1-yl, —C(O)pyrrolidin-1-yl, —C(O)piperidin-1-yl, —C(O)piperazin-1-yl, —C(O)morpholin-4-yl, —C(O)imidazolin-1-yl, —C(O)isoxazolin-5-yl, —SO₂CH₃, —SO₂CH₂CH₃, —SO₂CH(CH₃)₂, or SO₂C₆H₅.

54. The compound according to claim 1 having formula (X): 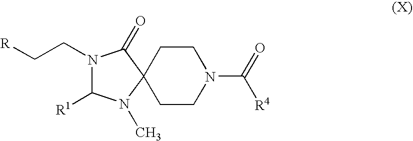
wherein R is optionally substituted phenyl, said substitution selected from
i) halogen;
ii) C₁-C₆ linear or branched alkyl or C₃-C₆ cycloalkyl;
iii) —OR²⁰;
iv) —CN;
v) —N(R²⁰)₂;
vi) —CO₂R²⁰;
vii) —C(O)N(R²⁰)₂;
viii) —NR²⁰C(O)R²⁰;
ix) —NO₂; or
x) —SO₂R²⁰;
wherein each R²⁰ is independently hydrogen, optionally substituted C₁-C₆ linear or branched alkyl, or optionally substituted C₃-C₆ cycloalkyl; or two R²⁰ units can be taken together to form a ring comprising from 3-7 ring atoms;
R¹ is optionally substituted phenyl, said substitution selected from
i) halogen;
ii) C₁-C₆ linear or branched alkyl or C₃-C₆ cycloalkyl;
iii) —OR²¹;
iv) —CN;
v) —N(R²¹);
vi) —CO₂R²¹;
vii) —C(O)N(R²¹)₂;
viii) —NR²¹C(O)R²¹;
ix) —NO₂; or
x) —SO₂R²¹;
wherein each R²¹ is independently hydrogen, optionally substituted C₁-C₆ linear or branched alkyl, or optionally substituted C₃-C₆ cycloalkyl; or two R²¹ units can be taken together to form a ring comprising from 3-7 ring atoms; and
R⁴ is optionally substituted C₁-C₆ linear or branched alkyl, or optionally substituted C₃-C₆ cycloalkyl; or a pharmaceutically acceptable salt form thereof.

55. The compound according to claim 1 having the formula (XI): 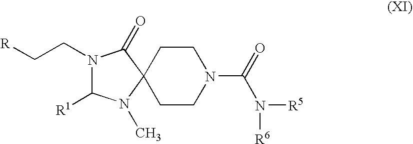
wherein R is optionally substituted phenyl, said substitution selected from
i) halogen;
ii) C₁-C₆ linear or branched alkyl or C₃-C₆ cycloalkyl;
iii) —OR²⁰;
iv) —CN;
v) —N(R²⁰)₂;
vi) —CO₂R²⁰;
vii) —C(O)N(R²⁰)₂;
viii) —NR²C(O)R²⁰;
ix) —NO₂; or
x) —SO₂R²⁰;
wherein each R²⁰ is independently hydrogen, optionally substituted C₁-C₆ linear or branched alkyl, or optionally substituted C₃-C₆ cycloalkyl; or two R²⁰ units can be taken together to form a ring comprising from 3-7 ring atoms;
R¹ is optionally substituted phenyl, said substitution selected from
i) halogen;
ii) C₁-C₆ linear or branched alkyl or C₃-C₆ cycloalkyl;
iii) —OR²¹;
iv) —CN;
v) —N(R²¹)₂;
vi) —CO₂R²¹;
vii) —C(O)N(R²¹)₂;
viii) —NR²¹C(O)R²¹;
ix) —NO₂; or
x) —SO₂R²¹;
wherein each R²¹ is independently hydrogen, optionally substituted C₁-C₆ linear or branched alkyl, or optionally substituted C₃-C₆ cycloalkyl; or two R²¹ units can be taken together to form a ring comprising from 3-7 ring atoms; and
R⁵ and R⁶ are each independently selected from
a) hydrogen;
b) C₁-C₆ linear or branched alkyl;
c) C₃-C₇ cyclic alkyl;
d) —OR⁷;
wherein R⁷ is hydrogen or C₁-C₆ linear or branched alkyl;
e) —NR⁸R⁹;
wherein R⁸ and R⁹ are each independently hydrogen, C₁-C₆ linear or branched alkyl, C₁-C₆ linear or branched alkoxy, —OH, or —CO₂R¹⁰ wherein R¹⁰ is C₁-C₆ linear or branched alkyl; or
f) R⁵ and R⁶ can be taken together to from a ring having from 3 to 7 ring atoms; or a pharmaceutically acceptable salt form thereof.

56. The compound according to claim 1 having the formula (XII) or (XIII): 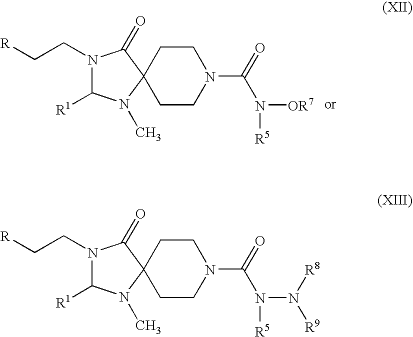
wherein R is optionally substituted phenyl, said substitution selected from
i) halogen;
ii) C₁-C₆ linear or branched alkyl or C₃-C₆ cycloalkyl;
iii) —OR²⁰;
iv) —CN;
v) —N(R²⁰);
vi) —CO₂R²⁰;
vii) —C(O)N(R²⁰)₂;
viii) —NR²⁰C(O)R²⁰;
ix) —NO₂; or
x) —SO₂R²⁰;
each R²⁰ is independently hydrogen, optionally substituted C₁-C₆ linear, branched, alkyl or optionally substituted C₃-C₆ cycloalkyl; or two R²⁰ units can be taken together to form a ring comprising from 3-7 ring atoms;
R¹ is optionally substituted phenyl, said substitution selected from
i) halogen;
ii) C₁-C₆ linear or branched alkyl or C₃-C₆ cycloalkyl;
iii) —OR²⁰;
iv) —CN;
v) —N(R²¹);
vi) —CO₂R²¹;
vii) —C(O)N(R²¹)₂;
viii) —NR²¹C(O)R²¹;
ix) —NO₂; or
x) —SO₂R²¹;
wherein each R²¹ is independently hydrogen, optionally substituted C₁-C₆ 1 linear or branched alkyl or optionally substituted C₃-C₆ cycloalkyl; or two R²¹ units can be taken together to form a ring comprising from 3-7 ring atoms; and
R⁵ and R⁶ are each independently selected from
a) hydrogen;
b) C₁-C₆ linear or branched alkyl;
c) C₃-C₇ cyclic alkyl;
d) —OR⁷;
wherein R⁷ is hydrogen or C₁-C₆ linear or branched alkyl;
e) —NR⁸R⁹;
wherein R⁸ and R⁹ are each independently hydrogen, C₁-C₆ linear or branched alkyl, C₁-C₆ linear or branched alkoxy, —OH, or —CO₂R¹⁰ wherein R¹⁰ is C₁-C₆ linear or branched alkyl; or
f) R⁵ and R⁶ can be taken together to from a ring having from 3 to 7 ring atoms, or a pharmaceutically acceptable salt form thereof.

57. The compound according to claim 1 having the formula (XXII): 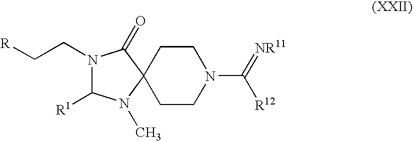
wherein R¹¹ is
a) hydrogen;
b) C₁-C₆ linear or branched alkyl;
c) —OH; or
d) —CN;
R¹² is
a) C₁-C₆ linear or branched alkyl;
b) —OR¹³;
wherein R¹³ is hydrogen, C₁-C₆ linear or branched alkyl, or phenyl; or
c) —NR¹⁴R¹⁵; and
R¹⁴ and R¹⁵ are each independently hydrogen, C₁-C₆ linear or branched alkyl, or a pharmaceutically acceptable salt form thereof.

58. The compound according to claim 1 having the formula (XVI): 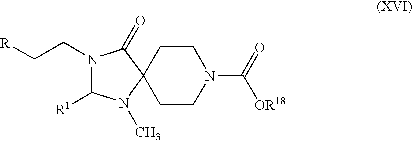
wherein R is optionally substituted phenyl, said substitution selected from
i) halogen;
ii) C₁-C₆ linear or branched alkyl or C₃-C₆ cycloalkyl;
iii) —OR²⁰;
iv) —CN;
v) —N(R²⁰);
vi) —CO₂R²⁰;
vii) —C(O)N(R²⁰)₂;
viii) —NR²C(O)R²⁰;
ix) —NO₂; or
x) —SO₂R²⁰;
each R²⁰ is independently hydrogen, optionally substituted C₁-C₆ linear or branched alkyl or optionally substituted C₃-C₆ cycloalkyl; or two R²⁰ units can be taken together to form a ring comprising from 3-7 ring atoms;
R¹ is optionally substituted phenyl, said substitution selected from
i) halogen;
ii) C₁-C₆ linear or branched alkyl or C₃-C₆ cycloalkyl;
iii) —OR²¹;
iv) —CN;
v) —N(R²¹)₂;
vi) —CO₂R²¹;
vii) —C(O)N(R²¹)₂;
viii) —NR²¹C(O)R²¹;
ix) —NO₂; or
x) —SO₂R²¹;
wherein each R²¹ is independently hydrogen, optionally substituted C₁-C₆ linear or branched alkyl or optionally substituted C₃-C₆ cycloalkyl; or two R²¹ units can be taken together to form a ring comprising from 3-7 ring atoms; and
R¹⁸ is C₁-C₆ linear or branched alkyl; or a pharmaceutically acceptable salt form thereof.

59. The compound according to claim 1 wherein the alkyl, alkoxy, cycloalkyl, aryl, heterocycle and heteroaryl groups are optionally substituted with from 1 to 5 substituents independently selected from —OR²²; —C(O)R²²; —C(O)OR²²; —C(O)N(R²²)₂; —N(R²²)₂; —NR²²COR²²; halogen; C₁-C₆ linear or branched haloalkyl; —SO₂R²²; —SO₂N(R²²)₂; C₁-C₆ linear or branched alkyl optionally substituted with C₃-C₆ cycloalkyl; C₃-C₆ cycloalkyl; cyano; or nitro wherein two R²² units can be taken together to form a ring comprising 3-7 ring atoms or each R²² is independently hydrogen, C₁-C₆ linear or branched alkyl, C₁-C₆ linear or branched haloalkyl, or C₃-C₆ cycloalkyl.

60. The compound according to claim 1 wherein the alkyl, alkoxy, cycloalkyl, aryl, heterocycle and heteroaryl groups are optionally substituted from 1 to 3 substituents independently selected from C₁-C₆ linear or branched alkyl, C₃-C₆ cycloalkyl, halogen, or C₁-C₆ haloalkyl.

61. A compound according to claim 1 wherein the optionally substituted heterocyle and optionally substituted heteroaryl groups are independently selected from optionally substituted pyrrolidinyl, optionally substituted aziridinyl, optionally substituted azetidinyl, optionally substituted pyrrolidinyl, optionally substituted piperazinyl, optionally substituted morpholinyl, optionally substituted piperidinyl, optionally substituted imidazolyl, optionally substituted isoxazolinyl, optionally substituted furanyl, optionally substituted thiophenyl, optionally substituted pyrimidinyl, optionally substituted pyridinyl, optionally substituted triazinyl, or optionally substituted thiazolyl.

62. A compound according to claim 1 wherein the optionally substituted heterocycle of R⁴ is pyrrolidinyl; the optionally substituted ring having from 3 to 7 ring atoms of R⁵ and R⁶ is aziridinyl, azetidinyl, pyrrolidinyl, piperazinyl, 4-methylpiperazinyl, morpholinyl or piperidinyl; and the optionally substituted heteroaryl of R¹⁷ is imidazolyl, isoxazolinyl, furanyl, thiophenyl, pyrimidinyl, pyridinyl, triazinyl, or thiazolyl.

63. A compound of claim 1 that is:
2-(4-Cyclopropylphenyl)-3-[2-(4-methoxyphenyl)ethyl]-1-methyl-1,3,8-triaza-spiro[4.5]decan-4-one;
2-(4-tert-Butylphenyl)-3-[2-(4-methoxyphenyl)ethyl]-1-methyl-1,3,8-triaza-spiro[4.5]decan-4-one;
2-(4-Difluoromethoxyphenyl)-3-[2-(4-methoxyphenyl)ethyl]-1-methyl-1,3,8-triaza-spiro[4.5]decan-4-one;
8-Cyclopropylcarbonyl-2-(4-cyclopropylphenyl)-3-[2-(4-methoxyphenyl)ethyl]-1-methyl-1,3,8-triaza-spiro[4.5]decan-4-one;
8-Cyclopropylcarbonyl-2-(4-difluoromethoxyphenyl)-3-[2-(4-methoxyphenyl) —ethyl]-1-methyl-1,3,8-triazaspiro[4.5]decan-4-one;
2-(4-Cyclopropylphenyl)-3-[2-(4-methoxyphenyl)ethyl]-1-methyl-4-oxo-1,3,8-triaza-spiro[4.5]decane-8-carboxylic acid amide;
2-(4-Cyclopropylphenyl)-3-[2-(4-methoxyphenyl)ethyl]-1-methyl-4-oxo-1,3,8-triaza-spiro[4.5]decane-8-carboxylic acid methyl amide;
2-(4-Cyclopropylphenyl)-3-[2-(4-methoxyphenyl)ethyl]-1-methyl-8-(piperidine-1-carbonyl)-1,3,8-triaza-spiro[4.5]decan-4-one;
2-[4-(Diethylamino)phenyl]-3-[2-(4-methoxyphenyl)ethyl]-1-methyl-4-oxo-1,3,8-triaza-spiro[4.5]decane-8-carboxylic acid amide;
2-(4-Trifluoromethylphenyl)-3-[2-(4-methoxyphenyl)ethyl]-1-methyl-4-oxo-1,3,8-triaza-spiro[4.5]decane-8-carboxylic acid amide;
2-(4-tert-Butylphenyl)-3-[2-(4-methoxyphenyl)ethyl]-1-methyl-4-oxo-1,3,8-triaza-spiro[4.5]decane-8-carboxylic acid amide;
2-(4-Difluoromethoxyphenyl)-3-[2-(4-methoxyphenyl)ethyl]-1-methyl-4-oxo-1,3,8-triaza-spiro[4.5]decane-8-carboxylic acid amide;
2-(4-Cyclopropylphenyl)-3-[2-(4-trifluoromethoxyphenyl)ethyl]-1-methyl-4-oxo-1,3,8-triaza-spiro[4.5]decane-8-carboxylic acid amide;
2-(4-tert-Butylphenyl)-3-[2-(4-methoxyphenyl)ethyl]-1-methyl-4-oxo-1,3,8-triaza-spiro[4.5]decane-8-carboxylic acid ethyl amide;
2-(4-Cyclopropylphenyl)-3-[2-(4-methoxyphenyl)ethyl]-1-methyl-4-oxo-1,3,8-triaza-spiro[4.5]decane-8-carboxylic acid isopropylamide;
2-(4-Methoxyphenyl)-3-[2-(4-methoxyphenyl)ethyl]-1-methyl-4-oxo-1,3,8-triaza-spiro[4.5]decane-8-carboxylic acid isopropylamide;
2-(4-Cyclopropylphenyl)-3-[2-(4-methoxyphenyl)ethyl]-1-methyl-4-oxo-1,3,8-triaza-spiro[4.5]decane-8-carboxylic acid dimethylamide;
2-(4-Cyclopropylphenyl)-3-[2-(4-methoxyphenyl)ethyl]-1-methyl-4-oxo-1,3,8-triaza-spiro[4.5]decane-8-carboxylic acid diethylamide;
2-(4-Cyclopropylphenyl)-3-[2-(4-methoxyphenyl)ethyl]-1-methyl-4-oxo-1,3,8-triaza-spiro[4.5]decane-8-carboxylic acid cyclopentylamide;
2-(4-Cyclopropylphenyl)-3-[2-(4-methoxyphenyl)ethyl]-1-methyl-8-(azetidin-1-ylcarbonyl)-1,3,8-triaza-spiro[4.5]decan-4-one;
2-(4-Cyclopropylphenyl)-3-[2-(4-methoxyphenyl)ethyl]-1-methyl-8-[(4-methylpiperazin-1-yl)carbonyl]-1,3,8-triaza-spiro[4.5]decan-4-one;
2-(4-Cyclopropylphenyl)-3-[2-(4-methoxyphenyl)ethyl]-1-methyl-8-(pyrrolidin-1-yl-carbonyl)-1,3,8-triaza-spiro[4.5]decan-4-one;
2-(4-Cyclopropylphenyl)-3-[2-(4-methoxyphenyl)ethyl]-1-methyl-8-(morpholin-4-yl-carbonyl)-1,3,8-triaza-spiro[4.5]decan-4-one;
2-(4-Cyclopropylphenyl)-N-methoxy-3-[2-(4-methoxyphenyl)ethyl]-1-methyl-4-oxo-1,3,8-triazaspiro[4.5]decane-8-carboxamide;
tert-Butyl 2-({2-(4-cyclopropylphenyl)-3-[2-(4-methoxyphenyl)ethyl]-1-methyl-4-oxo-1,3,8-triazaspiro[4.5]dec-8-yl}carbonyl)hydrazine carboxylate;
2-(4-Cyclopropylphenyl)-N-hydroxy-3-[2-(4-methoxyphenyl)ethyl]-1-methyl-4-oxo-1,3,8-triazaspiro[4.5]decane-8-carboxamide;
2-(4-Cyclopropylphenyl)-N-ethoxy-3-[2-(4-methoxyphenyl)ethyl]-N,1-dimethyl-4-oxo-1,3,8-triazaspiro[4.5]decane-8-carboxamide;
2-(4-Cyclopropylphenyl)-N-methoxy-3-[2-(4-methoxyphenyl)ethyl]-N,1-dimethyl-4-oxo-1,3,8-triazaspiro[4.5]decane-8-carboxamide;
Phenyl N-cyano-3-(4-methoxyphenethyl)-2-(4-methoxyphenyl)-1-methyl-4-oxo-1,3,8-triazaspiro[4.5]decane-8-carbimidate;
(E)-N-′-cyano-2-(4-cyclopropylphenyl)-3-(4-methoxyphenethyl)-1-methyl-4-oxo-1,3,8-triazaspiro[4.5]decane-8-carboximidamide;
2-(4-Cyclopropylphenyl)-8-methanesulfonyl-3-[2-(4-methoxy-phenyl)ethyl]-1-methyl-1,3,8-triaza-spiro[4.5]decan-4-one;
2-(4-tert-Butylphenyl)-8-methanesulfonyl-3-[2-(4-methoxyphenyl)ethyl]-1-methyl-1,3,8-triaza-spiro[4.5]decan-4-one;
2-(4-Trifluoromethylphenyl)-8-methanesulfonyl-3-[2-(4-methoxyphenyl)ethyl]-1-methyl-1,3,8-triaza-spiro[4.5]decan-4-one;
Ethyl 2-(4-cyclopropylphenyl)-3-[2-(4-methoxyphenyl)ethyl]-1-methyl-4-oxo-1,3,8-triazaspiro[4.5]decane-8-carboxylate;
Isopropyl 2-(4-cyclopropylphenyl)-3-[2-(4-methoxyphenyl)ethyl]-1-methyl-4-oxo-1,3,8-triazaspiro[4.5]decane-8-carboxylate;
tert-Butyl 2-(4-methoxyphenyl)-3-[2-(4-methoxyphenyl)ethyl]-1-methyl-4-oxo-1,3,8-triazaspiro[4.5]decane-8-carboxylate;
tert-Butyl 2-(4-tert-butylphenyl)-3-[2-(4-methoxyphenyl)ethyl]-1-methyl-4-oxo-1,3,8-triazaspiro[4.5]decane-8-carboxylate;
tert-Butyl 2-(4-diethylaminophenyl)-3-[2-(4-methoxyphenyl)ethyl]-1-methyl-4-oxo-1,3,8-triazaspiro[4.5]decane-8-carboxylate;
tert-Butyl 2-(4-difluoromethoxyphenyl)-3-[2-(4-methoxyphenyl)ethyl]-1-methyl-4-oxo-1,3,8-triazaspiro[4.5]decane-8-carboxylate;
Methyl 2-(4-cyclopropylphenyl)-3-[2-(4-methoxyphenyl)ethyl]-1-methyl-4-oxo-1,3,8-triazaspiro[4.5]decane-8-carboxylate;
2-(4-Cyclopropylphenyl)-8-(isoxazol-5-yl-carbonyl)-3-[2-(4-methoxyphenyl)ethyl]-1-methyl-1,3,8-triazaspiro[4.5]decan-4-one;
2-(4-Cyclopropylphenyl)-1-methyl-3-(3-phenylpropyl)-1,3,8-triazaspiro[4,5]-decan-4-one;
8-Acetyl-2-(4-cyclopropylphenyl)-3-(3-phenylpropyl)-1-methyl-1,3,8-triazaspiro-[4.5]decan-4-one;
8-Cyclopropanecarbonyl-2-(4-cyclopropylphenyl)-3-(3-phenylpropyl)-1-methyl-1,3,8-triazaspiro-[4.5]decan-4-one;
8-Cyclopropanecarbonyl-2-(4-methoxyphenyl)-3-(3-phenylpropyl)-1-methyl-1,3,8-triazaspiro-[4.5]decan-4-one;
2-(4-Cyclopropylphenyl)-1-methyl-4-oxo-3-(3-phenylpropyl)-1,3,8-triazaspiro-[4.5]decane-8-carboxylic acid tert-butyl ester;
2-(4-tert-Butylphenyl)-1-methyl-4-oxo-3-(3-phenylpropyl)-1,3,8-triazaspiro-[4.5]decane-8-carboxylic acid tert-butyl ester;
2-(4-Cyclopropylphenyl)-1-methyl-4-oxo-3-(3-phenylpropyl)-1,3,8-triazaspiro-[4.5]decane-8-carboxylic acid amide;
2-(4-tert-Butylphenyl)-1-methyl-4-oxo-3-(3-phenylpropyl)-1,3,8-triazaspiro-[4.5]decane-8-carboxylic acid amide;
2-(4-Cyclopropylphenyl)-1-methyl-4-oxo-3-(3-phenylpropyl)-1,3,8-triazaspiro-[4.5]decane-8-carboxylic acid methylamide;
2-(4-Cyclopropylphenyl)-1-methyl-4-oxo-3-(3-phenylpropyl)-1,3,8-triazaspiro-[4.5]decane-8-carboxylic acid isopropyl amide;
2-(4-Cyclopropylphenyl)-1-methyl-4-oxo-3-(3-phenylpropyl)-1,3,8-triazaspiro-[4.5]decane-8-carboxylic acid dimethylamide;
2-(4-Cyclopropylphenyl)-8-methanesulfonyl-1-methyl-3-(3-phenylpropyl)-1,3,8-triazaspiro-[4.5]decan-4-one;
2-{2-(4-cyclopropylphenyl)-3-[2-(4-methoxyphenyl)ethyl]-1-methyl-4-oxo-1,3,8-triaza-spiro[4.5]dec-8-yl}acetamide;
8-cyclopropylmethyl-2-(4-cyclopropylphenyl)-3-[2-(4-methoxy-phenyl)ethyl]-1-methyl-1,3,8-triaza-spiro[4.5]decan-4-one;
2-{2-(4-Difluoromethoxyphenyl)-3-[2-(4-methoxyphenyl)ethyl]-1-methyl-4-oxo-1,3,8-triaza-spiro[4.5]dec-8-yl}acetamide;
2-(4-tert-Butylbenzyl)-3-[2-(4-methyoxyphenyl)ethyl]-1-methyl-4-oxo-1,3,8-triaza-spiro[4.5]decane-8-carboxylic acid tert-butyl ester;
2-(4-tert-Butylbenzyl)-3-[2-(4-methoxyphenyl)ethyl]-1-methyl-1,3,8-triazaspiro[4.5]decan-4-one;
2-(4-tert-Butylbenzyl)-3-[2-(4-methoxyphenyl)ethyl]-1-methyl-4-oxo-1,3,8-triazaspiro[4.5]decane-8-carboxylic acid amide;
(2-{2-(4-cyclopropylphenyl)-3-[2-(4-methoxyphenyl)ethyl]-1-methyl-4-oxo-1,3,8-triaza-spiro[4.5]dec-8-yl}-1,1-dimethyl-2-oxo-ethyl)-carbamic acid tert-butyl ester;
8-(2-Amino-2-methylpropionyl)-2-(4-cyclopropylphenyl)-3-[2-(4-methoxy-phenyl)ethyl]-1-methyl-1,3,8-triaza-spiro[4.5]decan-4-one;
8-Acetyl-2-(4-cyclopropylphenyl)-3-[2-(4-methoxyphenyl)ethyl]-1-methyl-1,3,8-triaza-spiro[4.5]decan-4-one;
N′-cyano-2-(4-methoxyphenyl)-3-[2-(4-methoxyphenyl)ethyl]-1-methyl-4-oxo-1,3,8-triazaspiro[4.5]decane-8-carboximidamide;
1-methyl-2-(4-trifluoromethylphenyl)-3-[2-(4-methoxyphenyl)-ethyl]-4-oxo-1,3,8-triaza-spiro[4.5]decane-8-carboxylic acid tert-butyl ester;
or a pharmaceutically acceptable salt form thereof.

64. A compound of claim 1 that is:
(E)-N-′-cyano-2-(4-cyclopropylphenyl)-3-(4-methoxyphenethyl)-1-methyl-4-oxo-1,3,8-triazaspiro[4.5]decane-8-carboximidamide;
2-(4-methoxyphenyl)-3-[2-(4-methoxyphenyl)ethyl]-1-methyl-4-oxo-1,3,8-triazaspiro[4.5]decane-8-carboxamide;
tert-butyl 2-(4-cyclopropylphenyl)-1-methyl-4-oxo-3-{2-[4-(trifluoromethoxy)phenyl]ethyl}-1,3,8-triazaspiro[4.5]decane-8-carboxylate;
2-(4-cyclopropylphenyl)-1-methyl-3-{2-[4-(trifluoromethoxy)phenyl]ethyl}-1,3,8-triazaspiro[4.5]decan-4-one;
tert-butyl 2-(4-cyclopropylphenyl)-3-[2-(4-methoxyphenyl)ethyl]-1-methyl-4-oxo-1,3,8-triazaspiro[4.5]decane-8-carboxylate;
2-(4-cyclopropylphenyl)-3-[2-(4-methoxyphenyl)ethyl]-1-methyl-4-oxo-1,3,8-triazaspiro[4.5]decane-8-carbohydrazide;
2-[4-(difluoromethoxy)phenyl]-3-[2-(4-methoxyphenyl)ethyl]-1-methyl-8-pentanoyl-1,3,8-triazaspiro[4.5]decan-4-one;
8-(cyclopentylcarbonyl)-2-[4-(difluoromethoxy)phenyl]-3-[2-(4-methoxyphenyl)ethyl]-1-methyl-1,3,8-triazaspiro[4.5]decan-4-one;
8-(cyclopropylcarbonyl)-2-(4-isobutylphenyl)-3-[2-(4-methoxyphenyl)ethyl]-1-methyl-1,3,8-triazaspiro[4.5]decan-4-one;
2-(4-cyclopropylphenyl)-3-[2-(4-methoxyphenyl)ethyl]-1-methyl-4-oxo-1,3,8-triazaspiro[4.5]decane-8-carboximidamide;
tert-butyl 2-(4-isobutylphenyl)-3-[2-(4-methoxyphenyl)ethyl]-1-methyl-4-oxo-1,3,8-triazaspiro[4.5]decane-8-carboxylate;
2-(4-cyclopropylphenyl)-N,1-dimethyl-4-oxo-3-(3-phenylpropyl)-1,3,8-triazaspiro[4.5]decane-8-carboxamide;
2-(4-cyclopropylphenyl)-3-[2-(4-methoxyphenyl)ethyl]-N′,1-dimethyl-4-oxo-1,3,8-triazaspiro[4.5]decane-8-carboximidamide;
2-{2-(4-cyclopropylphenyl)-3-[2-(4-methoxyphenyl)ethyl]-1-methyl-4-oxo-1,3,8-triazaspiro[4.5]dec-8-yl}-N,N-dimethylacetamide;
2-(4-cyclopropylphenyl)-3-[2-(4-methoxyphenyl)ethyl]-1-methyl-8-D-prolyl-1,3,8-triazaspiro[4.5]decan-4-one;
2-(4-cyclopropylphenyl)-8-(1H-imidazol-1-ylcarbonyl)-3-[2-(4-methoxyphenyl)ethyl]-1-methyl-1,3,8-triazaspiro[4.5]decan-4-one; or
2-(4-cyclopropylphenyl)-3-[2-(4-methoxyphenyl)ethyl]-8-(methylsulfonyl)-1-(trifluoroacetyl)-1,3,8-triazaspiro[4.5]decan-4-one;
2-(4-methoxyphenyl)-3-[2-(4-methoxyphenyl)ethyl]-1-methyl-1,3,8-triaza-spiro[4.5]decan-4-one;
phenyl N-cyano-2-(4-cyclopropylphenyl)-3-[2-(4-methoxyphenyl)ethyl]-1-methyl-4-oxo-1,3,8-triazaspiro[4.5]-decane-8-carboximidoate;
tert-butyl 2-(4-tert-butylbenzyl)-3-[2-(4-methoxyphenyl)ethyl]-1-methyl-4-oxo-1,3,8-triazaspiro[4.5]decane-8-carboxylate;
(S)-2-(4-cyclopropylphenyl)-3-[2-(4-methoxyphenyl)ethyl]-1-methyl-4-oxo-1,3,8-triaza-spiro[4.5]decane-8-carboxylic acid amide;
(R)-2-(4-cyclopropylphenyl)-3-[2-(4-methoxyphenyl)ethyl]-1-methyl-4-oxo-1,3,8-triaza-spiro[4.5]decane-8-carboxylic acid amide;
2-(4-cyclopropylphenyl)-N-ethoxy-3-(4-methoxyphenethyl)-1-methyl-4-oxo-1,3,8-triazaspiro[4.5]decane-8-carboxamide; or a pharmaceutically acceptable salt form thereof.

65. A pharmaceutical composition comprising one or more compounds according to claim 1 and one or more excipients.

66. A method for treating or preventing atrial antiarrhythmia comprising administering an effective amount of a compound according to formula (I): 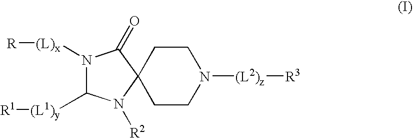
wherein R is optionally substituted phenyl;
R¹ is optionally substituted phenyl;
R² is hydrogen, optionally substituted C₁-C₆ linear or branched alkyl, optionally substituted C₃-C₆ cycloalkyl or —C(O)R²³, wherein R²³ is optionally substituted C₁-C₆ linear or branched alkyl or optionally substituted C₃-C₆ cycloalkyl;
R³ is selected from:
i) hydrogen;
ii) optionally substituted C₁-C₆ linear or branched alkyl, or optionally substituted C₃-C₆ cycloalkyl;
iii) —C(O)R⁴;
wherein R⁴ is optionally substituted C₁-C₆ linear or branched alkyl, optionally substituted C₃-C₆ cycloalkyl, optionally substituted heterocycle, optionally substituted aryl, or optionally substituted heteroaryl;
iv) —C(O)NR⁵R⁶;
wherein R⁵ and R⁶ are each independently selected from:
a) hydrogen;
b) optionally substituted C₁-C₆ linear or branched alkyl;
c) optionally substituted C₃-C₇ cycloalkyl;
d) —OR⁷;
wherein R⁷ is hydrogen or optionally substituted C₁-C₆ linear or branched alkyl;
e) —NR⁸R⁹;
wherein R⁸ and R⁹ are each independently hydrogen, optionally substituted C₁-C₆ linear or branched alkyl, optionally substituted C₁-C₆ linear or branched alkoxy, —OH, or —CO₂R¹⁰, wherein R¹⁰ is optionally substituted C₁-C₆ linear or branched alkyl; or R⁸ and R⁹ can be taken together with the atom to which they are bound to form an optionally substituted ring having from 3 to 7 ring atoms and optionally containing one or more additional heteroatom ring atoms independently selected from N, O, or S; or
f) R⁵ and R⁶ can be taken together with the atom to which they are bound to form an optionally substituted ring having from 3 to 7 ring atoms and optionally containing one or more additional heteroatom ring atoms independently selected from N, O, or S;
v) —C(NR¹¹)R¹²;
wherein R¹¹ is
a) hydrogen;
b) optionally substituted C₁-C₆ linear or branched alkyl, or optionally substituted C₃-C₆ cycloalkyl;
c) —OH; or
d) —CN; and
R¹² is
a) optionally substituted C₁-C₆ linear or branched alkyl, or optionally substituted C₃-C₆ cycloalkyl;
b) —OR¹³;
wherein R¹³ is hydrogen, optionally substituted C₁-C₆ linear or branched alkyl, optionally substituted C₃-C₆ cycloalkyl, or optionally substituted aryl; or
c) —NR¹⁴R¹⁵;
wherein R¹⁴ and R¹⁵ are each independently hydrogen, optionally substituted aryl, optionally substituted C₁-C₆ linear or branched alkyl, or optionally substituted C₃-C₈ cycloalkyl;
vi) —SO₂R¹⁶;
wherein R¹⁶ is optionally substituted aryl, optionally substituted C₁-C₆ linear or branched alkyl, or optionally substituted C₃-C₆ cycloalkyl;
vii) —C(O)R¹⁷;
wherein R¹⁷ is optionally substituted aryl or optionally substituted heteroaryl; or
viii) —C(O)OR¹⁸;
wherein R¹⁸ is optionally substituted C₁-C₆ linear or branched alkyl, optionally substituted C₃-C₆ cycloalkyl, or optionally substituted aryl;
L, L¹, and L² are linking units each independently a unit having the formula:
—[C(R¹⁹)₂]ₙ—
wherein each R¹⁹ is, at each occurrence, independently chosen from hydrogen, methyl, or ethyl;
n is 1 to 4; and
x, y, and z are each independently 0 or 1;
or a pharmaceutically acceptable salt form thereof;
to a subject.

67. A method for preventing or treating thromboembolism, stroke, or cardiac failure comprising administering an effective amount of a compound according to formula (I): 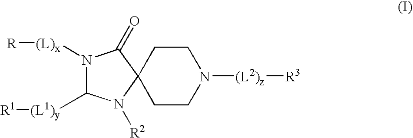
wherein R is optionally substituted phenyl;
R¹ is optionally substituted phenyl;
R² is hydrogen, optionally substituted C₁-C₆ linear or branched alkyl, optionally substituted C₃-C₆ cycloalkyl or —C(O)R²³, wherein R²³ is optionally substituted C₁-C₆ linear or branched alkyl or optionally substituted C₃-C₆ cycloalkyl;
R³ is selected from:
i) hydrogen;
ii) optionally substituted C₁-C₆ linear or branched alkyl, or optionally substituted C₃-C₆ cycloalkyl;
iii) —C(O)R⁴;
wherein R⁴ is optionally substituted C₁-C₆ linear or branched alkyl, optionally substituted C₃-C₆ cycloalkyl, optionally substituted heterocycle, optionally substituted aryl, or optionally substituted heteroaryl;
iv) —C(O)NR⁵R⁶;
wherein R⁵ and R⁶ are each independently selected from:
a) hydrogen;
b) optionally substituted C₁-C₆ linear or branched alkyl;
c) optionally substituted C₃-C₇ cycloalkyl;
d) —OR⁷;
wherein R⁷ is hydrogen or optionally substituted C₁-C₆ linear or branched alkyl;
e) —NR⁸R⁹;
wherein R⁸ and R⁹ are each independently hydrogen, optionally substituted C₁-C₆ linear or branched alkyl, optionally substituted C₁-C₆ linear or branched alkoxy, —OH, or —CO₂R¹⁰, wherein R¹⁰ is optionally substituted C₁-C₆ linear or branched alkyl; or R⁸ and R⁹ can be taken together with the atom to which they are bound to form an optionally substituted ring having from 3 to 7 ring atoms and optionally containing one or more additional heteroatom ring atoms independently selected from N, O, or S; or
f) R⁵ and R⁶ can be taken together with the atom to which they are bound to form an optionally substituted ring having from 3 to 7 ring atoms and optionally containing one or more additional heteroatom ring atoms independently selected from N, O, or S;
v) —C(NR¹¹)R¹²;
wherein R¹¹ is
a) hydrogen;
b) optionally substituted C₁-C₆ linear or branched alkyl, or optionally substituted C₃-C₆ cycloalkyl;
c) —OH; or
d) —CN; and
R¹² is
a) optionally substituted C₁-C₆ linear or branched alkyl, or optionally substituted C₃-C₆ cycloalkyl;
b) —OR¹³;
wherein R¹³ is hydrogen, optionally substituted C₁-C₆ linear or branched alkyl, optionally substituted C₃-C₆ cycloalkyl, or optionally substituted aryl; or
c)—NR¹⁴R¹⁵;
wherein R¹⁴ and R¹⁵ are each independently hydrogen, optionally substituted aryl, optionally substituted C₁-C₆ linear or branched alkyl, or optionally substituted C₃-C₈ cycloalkyl;
vi) —SO₂R¹⁶;
wherein R¹⁶ is optionally substituted aryl, optionally substituted C₁-C₆ linear or branched alkyl, or optionally substituted C₃-C₆ cycloalkyl;
vii) —C(O)R¹⁷;
wherein R¹⁷ is optionally substituted aryl or optionally substituted heteroaryl; or
viii) —C(O)OR¹⁸;
wherein R¹⁸ is optionally substituted C₁-C₆ linear or branched alkyl, optionally substituted C₃-C₆ cycloalkyl, or optionally substituted aryl;
L, L¹, and L² are linking units each independently a unit having the formula:
—[C(R¹⁹)₂]ₙ—
wherein each R¹⁹ is, at each occurrence, independently chosen from hydrogen, methyl, or ethyl;
n is 1 to 4; and
x, y, and z are each independently 0 or 1;
or a pharmaceutically acceptable salt form thereof;
to a subject.

68. A compound having formula (I): 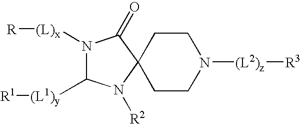
(I)
wherein R is optionally substituted phenyl;
R¹ is optionally substituted phenyl;
R² is hydrogen;
R³ is selected from:
i) hydrogen;
ii) optionally substituted C₁-C₆ linear or branched alkyl, or optionally substituted C₃-C₆ cycloalkyl;
iii) —C(O)R⁴;
wherein R⁴ is optionally substituted C₁-C₆ linear or branched alkyl, optionally substituted C₃-C₆ cycloalkyl, optionally substituted heterocycle, optionally substituted aryl, or optionally substituted heteroaryl;
iv) —C(O)NR⁵R⁶;
wherein R⁵ and R⁶ are each independently selected from:
a) hydrogen;
b) optionally substituted C₁-C₆ linear or branched alkyl;
c) optionally substituted C₃-C₇ cycloalkyl;
d) —OR⁷;
wherein R⁷ is hydrogen or optionally substituted C₁-C₆ linear or branched alkyl;
e) —NR⁸R⁹;
wherein R⁸ and R⁹ are each independently hydrogen, optionally substituted C₁-C₆ linear or branched alkyl, optionally substituted C₁-C₆ linear or branched alkoxy, —OH, or —CO₂R¹⁰, wherein R¹⁰ is optionally substituted C₁-C₆ linear or branched alkyl; or R⁸ and R⁹ can be taken together with the atom to which they are bound to form an optionally substituted ring having from 3 to 7 ring atoms and optionally containing one or more additional heteroatom ring atoms independently selected from N, O, or S; or
f) R⁵ and R⁶ can be taken together with the atom to which they are bound to form an optionally substituted ring having from 3 to 7 ring atoms and optionally containing one or more additional heteroatom ring atoms independently selected from N, O, or S;
v) —C(NR¹¹)R¹²;
wherein R¹¹ is
a) hydrogen;
b) optionally substituted C₁-C₆ linear or branched alkyl, or optionally substituted C₃-C₆ cycloalkyl;
c) —OH; or
d) —CN; and
R¹² is
a) optionally substituted C₁-C₆ linear or branched alkyl, or optionally substituted C₃-C₆ cycloalkyl;
b) —OR¹³;
wherein R¹³ is hydrogen, optionally substituted C₁-C₆ linear or branched alkyl, optionally substituted C₃-C₆ cycloalkyl, or optionally substituted aryl; or
c)—NR¹⁴R¹⁵;
wherein R¹⁴ and R¹⁵ are each independently hydrogen, optionally substituted aryl, optionally substituted C₁-C₆ linear or branched alkyl, or optionally substituted C₃-C₈ cycloalkyl;
vi) —SO₂R¹⁶;
wherein R¹⁶ is optionally substituted aryl, optionally substituted C₁-C₆ linear or branched alkyl, or optionally substituted C₃-C₆ cycloalkyl;
vii) —C(O)R¹⁷;
wherein R¹⁷ is optionally substituted aryl or optionally substituted heteroaryl; or
viii) —C(O)OR¹⁸;
wherein R¹⁸ is optionally substituted C₁-C₆ linear or branched alkyl, optionally substituted C₃-C₆ cycloalkyl, or optionally substituted aryl;
L, L¹, and L² are linking units each independently a unit having the formula:
—[C(R¹⁹)₂]ₙ—
wherein each R¹⁹ is, at each occurrence, independently chosen from hydrogen, methyl, or ethyl;
n is 1 to 4; and
x, y, and z are each independently 0 or 1; or a pharmaceutically acceptable salt form thereof.

69. The compound of claim 68 wherein R³ is hydrogen, —C(O)CH₃, —C(O)cyclopropyl, —C(O)NH₂, —C(O)NHCH₃, —C(O)N(CH₃)₂, —C(O)NH[CH(CH₃)₂], —C(O)NHCH₂CH₃, —C(O)N(CH₂CH₃), —C(O)OCH₃, —C(O)OCH₂CH₃, —C(O)OCH(CH₃)₂, —C(O)OC(CH₃)₃, —C(O)NHOH, —C(O)NHOCH₃, —C(O)N(CH₃)OCH₃, —C(O)NHNH₂, —C(O)NHOCH₂CH₃, —C(O)NCH₃OCH₃, —C(O)NHNHC(O)OCH₃, —C(O)NHNHC(O)OC(CH₃)₃, —C(NCN)NH₂, —C(NCN)NHCH₃, —C(NCN)NHC₆H₅, —C(O)aziridin-1-yl, —C(O)azetidin-1-yl, —C(O)pyrrolidin-1-yl, —C(O)piperidin-1-yl, —C(O)piperazin-1-yl, —C(O)morpholin-4-yl, —C(O)imidazolin-1-yl, —C(O)isoxazolin-5-yl, —SO₂CH₃, —SO₂CH₂CH₃, —SO₂CH(CH₃)₂, or SO₂C₆H₅.

70. The compound of claim 68 that is
2-(4-Diethylaminophenyl)-3-[2-(4-methoxyphenyl)ethyl]-4-oxo-1,3,8-triaza-spiro[4.5]decane-8-carboxylic acid tert-butyl ester;
2-(4-tert-Butylphenyl)-3-[2-(4-methoxyphenyl)ethyl]-4-oxo-1,3,8-triaza-spiro[4.5]decane-8-carboxylic acid tert-butyl ester;
2-(4-Difluoromethoxyphenyl)-3-[2-(4-methoxyphenyl)ethyl]-4-oxo-1,3,8-triaza-spiro[4.5]decane-8-carboxylic acid tert-butyl ester;
tert-butyl 2-(4-methoxyphenyl)-3-[2-(4-methoxyphenyl)ethyl]-4-oxo-1,3,8-triazaspiro[4.5]decane-8-carboxylate;
2-(4-cyclopropylphenyl)-3-[2-(4-methoxyphenyl)ethyl]-4-oxo-1,3,8-triazaspiro[4.5]decane-8-carboxamide;
methyl 2-(4-cyclopropylphenyl)-3-[2-(4-methoxyphenyl)ethyl]-4-oxo-1,3,8-triazaspiro[4.5]decane-8-carboxylate;
2-(4-tert-butylphenyl)-3-[2-(4-methoxyphenyl)ethyl]-1,3,8-triazaspiro[4.5]decan-4-one;
2-(4-tert-butylphenyl)-8-(ethylsulfonyl)-3-[2-(4-methoxyphenyl)ethyl]-1,3,8-triazaspiro[4.5]decan-4-one;
tert-butyl 2-(4-cyclopropylphenyl)-3-[2-(4-methoxyphenyl)ethyl]-4-oxo-1,3,8-triazaspiro[4.5]decane-8-carboxylate;
2-(4-cyclopropylphenyl)-3-[2-(4-methoxyphenyl)ethyl]-8-(methylsulfonyl)-1,3,8-triazaspiro[4.5]decan-4-one;
tert-butyl 2-(4-methoxyphenyl)-4-oxo-3-(3-phenylpropyl)-1,3,8-triazaspiro[4.5]decane-8-carboxylate;
2-(4-tert-butylphenyl)-8-(cyclopropylcarbonyl)-3-[2-(4-methoxyphenyl)ethyl]-1,3,8-triazaspiro[4.5]decan-4-one; or
2-(4-cyclopropylphenyl)-3-[2-(4-methoxyphenyl)ethyl]-1,3,8-triazaspiro[4.5]decan-4-one;
tert-butyl 2-(4-tert-butylphenyl)-4-oxo-3-(3-phenylpropyl)-1,3,8-triazaspiro[4.5]decane-8-carboxylate;
2-(4-tert-Butylbenzyl)-3-[2-(4-methyoxyphenyl)ethyl]-4-oxo-1,3,8-triaza-spiro[4.5]decane-8-carboxylic acid tert-butyl ester;
2-(4-tert-Butylphenyl)-8-methanesulfonyl-3-[2-(4-methoxyphenyl)ethyl]-1,3,8-triaza-spiro[4.5]decan-4-one;
or a pharmaceutically acceptable salt form thereof.
